(19) United States
(12) Patent Application Publication
(10) Pub. No.: US 2016/0220489 A1
(43) Pub. Date: Aug. 4, 2016

(54) Intranasal Formulation for the Treatment of Cardiopulmonary Resuscitation (CPR), Cardiac Life Support (CLS), Anaphylaxis and/or Anaphylactoid Reactions

(71) Applicants: G2B Pharma Inc., Corte Madera, CA (US); SHIN NIPPON BIOMEDICAL LABORATORIES LTD. (SNBL), Tokyo (JP)

(72) Inventors: Nigel T. Fleming, Girona (ES); Shunji Haruta, Kagoshima-ken (JP)

(73) Assignee: G2B Pharma Inc., Corte Madera, CA (US)

(21) Appl. No.: 14/916,098

(22) PCT Filed: Sep. 2, 2014
(86) PCT No.: PCT/US2014/053700

§ 371 Date: Mar. 2, 2016

Related U.S. Application Data
(60) Provisional application No. 61/873,167, filed on Sep. 3, 2013.
(60) Provisional application No. 62/044,382, filed on Sep. 1, 2014.

(51) Int. Cl. A61K 9/14; A61K 31/137; A61K 38/08; A61N 1/39; A61K 31/417; A61K 31/277; A61M 15/08; A61M 15/00; A61K 9/00; A61K 31/439

(52) U.S. Cl. CPC A61K 9/14; A61K 9/0043; A61K 31/137; A61K 38/08; A61K 31/439; A61K 31/417; A61K 31/277; A61M 15/08; A61M 15/0045; A61M 15/0068; A61N 1/3975; A61M 2202/064

(57) ABSTRACT

Disclosed herein are dry powder compositions and unit doses that comprise vasoactive agents, and/or anti-anaphylactic and/or anti-anaphylactoid agents suitable for intranasal administration, methods of making the compositions, and methods of using the compositions to treat disorders, for example anaphylaxis, anaphylactoid reactions, bronchospasm, cardiac arrest, hypotensive shock, or other situations requiring the need to implement cardiopulmonary resuscitation (CPR) and/or basic or advanced cardiac life support (ACLS).

### CROSS REFERENCE TO RELATED APPLICATIONS

This application claims the benefit of PCT Patent Application No. PCT/US14/53700 filed on Sep. 2, 2014, U.S. Provisional Patent Application No. 62/044,382 on Sep. 1, 2014, and U.S. Provisional Patent Application No. 61/873,167 filed on Sep. 3, 2013.

### FIELD OF THE INVENTION

Disclosed herein are methods, kits, dry powder compositions and unit doses that comprise vasoactive agent, and/or anti-anaphylactic and/or anti-anaphylactoid agents suitable for intranasal administration, methods of making the compositions, and methods of using the compositions to treat disorders, for example anaphylaxis, anaphylactoid reactions, bronchospasm, cardiac arrest, hypotensive shock, or other situations requiring the need to implement cardiopulmonary resuscitation (CPR) and/or basic or advanced cardiac life support (ACLS).

### BACKGROUND OF THE INVENTION

Anaphylaxis is a severe, rapid onset allergic reaction to insect stings or bites, foods, drugs, allergens, and can be idiopathic or exercise-induced. About 3 million American children suffer from food allergies (from peanuts, tree nuts, milk, eggs, fish, seafood and gluten) and according to a study released in 2008 by Centers for Disease Control and Prevention, there has been about an 18% increase in food allergy since 1997. Anaphylaxis occurs in about 1-16% of the US population and carries a 1% mortality rate. An anaphylactoid reaction is a reaction resembling generalized anaphylaxis but not caused by IgE-mediated allergic reaction but rather by a nonimmunologic mechanism.

Further, out-of-hospital cardiac arrest (OHCA) is an important public health concern, and according to a study released in 2011 by Center for Disease Control and Prevention, OHCA occurs in more than 300,000 individuals in the United States every year (McNally B. et al. 2011, MMWR Surveill Summ. 60(8): 1-19).

During an OHCA event there is minimal to no cardiac mechanical activity, which corresponds to a critical reduction in circulation (Roger V. L. et al. 2011, Circulation 123: e18-e209; Jacobs I. et al. 2004, Circulation 110(21): 3385-97). Data suggests that up to 92% of OHCA patients die, especially when the “Chain of Survival” (see FIG. 1) is not effectively implemented (Roger V L et al. 2011, Circulation 123: e18-e209). However, significant improvement can be achieved and survival rate can be as high as 50% when the components of the Chain of Survival are implemented in an effective and timely manner in a witnessed cardiac arrest secondary to ventricular fibrillation (VF) (AHA Guidelines for CPR and Emergency Cardiovascular care. Circulation 2005, 112(24 Suppl): IV1-203; Wik L. et al. 2005, JAMA 293: 299-304; Abella B. S. et al. 2005, JAMA 293: 305-310; Callaway C. W. et al. 2010, Resuscitation 81: 524-529). The potential challenges related to implementing the chain of survival include: initiating CPR, providing effective compressions to improve circulation (CPP), limiting compression interruptions, and rapid delivery of ACLS.

As shown in FIG. 1, one of the essential links in the Chain of Survival is the rapid delivery of ACLS including effective CPR and early defibrillation using automated external defibrillator (AED). A generic AED protocol followed during a typical OHCA event is shown in Table 1. A key component of the ACLS protocol, also addressed in this application, is the timely and effective delivery of epinephrine, a primary drug advocated for patients during resuscitation due to its alpha-adrenergic receptor stimulating properties (Yakaitis R. W. et al. 1979, Crit. Care Med. 7: 293-96; Otto C. W. et al. 1984, Ann. Emerg. Med. 9 pt. 2: 840-843; Field J. M. et al. 2010, Circulation 122(18 Supple 3): S640-56).

TABLE 1
Generic AED Protocol

(For Adults and kids over age eight (8) or greater, or fifty-five (55 lbs.) twenty-five (25 kg.)
or greater of body weight)
Step 1: Confirm patient is pulseless and apneic
Step 2: Turn on AED (If properly functioning the device prompts “(i) Apply Pads and (ii)
Plug in connector”)
Step 3: Attach pads (upper right and lower left chest; insert the defibrillator pads connector
into the socket located)
Step 4: AED prompts: “Analyzing rhythm; do not move or touch patient”.
Step 5: If shockable rhythm is advised, AED prompts, “Do not touch victim; press shock
button now” (after this prompt, make sure everyone is clear of patient and press the shock button
to deliver the shock)
Step 6: The cardiac rhythm will again be assessed automatically. The above actions will
repeat for a total of 3 times as long as a shockable rhythm is present.
Step 7: After a third shock AED prompts, “Paused, it is safe to touch patient, check airway,
breathing, pulse, if needed begin CPR”
At this point if the patients have not been resuscitated manual CPR is started until
paramedics arrive on the scene. It is envisioned that with the current described invention, nasal
epinephrine would be administered as this time as prompted by the AED. This will allow for an
important link in the Chain of Survival to be administered in a timely manner.
AED Application in Pediatric Patients (age eight (8) or less (including newborns) or
fifty-five (55 lbs) twenty-five (25 kg.) or less body weight)
When following AED protocol for pediatric cardiac arrest patients, use of (AED TYPE)
Child/Infant Electrode Pads that are designed to automatically reduce AED defibrillation energy
to a more clinically appropriate output is recommended.
The best pediatric pad placement is achieved utilizing an anterior/posterior placement (one
pad on the chest and the other on the back) for infants' and children with tiny torsos. This method
of placement prevents pad overlap, which could lead to arching. However, it is still acceptable
to use conventional pad placement (anterior/anterior) when needed.

Epinephrine (adrenaline) is the uncontested cornerstone for the treatment of anaphylaxis and/or anaphylactoid reactions, and can be lifesaving (Joint Task Force on Practice Parameters, 2005, J Allergy Clin Immunol 115: S483-S523; Lieberman P. 2003, Curr Opin Allergy Clin Immunol 3: 313-318; Simons F. E. R. 2004, J Allergy Clin Immunol 113: 837-844). Any delay in administration of epinephrine may be fatal. In 2003, 1.4 million intramuscular (IM) doses (EpiPen™) were prescribed in the United States and it increased to 1.9 million by 2007. About 100-200 people die annually in the US from food allergies.

Epinephrine has also been clearly shown in animal models to increase coronary perfusion pressure (CPP) (Otto C. W. et al. 1984, Ann. Emerg. Med. 9 pt. 2: 840-843; Paradis N. A. et al. 1991, JAMA 265(9): 1139-44), which is significantly associated with return of spontaneous circulation (ROSC) (Paradis N. A. et al. 1990, JAMA 263(8): 1106-13; Niemann J. T. et al. 2002, Resuscitation 53(2): 153-7; Callaway C. W. 2013, Curr. Opin. Cardiol. 28(1): 36-42). It has been shown in the clinical setting that earlier implementation of the Chain of Survival and a consequent increase of CPP and circulation greatly increases the chance for survival in patients. The need to efficiently and effectively deliver epinephrine during cardiac arrest is more apparent especially since brain injury can begin as early as 4-6 minutes following a cardiac arrest event. The American Heart Association (AHA) algorithms has recommended the administration of intravenous (IV) epinephrine as a treatment option for cardiac arrest secondary to ventricular fibrillation, pulseless electrical activity, and asystole.

However, intravenous (IV) delivery also has significant drawbacks in the emergency care setting. The most critical drawbacks that contribute to delays in intravenous drug delivery include: (i) difficulty in obtaining an IV access site even for an experienced caregiver (Hoskins, Resuscitation 2012) due to the lack of peripheral circulation (e.g. cardiovascular collapse) especially in obese patients or those with poor venous access and (ii) need for obtaining an IV access site when the emergency personals are initially engaged in a number of other activities such as chest compressions, defibrillation, and ventilation. Further, a study (Lapostolle F. et al. 2007, Lancet 369(9571): 1430) found that the median time to establish an IV line was 2 minutes for initial attempts and 5 minutes when additional attempts were necessary. In a prospective observational study using national registry data based in Japan showed that in over 400,000 cases of OHCA 81% of patients did not have an intravenous line inserted (Hagihara A. et al. 2012, JAMA 307(11): 1161-1168). According to another recent study of OHCA (Nakahara S, et al. 2012 Acad. Emerg. Med. 19(7):782-92), the mean time to first dose of intravenous epinephrine was 21 minutes (range 16-27 minutes) with an EMS mean response time of 8 minutes (range 6-10 minutes). In addition, when prefilled syringes are not available there are other inherent time delays to administering IV drugs including the preparation of drugs for IV delivery. The steps required to prepare and deliver IV epinephrine are illustrated in Table 2. As shown, the large number of steps involved takes time, creates the potential for error and increases the risk for needle sticks.

TABLE 2
Steps Required for Injection of Epinephrine
To deliver epinephrine in this setting the following steps are necessary.
These steps introduce potential challenges or complications and/or delay the
successful and rapid delivery of the drug.
Steps Required in Use of Epinephrine from Ampoules:
Tap ampoule to dislodge trapped solution
Place gauze around neck
Snap of neck of ampoule
Prepare syringe and insert needle into ampoule
Withdraw drug
Steps Required in Use of Epinephrine from Vials:
Confirm medication identification
Prepare the syringe and hypodermic needle
Clean vial's rubber top
Insert needle into rubber top and inject air from the syringe into the vial
Withdraw the correct volume of medication

Other routes of epinephrine administration during a cardiac arrest including endotracheal and intraosseous delivery also have significant drawbacks leading to significant time delays. For instance, endotracheal administration requires patients to be intubated, which is technically difficult in the field and may not be done. In addition, the pharmacokinetics are highly variable through this route of administration and it is recommended as a last line for drug administration. Intraosseous administration is also technically difficult, as it is associated with highly variable pharmacokinetic profile and it is a skill that is not often utilized by emergency personal in the field. Intraosseous administration is preferred over endotracheal and there are several devices available to facilitate drilling into sternum, tibia and humerus bone. However, given the invasive and technical nature of this approach it is still not considered first line. Perhaps, the most important variable with each of these routes of administration is the time delay in drug administration. Since these are not preferred routes of administration, in practically every case in which these routes are considered there is a prior failure to obtain IV access contributing to significant time delay to drug administration. As previously discussed, every minute is critical during a cardiac arrest and it is crucial that every link in the Chain of Survival is optimized including drug delivery (Hess E. P. and White R. D. 2010, J. Cardiovasc. Electrophysiol. 21(5): 590-95). A novel approach addressed in this application is to deliver vasoactive therapy during cardiac arrest in a manner that is non-technical, and can be rapidly administered at any point in the ACLS link, by nasal administration. The nasal route of drug administration does not present with the same drawbacks that are inherent to IV, endotracheal, and intraosseous routes of administration. In addition, nasal administration requires minimal technical skills and can be administered by lay personnel. Nasal administration is a viable option to optimize the timing and delivery of vasoactive drugs in the setting of OHCA.

In addition, the currently marketed EpiPen™ autoinjector comes in two fixed doses of 0.15 mg for pediatric patients and 0.3 mg for adults, which often forces physicians to decide whether to under- or overdose a patient based on weight, especially in children. Hence, there exists a need in the art for convenient and easy delivery of correct dosage forms that does not require prior training in the use of the device.

Previous animal studies have demonstrated that intranasal delivery of epinephrine solution can be an effective route of drug administration during cardiac arrest (Bleske B. E. et al. 1992, Ann. Emerg. Med. 21(9): 1125-1130; and Bleske B. E. et al. 1996, Am. J. Emerg. Med. 14: 133-138; and Bleske B. E. et al. 1996, Pharmacother. 16(6): 1039-1045). Although these authors observed a dose response, the absolute bioavailability appeared to be quite low, despite the use of 1% taurodeoxycholic acid solution (bile salts), which is now known to be a damaging mucosal permeation enhancer. Moreover, to minimize the severe local vasoconstriction caused by epinephrine that could potentially limit the mucosal absorption of epinephrine, they used pretreatment with intranasal phentolamine. The phentolamine pretreatment was administered 1 min prior to intranasal epinephrine dosing to enhance epinephrine absorption. To prevent its local degradation on the external nasal mucosa, the investigators used large loading doses of phentolamine ranging from 0.25 to 2.5 mg/kg/nostril, which amount to 15 mg for a dog weighing 21 kg. The loading doses of epinephrine studied were 0.075, 0.75 and 7.5 mg/kg/nostril, which amount to 157 mgs/nostril for a dog weighing 21 kg. The greatest cardiac effects and the greatest epinephrine plasma concentrations of about ˜1,400 ng/mL were observed at 0.25 mg/kg/nostril of phentolamine and 7.5 mg/kg/nostril of epinephrine. For desired treatment the authors used 7.5 mg/kg/nostril of epinephrine with 1% taurodeoxycholic acid as permeation enhancer after pretreatment with 0.75 mg/kg/nostril in about 1 ml each application.

Although this study revealed the systemic delivery of aqueous epinephrine by the nasal route of administration, it had significant limitations for translation into clinical practice, including: (i) the dosage of epinephrine was not optimized (it is also unclear whether phentolamine was used at its lowest level, because a systemic exposure of phentolamine would have competed with epinephrine actions); (ii) the staged pre-dosing of phentolamine followed by epinephrine is impractical for real-world emergency treatment; (iii) the use of large loading doses of epinephrine (157 mgs/nostril for a dog weighing 21 kgs) and vasodilator (15 mg/nostril for a dog weighing 21 kgs); (iv) the use of large volumes (e.g. 1.0 mL of solution per nostril) caused the epinephrine to crystallize out; (v) the large volume in each nostril is also impractical because modern nasal aqueous sprays use only 100-250 μl volume. And even at this lower volume quite a significant percentage of the aqueous dose slides off the more dense nasal mucosa and is swallowed); and (vi) the use of bile salts as mucosal permeation enhancers caused severe nasal mucosal tissue damage, and raises the question to what extent was the systemic delivery achieved due to the destruction of this tissue barrier, rather than by the actual penetration by epinephrine. This safety aspect of the study was a fatal flaw for clinical translation of this technology. This technology is dormant with no follow-up studies since 1996.

An epinephrine formulation that is delivered to the lungs of a subject and administered by breath-activated devices is described in two U.S. Pat. Nos. 7,954,491 and 7,947,742. However, such pulmonary delivery of epinephrine is not feasible in persons already in cardiac arrest. Also, as inferred by the Bleske et al. 1996 study, any epinephrine formulation that does not contain other epinephrine-enabling agents such as a vasodilator, will not be effective to counter the cardiac shock within the typical 1-5 minutes needed for rescue of cardiac arrest. In a recent study by Nakponetong K. et al. 2010 J Allergy Clin Immunol 125(2): Abstract 859, a nasal spray with a high loading dose of epinephrine (5 mg) was given to normal human subjects and was compared with intramuscular epinephrine. The study revealed a peak plasma concentration (Tₘₐₓ) reached in 70±17 minutes. A Tₘₐₓ of 70±17 minutes, even at the higher loading dose of epinephrine, is insufficient to be of any utility in cardiac arrest or anaphylactic shock. Paradoxically, the data on the PK of the IM epinephrine injection was also unacceptable, and the methodology of the study is called into question.

Hence, despite the theoretical promise for the intranasal route for delivering epinephrine, there is a need for improving the intranasal (IN) epinephrine formulation for the treatment of anaphylaxis and/or anti-anaphylactoid reactions. As noted above, the investigation by Bleske B. E. et al. 1996, also had a number of significant practical limitations that prevented their clinical translation.

The formulations and/or dosage units herein, however, have solved critical limitations of the above-mentioned references. The dry powder formulations herein permit lowering the loading dose of the vasoactive agent, and/or the anti-anaphylactic and/or anti-anaphylactoid agents (e.g., epinephrine) by the optional addition of a topically-acting vasodilator (e.g., phentolamine) with epinephrine in the same dose. In addition to vasoactive agent, and/or anti-anaphylactic and/or anti-anaphylactoid agents (e.g., epinephrine) and vasodilator, the present formulation can contain one or more enabling agents including, mucoadhesives, mucosal transit slowing agents, modern permeation enhancers, and/or other pharmaceutically acceptable excipients that are non-toxic to nasal tissues. Addition of suitable enabling agents to the dry powder formulations and/or dosage units can surprisingly aid in muco-adhesion, dissolution and absorption.

The development of intranasal epinephrine, disclosed in this application, will provide important therapeutic advantages for patients requiring advanced cardiac life support (ACLS). Specifically, intranasal epinephrine may provide to be therapeutically advantageous in the following settings: (i) Secondary route of administration in ACLS setting, especially in those patients in whom intravenous access is delayed or not obtainable. (ii) First-line therapy ACLS setting: intranasal epinephrine can provide time optimization of drug delivery to the systemic circulation in an ACLS setting. As soon as the need for epinephrine is identified, nasal administration can occur in less than one minute. This is much sooner than the time it would take to obtain IV access and deliver the drug by the IV route. Nasal epinephrine can be initially given thus saving time followed up by IV administration once an IV route is established. (iii) Treatment option for AED system: since intranasal epinephrine is a non-technical route of administration, a layperson may administer epinephrine during an emergency setting. Specifically, intranasal epinephrine may be added as part of the AED algorithm. In those situations where the AED fails to terminate the ventricular arrhythmia, nasal epinephrine can be administered to help maintain coronary perfusion pressure along with CPR until trained medical help arrives.

Placement of intranasal epinephrine product with an AED can offer additional treatment options for patients in cardiac arrest. For instance, in an OHCA situation, failure of the AED to cardiovert patients to a non-lethal rhythm would result in the lay person being directed to administer intranasal epinephrine and then provide additional defibrillations per AED algorithm. In addition, when paramedics arrive on the scene, epinephrine would already have been administered. In this situation, two action items in the Chain of Survival, i.e., defibrillation and drug administration, has been optimized.

Another consideration for the use of intranasal epinephrine may be in the setting of severe hypotension (hypotensive shock) secondary to a number of causes including, trauma, hypovolemia, bradycardia, or septic shock where intravenous access is not readily available in a timely fashion. One setting where intranasal epinephrine may be valuable is during active combat. Administering nasal epinephrine in the battlefield to help maintain blood pressure may be practical given the ease of administration by the nasal route. As previously discussed, epinephrine is an ideal candidate to help raise systolic pressures given that epinephrine is a potent vasoconstrictor through the activation of mainly alpha-1 receptors. In addition, previous studies as cited above have shown the proof of concept that nasal epinephrine can be effectively given by nasal route and achieve therapeutic levels to raise pressure even during cardiac arrests. Finally, epinephrine beta adrenergic effects may be beneficial in counteracting bronchoconstriction effects following certain chemical exposures.

Herein, the resulting nasal route of administration to achieve therapeutic doses can be: (i) rapid; (ii) painless (no needle-phobia); (iii) administered by non-professionals; (iv) can use practical doses of an intranasal formulation comprising a vasoactive agent, and/or an anti-anaphylactic and/or anti-anaphylactoid agent (e.g., epinephrine).

### SUMMARY OF THE INVENTION

The inventive embodiments provided in this Summary of the Invention are meant to be illustrative only and to provide an overview of selective embodiments disclosed herein. The Summary of the Invention, being illustrative and selective, does not limit the scope of any claim, does not provide the entire scope of inventive embodiments disclosed or contemplated herein, and should not be construed as limiting or constraining the scope of this disclosure or any claimed inventive embodiment.

In one aspect, provided herein is an intranasal dry powder composition comprising an anti-anaphylactic or anti-anaphylactoid agent. In some embodiments, the anti-anaphylactic or anti-anaphylactoid agent is epinephrine or a pharmaceutically acceptable salt thereof. In some embodiments, the anti-anaphylactic or anti-anaphylactoid agent does not comprise cocaine or a derivative thereof. In some embodiments, a single dose of the anti-anaphylactic or anti-anaphylactoid agent is about 0.01 mg to about 10 mg. In another aspect, the amount of an anti-anaphylactic and/or anti-anaphylactoid agent (e.g., epinephrine) can be at least about: 0.01 mg, 0.05 mg, 1.0 mg, 2.0 mg, 5.0 mg, or 10 mg in the compositions. In some embodiments, the anti-anaphylactic and/or anti-anaphylactoid agent (e.g., epinephrine) present in the compositions can be about: 0.01 mg to 0.05 mg, 0.05 mg to 0.75 mg, 0.75 mg to 1.5 mg, 1.5 mg to 3.0 mg, 3.0 to 4.5 mg, 4.5 to 6.0 mg, 6.0 to 7.5 mg, 7.5 to 9.0 mg, or 9.0 to 10.0 mg. In some embodiments, the amount of an anti-anaphylactic and/or anti-anaphylactoid agent (e.g., epinephrine) can be about: 0.15, 0.3, 0.5, 0.75, 1, 1.5, 2, 3, 4, 5, 6, 7, 8, 9, or 10 mg in the compositions. In one instance, a single dose of the anti-anaphylactic or anti-anaphylactoid agent is about 0.75 mg. In one instance, a single dose of the anti-anaphylactic or anti-anaphylactoid agent is about 1.5 mg. In another instance, a single dose of the anti-anaphylactic or anti-anaphylactoid agent is about 3.0 mg. In another related aspect, the dose of an anti-anaphylactic and/or anti-anaphylactoid agent (e.g., epinephrine) can be adjusted according to the weight of the patient at an increment of at least 0.01 mg/kg, or one wherein the dose can be repeated a number of times if the patient can be refractory or experiences rebound anaphylaxis and/or anaphylactoid reactions. In some embodiments, the composition, in the form of a single dose, contains about 0.01 mg to about 10 mg of the anti-anaphylactic or anti-anaphylactoid agent. In some embodiments, the composition, in the form of a single dose, contains about 0.75 mg, 1.5 mg, or 3.0 mg of the anti-anaphylactic or anti-anaphylactoid agent.

A unit dosage herein can range from about 0.01 mg to about 1 mg, for example about: 0.1, 0.2, 0.3, 0.4, 0.5, 0.6, 0.7, 0.8, 0.9, or 1 mg, of a composition. A unit dosage can also be at least about: 0.01 0.1, 0.5, or 1 mg, of a composition. Administration of the compositions herein can be repeated, e.g., every 5-20 minutes as necessary. In some embodiments, antihistamines (e.g., H₁ and/or H₂ receptor antagonists) and/or corticosteroids can be used in conjunction with an anti-anaphylactic and/or anti-anaphylactoid agent (e.g., epinephrine), a formulation or a unit dose containing thereof, for managing patients suffering from anaphylactic shock and/or anaphylactoid reactions.

In some embodiments, the anti-anaphylactic or anti-anaphylactoid agent is about 0.25% to about 50% w/w of the weight of the composition, for example about: 0.25%, 0.5%, 1%, 2%, 3%, 4%, 5%, 6%, 7%, 7.5%, 8%, 9%, 10%, 11%, 12%, 13%, 14%, 15%, 16%, 17%, 18%, 19%, 20%, 25%, 30%, 35%, 40%, 45%, or 50% w/w, based on the weight of the formulations and/or dosage units. For example, the anti-anaphylactic or anti-anaphylactoid agent can be about 4%, about 7.5%, or about 15% w/w of the weight of the composition. In some embodiments, an anti-anaphylactic and/or anti-anaphylactoid agent (e.g., epinephrine) can be present in an amount of at least about: 0.25% w/w, 1% w/w, 5% w/w, 10% w/w, 20% w/w, 30% w/w, 40% w/w, or 50% w/w based on the weight of the formulations and and/or dosage units. In some embodiments, an anti-anaphylactic and/or anti-anaphylactoid agent (e.g., epinephrine) can be present in an amount of about: 0.25% to 1% w/w, 1% to 5% w/w, 5% to 10% w/w, 10% to 20% w/w, 20% to 30% w/w, 30% to 40% w/w, or 40% to 50% w/w based on the weight of the formulations and/or dosage units.

In some embodiments, the dry powder composition disclosed herein when administered to a patient, may produce a maximal blood concentration (Cₘₐₓ) of the anti-anaphylactic or anti-anaphylactoid agent (e.g., epinephrine) which can be at least about: 2- to 3-fold, 3- to 5-fold, 5- to 7-fold, or 7- to 10-fold more than the baseline level of the anti-anaphylactic or anti-anaphylactoid agent in the patient. In some embodiments, the maximal blood concentration (Cₘₐₓ) of the anti-anaphylactic or anti-anaphylactoid agent (e.g., epinephrine) which may be at least about 2-, 3-, 4-, 5-, 6-, 7-, 8-, 9-, or 10-fold more than the baseline level of the anti-anaphylactic or anti-anaphylactoid agent in the patient. In one embodiment, the dry powder composition when administered to a patient, produces a maximal blood concentration (Cₘₐₓ) of the anti-anaphylactic or anti-anaphylactoid agent at least 2 fold more than the baseline level of the anti-anaphylactic or anti-anaphylactoid agent in the patient. In one embodiment, the compositions herein may increase the blood concentration of an anti-anaphylactic and/or anti-anaphylactoid agent (e.g., epinephrine) by about 0.01 to 0.1 μg/mL. In one embodiment, the compositions herein may increase the blood concentration of an anti-anaphylactic and/or anti-anaphylactoid agent (e.g., epinephrine) by about 0.01, 0.02, 0.03, 0.04, 0.05, 0.06, 0.07, 0.08, 0.09, or 0.1 μg/mL.

In some embodiments, the dry powder composition disclosed herein when administered to a patient, reaches a maximal blood concentration of the anti-anaphylactic or anti-anaphylactoid agent (e.g., epinephrine) in less than about 60 minutes (Tₘₐₓ) after administration. In some embodiments, when administered to a patient, reaches a maximal blood concentration (Tₘₐₓ) of the anti-anaphylactic or anti-anaphylactoid agent in less than about 60, 50, 40, 30, 20, 15, 10, 5, or 3 minutes (Tₘₐₓ) after administration. In one embodiment, the dry powder composition when administered to a patient, reaches a maximal blood concentration (Tₘₐₓ) of the anti-anaphylactic or anti-anaphylactoid agent in less than about 20 minutes after administration. In some embodiments, the dry powder composition when administered to a patient, reaches a mean AUC_{(0-180 minutes)} of the anti-anaphylactic or anti-anaphylactoid agent which is at least 20%, 30%, 40% 50%, 60%, 70%, 80%, 90%, 100%, 110%, 120%, 130%, 140% or 150% of the mean AUC_{(0-180 minutes)} of an IV, IM, or SQ injected anti-anaphylactic or anti-anaphylactoid agent. In some embodiments, the dry powder composition when administered to a patient, reaches a mean AUC_{(0-inf)} of the anti-anaphylactic or anti-anaphylactoid agent which is at least 20%, 30%, 40% 50%, 60%, 70%, 80%, 90%, 100%, 110%, 120%, 130%, 140% or 150% of the mean AUC_{(0-inf)} of an IV, IM, or SQ injected anti-anaphylactic or anti-anaphylactoid agent. In some embodiments, the IV, IM, or SQ injected anti-anaphylactic or anti-anaphylactoid agent contains 0.10 mg, 0.15 mg, 0.20 mg, 0.25 mg, 0.30 mg, 0.35 mg, 0.40 mg, 0.45 mg, 0.50 mg, 0.55 mg, 0.60 mg, 0.65 mg, 0.70 mg, 0.75 mg, 0.80 mg, 0.85 mg, 0.90 mg, 0.95 mg or 1.0 mg of the anti-anaphylactic or anti-anaphylactoid agent. For example, the dry powder composition when administered to a patient, reaches a mean AUC_{(0-180 minutes)} of the anti-anaphylactic or anti-anaphylactoid agent which is at least 80% of the mean AUC_{(0-180 minutes)} of a 0.15 mg IV injected anti-anaphylactic or anti-anaphylactoid agent. In another instance, the dry powder composition when administered to a patient, reaches a mean AUC_{(0-inf)} of the anti-anaphylactic or anti-anaphylactoid agent which is at least 80% of the mean AUC_{(0-inf)} of a 0.15 mg IV injected anti-anaphylactic or anti-anaphylactoid agent. In some embodiments, the IV, IM, or SQ injected anti-anaphylactic or anti-anaphylactoid agent is epinephrine injected by EpiPen™ autoinjector. In some embodiments, the dry powder composition when administered to a patient, reaches a mean AUC_{(0-180 minutes)} of the anti-anaphylactic or anti-anaphylactoid agent which is at least 100,000 pg·min/mL, 200,000 pg·min/mL, 300,000 pg·min/mL, 400,000 pg·min/mL, 500,000 pg·min/mL, 600,000 pg·min/mL, 700,000 pg·min/mL, 800,000 pg·min/mL, 900,000 pg·min/mL, 1,000,000 pg·min/mL. In some embodiments, the dry powder composition when administered to a patient, reaches a mean AUC_{(0-inf)} of the anti-anaphylactic or anti-anaphylactoid agent which is at least 100,000 pg·min/mL, 200,000 pg·min/mL, 300,000 pg·min/mL, 400,000 pg·min/mL, 500,000 pg·min/mL, 600,000 pg·min/mL, 700,000 pg·min/mL, 800,000 pg·min/mL, 900,000 pg·min/mL, 1,000,000 pg·min/mL, 1,200,000 pg·min/mL, 1,400,000 pg·min/mL, 1,600,000 pg·min/mL, 1,800,000 pg·min/mL, 2,000,000 pg·min/mL.

In certain embodiments, the dry powder compositions and/or dosage units herein can raise the blood concentration of an anti-anaphylactic and/or anti-anaphylactoid agent (e.g., epinephrine) to about 0.02 μg/mL within about 3 to about 60 minutes (e.g., about: 60, 50, 40, 30, 20, 15, 10, 5, or 3 minutes), or about 10 to about 15 minutes (e.g., about: 10, 11, 12, 13, 14, or 15 minutes) of intranasal administration. In one embodiment, the compositions herein increase the blood concentration of an anti-anaphylactic and/or anti-anaphylactoid agent (e.g., epinephrine) by about 0.01 to 0.04 μg/mL, for example 0.02 or 0.03 μg/mL, in about 10-15 minutes (e.g., about: 10, 11, 12, 13, 14, or 15 minutes), or about 3 to about 60 minutes (e.g., about: 60, 50, 40, 30, 20, 15, 10, 5, or 3 minutes).

In another aspect, provided herein is an intranasal dry powder composition comprising a vasoactive agent. In some embodiments, the vasoactive agent is epinephrine or a pharmaceutically acceptable salt thereof. In some embodiments, the vasoactive agent is vasopressin or a pharmaceutically acceptable salt thereof. In some embodiments, the vasoactive agent is atropine or a pharmaceutically acceptable salt thereof. In some embodiments, the vasoactive agent does not comprise cocaine or a derivative thereof. In some embodiments, a single dose of the vasoactive agent is about 0.01 mg to about 10 mg. In another aspect, the amount of a vasoactive agent (e.g., epinephrine) can be at least about: 0.01 mg, 0.05 mg, 1.0 mg, 2.0 mg, 5.0 mg, or 10 mg in the compositions. In some embodiments, the vasoactive agent (e.g., epinephrine) present in the compositions can be about: 0.01 mg to 0.05 mg, 0.05 mg to 0.75 mg, 0.75 mg to 1.5 mg, 1.5 mg to 3.0 mg, 3.0 to 4.5 mg, 4.5 to 6.0 mg, 6.0 to 7.5 mg, 7.5 to 9.0 mg, or 9.0 to 10.0 mg. In some embodiments, the amount of a vasoactive agent (e.g., epinephrine) can be about: 0.15, 0.3, 0.5, 0.75, 1, 1.5, 2, 3, 4, 5, 6, 7, 8, 9 or 10 mg in the compositions. In one instance, a single dose of the vasoactive agent is about 0.75 mg. In one instance, a single dose of the vasoactive agent is about 1.5 mg. In another instance, a single dose of the vasoactive agent is about 3.0 mg. In another related aspect, the dose of a vasoactive agent (e.g., epinephrine) can be adjusted according to the weight of the patient at an increment of at least 0.01 mg/kg, or one wherein the dose can be repeated a number of times if the patient failed to increase the patient's arterial pressure. In some embodiments, the composition, in the form of a single dose, contains about 0.01 mg to about 10 mg of vasoactive agent. In some embodiments, the composition, in the form of a single dose, contains about 0.75 mg, 1.5 mg, or 3.0 mg of the vasoactive agent.

A unit dosage herein can range from about 0.01 mg to about 1 mg, for example about: 0.1, 0.2, 0.3, 0.4, 0.5, 0.6, 0.7, 0.8, 0.9, or 1 mg, of a composition. A unit dosage can also be at least about: 0.01 0.1, 0.5, or 1 mg, of a composition. Administration of the compositions herein can be repeated, e.g., every 5-20 minutes as necessary. In some embodiments, antihistamines (e.g., H₁ and/or H₂ receptor antagonists) and/or corticosteroids can be used in conjunction with a vasoactive agent (e.g., epinephrine), a formulation or a unit dose containing thereof, for managing patients suffering from cardiac arrest and/or hypotensive shock.

In some embodiments, the vasoactive agent is about 0.25% to about 50% w/w of the weight of the composition, for example about: 0.25%, 0.5%, 1%, 2%, 3%, 4%, 5%, 6%, 7%, 7.5%, 8%, 9%, 10%, 11%, 12%, 13%, 14%, 15%, 16%, 17%, 18%, 19%, 20%, 25%, 30%, 35%, 40%, 45%, or 50% w/w, based on the weight of the formulations and/or dosage units. For example, the vasoactive agent can be about 4%, about 7.5%, or about 15% w/w of the weight of the composition. In some embodiments, a vasoactive agent (e.g., epinephrine) can be present in an amount of at least about: 0.25% w/w, 1% w/w, 5% w/w, 10% w/w, 20% w/w, 30% w/w, 40% w/w, or 50% w/w based on the weight of the formulations and and/or dosage units. In some embodiments, a vasoactive agent (e.g., epinephrine) can be present in an amount of about: 0.25% to 1% w/w, 1% to 5% w/w, 5% to 10% w/w, 10% to 20% w/w, 20% to 30% w/w, 30% to 40% w/w, or 40% to 50% w/w based on the weight of the formulations and/or dosage units.

In some embodiments, the dry powder composition disclosed herein when administered to a patient, produces a maximal blood concentration (Cₘₐₓ) of a vasoactive agent (e.g., epinephrine) which can be at least about: 2- to 3-fold, 3- to 5-fold, 5- to 7-fold, or 7- to 10-fold more than the baseline level of the vasoactive agent in the patient. In some embodiments, the dry powder composition when administered to a patient, produces a maximal blood concentration (Cₘₐₓ) of the vasoactive agent at least 2-, 3-, 4-, 5-, 6-, 7-, 8-, 9-, or 10-fold more than the baseline level of the vasoactive agent in the patient. In one embodiment, the dry powder composition when administered to a patient, produces a maximal blood concentration (Cₘₐₓ) of the vasoactive agent at least 2 fold more than the baseline level of the vasoactive agent in the patient. In one embodiment, the compositions herein may increase the blood concentration of a vasoactive agent (e.g., epinephrine) by about 0.01 to 0.1 μg/mL. In one embodiment, the compositions herein may increase the blood concentration of a vasoactive agent (e.g., epinephrine) by about 0.01, 0.02, 0.03, 0.04, 0.05, 0.06, 0.07, 0.08, 0.09, or 0.1 μg/mL.

In some embodiments, the dry powder composition disclosed herein when administered to a patient, reaches a maximal blood concentration of a vasoactive agent (e.g., epinephrine) in less than about 60 minutes (Tₘₐₓ) after administration. In some embodiments, the dry powder composition when administered to a patient, reaches a maximal blood concentration (Tₘₐₓ) of the vasoactive agent in less than about 60, 50, 40, 30, 20, 15, 10, 5, or 3 minutes (Tₘₐₓ) after administration. In one embodiment, the dry powder composition when administered to a patient, reaches a maximal blood concentration (Tₘₐₓ) of the vasoactive agent in less than about 20 minutes after administration. In some embodiments, the dry powder composition when administered to a patient, reaches a mean AUC_{(0-180 minutes)} of the vasoactive agent which is at least 20%, 30%, 40% 50%, 60%, 70%, 80%, 90%, 100%, 110%, 120%, 130%, 140% or 150% of the mean AUC_{(0-180 minutes)} of IV, an IM, or SQ injected vasoactive agent. In some embodiments, the dry powder composition when administered to a patient, reaches a mean AUC_{(0-inf)} of the vasoactive agent which is at least 20%, 30%, 40% 50%, 60%, 70%, 80%, 90%, 100%, 110%, 120%, 130%, 140% or 150% of the mean AUC_{(0-inf)} of an IV, IM, or SQ injected vasoactive agent. In some embodiments, the IV, IM, or SQ injected vasoactive agent contains 0.10 mg, 0.15 mg, 0.20 mg, 0.25 mg, 0.30 mg, 0.35 mg, 0.40 mg, 0.45 mg, 0.50 mg, 0.55 mg, 0.60 mg, 0.65 mg, 0.70 mg, 0.75 mg, 0.80 mg, 0.85 mg, 0.90 mg, 0.95 mg or 1.0 mg of the vasoactive agent. For example, the dry powder composition when administered to a patient, reaches a mean AUC_{(0-180 minutes)} of the vasoactive agent which is at least 80% of the mean AUC_{(0-180 minutes)} of a 0.15 mg IV injected vasoactive agent. In another instance, the dry powder composition when administered to a patient, reaches a mean AUC_{(0-inf)} of the vasoactive agent which is at least 80% of the mean AUC_{(0-inf)} of a 0.15 mg IV injected vasoactive agent. In some embodiments, the IV, IM, or SQ injected a vasoactive agent is epinephrine injected by EpiPen™ autoinjector. In some embodiments, the dry powder composition when administered to a patient, reaches a mean AUC_{(0-180 minutes)} of the vasoactive agent which is at least 100,000 pg·min/mL, 200,000 pg·min/mL, 300,000 pg·min/mL, 400,000 pg·min/mL, 500,000 pg·min/mL, 600,000 pg·min/mL, 700,000 pg·min/mL, 800,000 pg·min/mL, 900,000 pg·min/mL, 1,000,000 pg·min/mL. In some embodiments, the dry powder composition when administered to a patient, reaches a mean AUC_{(0-inf)} of the vasoactive agent which is at least 100,000 pg·min/mL, 200,000 pg·min/mL, 300,000 pg·min/mL, 400,000 pg·min/mL, 500,000 pg·min/mL, 600,000 pg·min/mL, 700,000 pg·min/mL, 800,000 pg·min/mL, 900,000 pg·min/mL, 1,000,000 pg·min/mL, 1,200,000 pg·min/mL, 1,400,000 pg·min/mL, 1,600,000 pg·min/mL, 1,800,000 pg·min/mL, 2,000,000 pg·min/mL.

In certain embodiments, the dry powder compositions and/or dosage units herein can raise the blood concentration of a vasoactive agent (e.g., epinephrine) to about 0.02 μg/mL within about 3 to about 60 minutes (e.g., about: 60, 50, 40, 30, 20, 15, 10, 5, or 3 minutes), or about 10 to about 15 minutes (e.g., about: 10, 11, 12, 13, 14, or 15 minutes) of intranasal administration. In one embodiment, the compositions herein increase the blood concentration of a vasoactive agent (e.g., epinephrine) by about 0.01 to 0.04 μg/mL, for example 0.02 or 0.03 μg/mL, in about 10-15 minutes (e.g., about: 10, 11, 12, 13, 14, or 15 minutes), or about 3 to about 60 minutes (e.g., about: 60, 50, 40, 30, 20, 15, 10, 5, or 3 minutes).

In another aspect, a single dose of epinephrine in the dry powder compositions and/or dosage units given intranasally can be bioequivalent (for example, in terms of peripheral blood levels, systemic exposure of epinephrine) to intravenously (IV), intramuscularly (IM) or subcutaneously (SQ) injected epinephrine (e.g., using EpiPen™ autoinjector of 0.15 mg for pediatric patients and 0.3 mg for adult patients). For example, bioequivalence can be that 90% confidence interval of a mean Tₘₐₓ (e.g., the time to reach maximal blood concentration), a mean Cₘₐₓ (e.g., maximal blood concentration), a mean AUC₍₀₋ₜ₎ (e.g., area under the plasma/serum/blood concentration-time curve from time zero to time t), and/or a mean AUC_{(0-∞)} (e.g., area under the plasma/serum/blood concentration-time curve from time zero to time infinity) of the test to reference are within 80.00% to 125.00%, optionally, in the fasting state.

In a further embodiment, the intranasal dry powder compositions can be present in amounts of up to 100 mg, for example about: 1 to 5 mg, 5 to 10 mg, 10 to 20 mg, 20 to 40 mg, 40 to 60 mg, 60 to 80 mg, or 80 to 100 mg. In some embodiments, the compositions herein can be present in about: 1, 5, 10, 15, 20, 25, 30, 35, 40, 45, or 50 mg.

In another aspect, the dry powder composition may further comprise a vasodilator. In another embodiment, the vasodilator is phentolamine or a pharmaceutically acceptable salt thereof. In some embodiments, a single dose of the vasodilator is about 0.01 mg to about 10 mg. In some embodiments, a vasodilator (e.g., phentolamine) herein can be present in the compositions in about: 0.001 mg to 0.01 mg, 0.01 mg to 0.05 mg, 0.05 to 0.1 mg, 0.1 to 0.5 mg, 0.5 to 0.75 mg, 0.75 mg to 1.5 mg, 1.5 mg to 3.0 mg, 3.0 to 4.5 mg, 4.5 to 6.0 mg, 6.0 to 7.5 mg, 7.5 to 9.0 mg, or 9.0 to 10.0 mg. In some embodiments, a vasodilator (e.g., phentolamine) herein can be at least about: 0.001 mg, 0.01 mg, 0.1 mg, 0.5 mg, 1.0 mg, 2.0 mg, 5.0 mg, or 10.0 mg in the compositions and/or dosage units. In some embodiments, a single dose of the vasodilator is about 0.5 mg or about 1.0 mg. In some embodiments, the composition, in the form of a single dose, contains about 0.01 mg to about 10 mg of the vasodilator. In some embodiments, the composition, in the form of a single dose, contains about 0.5 mg or about 1.0 mg of the vasodilator agent.

In some embodiments, the amount of the vasodilator is about 0.005% to about 50% w/w of the weight of the composition, for example about: 0.005%, 0.01%, 0.05%, 0.1%, 0.5%, 1%, 2%, 2.5%, 3%, 4%, 5%, 6%, 7%, 8%, 9%, 10%, 15%, 20%, 25%, 30%, 35%, 40%, 45%, or 50% w/w of the weight of the composition. In some embodiments, the amount of a vasoactive agent, and/or an anti-anaphylactic and/or anti-anaphylactoid agent (e.g., epinephrine) can be at least about: 0.005% w/w, 0.05% w/w, 0.5% w/w, 1% w/w, 2% w/w, 2.5% w/w, 5% w/w, 10% w/w, 20% w/w, 30% w/w, 40% w/w, or 50% w/w based on the weight of the composition. In some embodiments, the amount of the vasodilator is about 2.5% w/w of the weight of the composition.

In some embodiments, the dry powder composition further comprises a pharmaceutically acceptable carrier. In some embodiments, the carrier further comprises a first cellulose. In some embodiments, the first cellulose is a crystalline cellulose. In some embodiments, the first cellulose is a microcrystalline cellulose. In some embodiments, the first cellulose has an average particle diameter of about 100 μm or less, for example about: 90 to 100 μm, 80 to 90 μm, 70 to 80 μm, 60 to 70 μm, 50 to 60 μm, 40 to 50 μm, 30 to 40 μm, 20 to 30 μm, or 10 to 20 μm. In some embodiments, the first cellulose has an average particle diameter of less than about 100 μm, 90 μm, 80 μm, 70 μm, 60 μm, 50 μm, 40 μm, 30 μm, 20 μm, 10 μm, or 5 μm. In some embodiments, the first cellulose has an average particle diameter of about 30 μm or less. In some embodiments, the dry powder composition further comprises a second cellulose. In some embodiments, the second cellulose is a crystalline cellulose. In some embodiments, the second cellulose is a microcrystalline cellulose. In some embodiments, the dry powder composition may further comprise starch. In some embodiments, the dry powder composition may further comprise a second cellulose and starch. In some embodiments, the second cellulose and/or starch have an average particle diameter of about 30 to about 100 μm, for example about: 30-40 μm, 30-50 μm, 30-60 μm, 30-70 μm, 30-80 μm, or 30-90 μm. In some embodiments, the second cellulose and/or starch has an average particle diameter of less than about 100 μm, 90 μm, 80 μm, 70 μm, 60 μm, 50 μm, 40 μm, 30 μm, 20 μm, 10 μm, or 5 μm. In some embodiments, the second cellulose, the starch, or the second cellulose and starch each individually has an average particle diameter of about 30 to about 100 p.m.

In one aspect, an average particle diameter of a dry powder composition is determined using a laser-diffraction particle size distribution analyzer. In some embodiments, an average particle diameter of a dry powder composition is determined using sieve sorting.

In one aspect, the carrier may further comprise an excipient. In some embodiments, the carrier may further comprise an anticaking agent. In some embodiments, the anticaking agent further comprises tribasic calcium phosphate. In some embodiments, the excipient is about 0.5% to about 5% w/w of the weight of the composition. In some embodiments, the tribasic calcium phosphate is about 0.5% to about 5% w/w of the weight of the composition. In some embodiments, the tribasic calcium phosphate has an average particle diameter of about 100 μm or less, for example about: 90 to 100 μm, 80 to 90 μm, 70 to 80 μm, 60 to 70 μm, 50 to 60 μm, 40 to 50 μm, 30 to 40 μm, 20 to 30 μm, or 10 to 20 μm. In some embodiments, the tribasic calcium phosphate has an average particle diameter of about 30 μm to 100 μm.

In an embodiment, the pharmaceutically acceptable carrier present in the intranasal dry powder compositions can be a mixture of first microcrystalline cellulose, second microcrystalline cellulose or a starch, and tribasic calcium phosphate (see U.S. Pat. No. 8,337,817).

In one aspect, the pharmaceutically acceptable carrier can comprise: i) a first crystalline cellulose with an average particle diameter of about 30 μm or less, for example about: 30-10 μm, 30-15 μm, 30-20 μm, or 30-25 μm; ii) tribasic calcium phosphate; and iii) a second crystalline cellulose, or starch, with an average particle diameter of about 30 to about 100 μm, for example about: 30-40 μm, 30-50 μm, 30-60 μm, 30-70 μm, 30-80 μm, or 30-90 μm.

The vasoactive agent, and/or anti-anaphylactic and/or anti-anaphylactoid agent (e.g., epinephrine), vasodilator (e.g., phentolamine) and other enabling agents can be individually substantially amorphous or crystalline. In some embodiments, the compositions and/or dosage units herein can be in the form of particles, and the shapes of the particles can be individually, e.g., cylindrical, discoidal, spherical, tabular, ellipsoidal, angular, and/or irregular.

In some embodiments, the average particle diameter of the vasoactive agent, and/or anti-anaphylactic and/or anti-anaphylactoid agent (e.g., epinephrine), vasodilator (e.g., phentolamine) and carrier can be, individually, up to 100 μm, up to 50 μm, or up to 30 μm, for example about: 10-20, 20-30, 30-40, 40-50, 50-60, 60-70, 70-80, 80-90, or 90-100 μm; or about: 10, 15, 20, 25, 30, 35, 40, 45, 50, 55, 60, 65, 70, 75, 80, 85, 90, 95, or 100 μm.

In some embodiments, the median particle diameter of the vasoactive agent, and/or anti-anaphylactic and/or anti-anaphylactoid agent (e.g., epinephrine) powder herein can be about 30 μm, for example 28.7 μm. In some embodiments, the median particle diameter of the vasoactive agent, and/or anti-anaphylactic and/or anti-anaphylactoid agent (e.g., epinephrine) powder herein can be about: 10-50, 20-40, or 25-35 μm. In one aspect, 90% of the vasoactive agent, and/or anti-anaphylactic and/or anti-anaphylactoid agent (e.g., epinephrine) particles herein can have a particle diameter under about 50 μm, for example about 45.5 μm, or about: 40, 45, 35, 30, 25, or 20 μm. In another aspect, about 10% of the vasoactive agent, and/or anti-anaphylactic and/or anti-anaphylactoid agent (e.g., epinephrine) particles herein can have a particle diameter under about 20 μm, for example about 17.3 μm, or about: 19, 18, 17, 16, 15, 14, 13, 12, 11, or 10 μm.

In addition to a vasoactive agent, and/or an anti-anaphylactic and/or anti-anaphylactoid agent (e.g., epinephrine) and a vasodilator, the intranasal dry powder compositions and/or dosage units herein can comprise one or more enabling agents, for example, epinephrine potentiators, reversible COMT inhibitors, mucosal permeation enhancers, agents that reduce mucosal transit time, agents that increase mucosal absorption or adhesion or transport, surfactants, chelators, pharmaceutically acceptable excipients, non-sulfite stabilizers, preservatives, thickening agents, humectants, antihistamines, solubilizing agents, taste and smell masking agents, antioxidant enzymes, viscosity enhancing agents, dispersing agents, colorants, or any combination thereof. In some embodiments, the dry powder composition further comprises a COMT inhibitor. In some embodiments, the COMT inhibitor further comprises a reversible COMT inhibitor. In a certain embodiment, the reversible COMT inhibitor is entacapone or a pharmaceutically acceptable salt thereof.

In another aspect, provided herein is a kit comprising: (a) a dose of an intranasal dry powder composition disclosed herein; (b) instructions reciting when the dry powder composition in (a) is to be administered to a subject. In some embodiments, the kit may further comprise an intranasal delivery apparatus for dispensing the dry powder composition. In some embodiments, the apparatus delivers a therapeutically acceptable amount of the dry powder composition. In some embodiments, the apparatus may intranasally deliver a therapeutically acceptable amount of the dry powder composition. In some embodiments, the dry powder composition is delivered intranasally. In some embodiments, the apparatus further comprises a reservoir that holds the dry powder composition. The apparatus can comprise a reservoir and means for expelling the pharmaceutical dose in the form of a spray, wherein a quantity of the pharmaceutical dose can be contained within the reservoir. In some embodiments, the apparatus is disposable. In some embodiments, the apparatus is reusable. In some embodiments, the apparatus is recyclable. In some embodiments, the package comprises one reservoir, wherein the reservoir contains multiple doses of the dry powder composition. In some embodiments, the package further comprises one intranasal delivery apparatus.

In one aspect, the kit herein may further comprise an automated external defibrillator (AED) system. In some embodiments, the instructions for use of the AED advise the user on administration of doses of nasal epinephrine to the patient in conjunction with use of the AED device. In some embodiments, the instructions in the kit further recite how to operate the automated external defibrillator (AED) system. In some instances, the instructions are pre-loaded on the automated external defibrillator (AED) system. In some embodiments, the automated external defibrillator (AED) system contains a self-contained power source. In some embodiments, the self-contained power source is a battery. In some embodiments, the dry powder composition in the kit has a weight of less than 5, 10, 15, 20, 25, 30, 35, 40, 45, 50, 55, 60, 65, 70, 75, 80, 85, 90, 95, or 100 lbs. In some embodiments, the battery is rechargeable. In some embodiments, the dry powder composition in the kit has a weight of less than 20 lbs.

In some embodiments, the device can be programmed to dispense one or more pharmaceutical doses. The nasal spray can be designed for discharge of multiple spray doses, e.g., 1, 2, 3, 4, 5, 6, 7, 8, 9, 10 or more. It can be designed to administer the intended dose with multiple sprays, e.g., two sprays, one in each nostril or in one nostril, or as a single spray, or to vary the dose in accordance with the body weight or maturity of the patient. The object of the design of the safety spray device can be to assure to the extent possible that a consistent loading dose of a vasoactive agent, and/or an anti-anaphylactic and/or anti-anaphylactoid agent (e.g., epinephrine), which can be the blood equivalent of IM administered dose (0.15 mg in pediatric subjects and 0.3 mg in adults), can be delivered to the bloodstream to a subject, for example subcutaneously or intramuscularly.

In one aspect, the dry powder composition may be a multi-unit package. In some embodiments, the package comprises multiple reservoirs, wherein each reservoir contains a single dose of the dry powder composition. In some embodiments, the package comprises one apparatus and multiple reservoirs, wherein each reservoir contains a single dose of the dry powder composition. In some embodiments, the package comprises multiple apparatuses, wherein each apparatus contains one reservoir which contains a single dose of the dry powder composition. In an embodiment, the apparatus can comprise a pump spray device in which the means for expelling a single or multiple doses can comprise a metering pump, or can be a sterile single dose disposable device. The dose to be delivered can be metered by the spray pump, which can be finger or hand-actuated. The apparatus herein can be a single-use device or a multiple-use device. The single-use device can be preloaded with a drug formulation and disposed of after use. The multiple-use device can accept encapsulated formulations with negligible residue build-up even after high usage.

In one aspect, the multi-unit package herein may allow for easy and quick visual verification of units used. In some embodiments, the package is labeled for easy and quick visual verification of units used. In some embodiments, the package is color labeled for easy and quick visual verification of units used. In some embodiments, the package is labeled for easy and quick visual verification of the number of units which have been removed and used, and by extension the amount of drug that has been delivered to the patient.

In a further aspect, the compositions herein can also be administered using a nasal metered dose spray, a metered dose inhaler, or a measured dose inhaler.

In one aspect, the formulations or dosage units herein are not or do not comprise spray-dried particles. In some embodiments, the formulations herein do not possess a fine particle fraction of less than 5.6 microns of at least about 45 percent. In some embodiments, the formulations herein do not comprise particles comprising: (a) about 11 to about 21 weight percent epinephrine bitartrate; (b) about 62 to about 82 weight percent leucine; and/or (c) about 7 to about 17 weight percent sodium tartrate.

Provided herein are methods of treating a patient by intranasally administrating the dry powder composition disclosed herein. Also provided herein are methods of treating a patient by using the kit disclosed herein.

The methods, kits, compositions doses or products herein are useful for treating patients. In some embodiments, the patient has minimal or no cardiac activity. In some embodiments, the patient has low blood pressure. In some embodiments, the patient has hypotension. In some embodiments, the patient is experiencing hypotensive shock. In some embodiments, the hypotensive shock is secondary to causes comprising trauma, hypovolemia, bradycardia, and/or septic shock. In some embodiments, the patient is experiencing an allergic reaction. In some embodiments, the patient is experiencing anaphylaxis. In some embodiments, the composition can also provide a fast onset time and can be suitable for intranasal use.

In some embodiments, the intranasal dry powder composition is sufficient to increase the arterial pressure in the patient within 60 minutes, 50 minutes, 40 minutes, 30 minutes, 20 minutes, 10 minutes, 5 minutes, 3 minutes, 2 minutes, or 1 minutes after administration. In some embodiments, the intranasal dry powder composition is sufficient to increase the mean arterial pressure in the patient within 60 minutes, 50 minutes, 40 minutes, 30 minutes, 20 minutes, 10 minutes, 5 minutes, 3 minutes, 2 minutes, or 1 minutes after administration. In some embodiments, the intranasal dry powder composition is sufficient to increase coronary perfusion pressure in the patient within 60 minutes, 50 minutes, 40 minutes, 30 minutes, 20 minutes, 10 minutes, 5 minutes, 3 minutes, 2 minutes, or 1 minutes after administration. In some embodiments, the intranasal dry powder composition is sufficient to resume a spontaneous circulation in the patient within 60 minutes, 50 minutes, 40 minutes, 30 minutes, 20 minutes, 10 minutes, 5 minutes, 3 minutes, 2 minutes, or 1 minutes after administration. In some embodiments, the intranasal dry powder composition is sufficient to relieve the allergic reaction in the patient within 60 minutes, 50 minutes, 40 minutes, 30 minutes, 20 minutes, 10 minutes, 5 minutes, 3 minutes, 2 minutes, or 1 minutes after administration.

Also provided herein are methods for treating anaphylaxis, anaphylactoid reactions, bronchospasm, cardiac arrest, hypotensive shock, or other situations requiring the need to implement cardiopulmonary resuscitation (CPR) and/or basic or advanced cardiac life support (ACLS) in an individual, comprising, applying to a mucosal surface(s) of the nasal cavity or cavities of an individual (e.g., the mucosal surfaces of the anterior regions of the nose, the frontal sinus, the maxillary sinuses, and/or on each of the mucosal surfaces which overlie the turbinates covering the conchas) any of the pharmaceutical compositions or dosage units herein by administering a nasal vasoactive agent, and/or anti-anaphylactic and/or anti-anaphylactoid agent (e.g., epinephrine) loading dose (e.g., the amount of epinephrine administered nasally which results in the systemic blood bioequivalent of intravenously (IV), intramuscularly (IM) or subcutaneously (SQ) administered epinephrine for example, for the 0.15 mg and 0.3 mg doses of EpiPen™). In a related aspect, the method of treating a patient with anaphylaxis, anaphylactoid reactions, cardiac arrest, and/or bronchospasm in need of treatment from a nasal loading dose of about 0.05 mg to about 10 mg of a vasoactive agent, and/or an anti-anaphylactic and/or anti-anaphylactoid agent (e.g., epinephrine), for example about 0.5 to about 5 mg, or about: 0.75, 1.5, or 3.0 mg; 0.001 mg (or 1 μg) to 10 mg of a vasodilator (e.g., phentolamine), for example about 0.1 to about 5 mg, about 0.1 to about 1 mg, or about 0.5 mg; a pharmaceutically acceptable carrier mixture of about 1 to about 50 mg, for example about 10 to about 30 mg, about 15 to about 20 mg, or about 18 mg, and optionally, an agent that reduces mucosal transit time, an agent that increases mucosal absorption and/or adhesion, an agent that enhances mucosal transport, (or the enantiomers, diastereoisomers, racemates, and the salts of such compounds with pharmaceutically acceptable counterions), wherein the amounts can be synergistic for the treatment of anaphylaxis, anaphylactoid reactions, bronchospasm, and/or cardiac arrest. When used in such low doses, compositions herein can provide a sufficiently high peak blood plasma concentration of a vasoactive agent, and/or an anti-anaphylactic and/or anti-anaphylactoid agent (e.g., epinephrine), at least about: 2-fold, 3-5 fold, 5-7 fold, or 7-10 fold more than baseline levels rapidly after administration, within about: 60, 50, 40, 30, 20, 15, 10, 5, or 3 minutes, to be effective in the treatment or reducing the symptoms of anaphylaxis, anaphylactoid reactions, bronchospasm, and/or cardiac arrest.

In yet another aspect, provided herein is a method of nasal delivery that employs a single use sterile premixed formulation (dry powder or aqueous) containing a vasoactive agent, and/or an anti-anaphylactic and/or anti-anaphylactoid agent (e.g., epinephrine) and/or other agents herein that can be disposed of after use.

In some embodiments, the method herein further comprises (a) initiating cardiopulmonary resuscitation (CPR), (b) using an automated external defibrillator (AED), or both (a) and (b). In some embodiments, the intranasal dry powder composition is administered if (a), (b), or both fail to increase the arterial pressure in the patient within 60 minutes, 50 minutes, 40 minutes, 30 minutes, 20 minutes, 10 minutes, 5 minutes, 3 minutes, 2 minutes, or 1 minutes after administration. In some embodiments, the intranasal dry powder composition is administered if (a), (b), or both fail to increase the mean arterial pressure in the patient within 60 minutes, 50 minutes, 40 minutes, 30 minutes, 20 minutes, 10 minutes, 5 minutes, 3 minutes, 2 minutes, or 1 minutes after administration. In some embodiments, the intranasal dry powder composition is administered if (a), (b), or both fail to increase coronary perfusion pressure in the patient within 60 minutes, 50 minutes, 40 minutes, 30 minutes, 20 minutes, 10 minutes, 5 minutes, 3 minutes, 2 minutes, or 1 minutes after administration. In some embodiments, the intranasal dry powder composition is administered if (a), (b), or both fail to resume a spontaneous circulation in the patient within 60 minutes, 50 minutes, 40 minutes, 30 minutes, 20 minutes, 10 minutes, 5 minutes, 3 minutes, 2 minutes, or 1 minutes after administration. In some embodiments, the intranasal dry powder composition is administered if (a), (b), or both fail to relieve the allergic reaction in the patient within 60 minutes, 50 minutes, 40 minutes, 30 minutes, 20 minutes, 10 minutes, 5 minutes, 3 minutes, 2 minutes, or 1 minutes after administration.

In another aspect, the methods, kits, compositions doses or products herein may be useful for treating patients in a hospital. In some embodiments, the patient is not in a hospital. In some embodiments, the patient is in a hospital. In some embodiments, the patient is in a combat setting. In some embodiments, the patient is in a civil emergency setting. In some embodiments, the patient has a wound.

In some embodiments, the method of dilating a bronchus in a subject comprises the intranasal administration of the dry powder composition disclosed herein. In some embodiments, the dilation occurs without substantial pulmonary inhalation. In some embodiments, the method of delivering epinephrine in a subject at least to one of alpha adrenergic receptors, beta adrenergic receptors, or any combination thereof, comprises the intranasal administration of the dry powder composition disclosed herein. In some embodiments, the alpha adrenergic receptors consist of the group including alpha-1 and alpha-2 adrenergic receptors. In some embodiments, the beta adrenergic receptors consist of the group including beta-1, beta-2 and beta-3 adrenergic receptors. In some embodiments, the delivering of epinephrine is localized. In some embodiments, the delivering of epinephrine is systematic. In some embodiments, the method of treating a subject with asthma comprises the intranasal administration of the dry powder composition disclosed herein. In some embodiments, the method of treating a subject with croup comprises the intranasal administration of the dry powder composition disclosed herein. In some embodiments, the method of treating a condition in a subject by stimulating at least one of alpha adrenergic receptors, beta adrenergic receptors, or any combination thereof, comprising the intranasal administration of the dry powder composition disclosed herein. In some embodiments, the method of treating a subject by increasing the heart rate of the subject comprises the intranasal administration of the dry powder composition disclosed herein. In some embodiments, the method of treating a subject by i increasing the respiratory rate of the subject comprises the intranasal administration of the dry powder composition disclosed herein. In some embodiments, the method of increasing the blood concentration of epinephrine in a subject comprises the intranasal administration of the dry powder composition disclosed herein. In some embodiments, the method of in treating urticaria in a subject comprises the intranasal administration of the dry powder composition disclosed herein. In some embodiments, the method of in treating pulmonary edema in a subject comprises the intranasal administration of the dry powder composition disclosed herein. In some embodiments, the method of in treating serum sickness in a subject in a subject comprises the intranasal administration of the dry powder composition disclosed herein. In some embodiments, the method of in treating a subject with anaphylaxis resulted from an insect bite comprises the intranasal administration of the dry powder composition disclosed herein. In some embodiments, the method of in treating a subject with anaphylaxis resulted from ingested food comprises the intranasal administration of the dry powder composition disclosed herein. In some embodiments, the method of in treating a subject with anaphylaxis resulted from a drug reaction comprises the intranasal administration of the dry powder composition disclosed herein. In some embodiments, the method of treating itching in a subject comprises the intranasal administration of the dry powder composition disclosed herein. In some embodiments, the method of in treating a subject with snake bite comprises the intranasal administration of the dry powder composition disclosed herein. In some embodiments, the method of counteracting bronchoconstriction effects in a subject following certain chemical exposures comprises the intranasal administration of the dry powder composition disclosed herein.

In another aspect, the subject of the treatment can be human.

### BRIEF DESCRIPTION OF THE DRAWINGS

FIG. 1 is the critical actions needed to improve chances of survival of an out-of hospital cardiac arrest (OHCA)

FIG. 2 is the automated external defibrillation algorithm incorporating intranasal epinephrine product.

FIG. 3 is the modified protocol of incorporating intranasal epinephrine product to improve chances of survival of an out-of hospital cardiac arrest (OHCA).

FIG. 4A is a line chart showing time profiles of mean plasma epinephrine concentration within the first 3 hours after administration to the test monkeys IN μco™ Preparation Placebo, IM Placebo, IM Solution 0.15 mg, IN μco™ Preparation 0.75 mg, IN μco™ Preparation 3.0 mg, IN μco™ Preparation 1.5 mg Containing Caffeine (both nostrils), and IN μco™ Preparation 1.5 mg Containing Phentolamine (both nostrils), respectively.

FIG. 4B is a line chart showing time profiles of mean plasma epinephrine concentration within the first 30 minutes after administration to the test monkeys IN μco™ Preparation Placebo, IM Placebo, IM Solution 0.15 mg, IN μco™ Preparation 0.75 mg, IN μco™ Preparation 3.0 mg, and IN μco™ Preparation 1.5 mg Containing Phentolamine (both nostrils), respectively.

FIG. 4C is a bar chart showing mean plasma epinephrine AUC₀₋₁₀ values within the first 10 minutes after administration to the test monkeys IM Solution 0.15 mg, IN μco™ Preparation 1.5 mg Containing Phentolamine (both nostrils), IN μco™ Preparation 3.0 mg, IN μco™ Preparation 0.75 mg, IN μco™ Preparation Placebo, and IM Placebo, respectively.

FIG. 5 is a line chart showing the primary particle size distribution of the test epinephrine powder on each dispersion pressure.

FIG. 6 is a line chart showing the trend of primary particle size distribution of the test epinephrine powder on each dispersion pressure.

FIG. 7 is a line chart showing comparison of primary particle size distribution of the test epinephrine powder between Dry Dispersion Method (0.5 bar) and Wet Dispersion Method.

FIGS. 8A-8D are HPLC-UV chromatograms of the test epinephrine formulations in Experiment I: 5A) Specificity solution BA1; 5B) Specificity solution BA2; 5C) Specificity solution BA3; and 5D) Standard solution S-3.

FIG. 9 is a line chart showing linearity of HPLC-US assay in Experiment I for epinephrine in intranasal powder formulations.

FIGS. 10A-10B are HPLC-UV chromatograms of the test epinephrine formulations in Experiment II: 10A) Specificity solution BA1; and 10B) Standard solution S-3.

FIG. 11 is a line chart showing linearity of HPLC-US assay in Experiment II for epinephrine in intranasal powder formulations.

### INCORPORATION BY REFERENCE

All publications, patents, and patent applications herein are incorporated by reference to the same extent as if each individual publication, patent, or patent application was specifically and individually indicated to be incorporated by reference. In the event of a conflict between a term herein and a term in an incorporated reference, the term herein controls.

### DETAILED DESCRIPTIONS

Unless defined otherwise, all technical and scientific terms used herein have the same meaning as commonly understood by one of the ordinary skill in the art to which this invention belongs. Although any methods and materials similar or equivalent to those described herein can be used in the practice or testing of the formulations or unit doses herein, some methods and materials are now described. Unless mentioned otherwise, the techniques employed or contemplated herein are standard methodologies. The materials, methods and examples are illustrative only and not limiting.

The details of one or more inventive embodiments are set forth in the accompanying drawings, the claims, and the description herein. Other features, objects, and advantages of the inventive embodiments disclosed and contemplated herein can be combined with any other embodiment unless explicitly excluded.

Unless otherwise indicated, open terms for example “contain,” “containing,” “include,” “including,” and the like mean comprising.

The singular forms “a,” “an,” and “the” are used herein to include plural references unless the context clearly dictates otherwise. Accordingly, unless the contrary is indicated, the numerical parameters set forth in this application are approximations that may vary depending upon the desired properties sought to be obtained by the present invention.

Unless otherwise indicated, some embodiments herein contemplate numerical ranges. When a numerical range is provided, unless otherwise indicated, the range includes the range endpoints. Unless otherwise indicated, numerical ranges include all values and sub ranges therein as if explicitly written out.

Unless otherwise noted, ‘vasoactive agent’ can refer to substance(s) that cause either vasoconstriction or vasodilation of blood vessels. If it causes vasoconstriction, it will increase coronary perfusion pressure or mean arterial pressure. It can also include an agent or substance, which causes indirect stimulation of a nerve that causes either vasoconstriction or vasodilation of blood vessels. Vasoactive agent according to this invention can include epinephrine, vasopressin, atropine, or pharmaceutically acceptable salts thereof.

Unless otherwise indicated, ‘loading dose’ herein can refer to the actual amount of a vasoactive agent (e.g., epinephrine), and/or an anti-anaphylactic and/or anti-anaphylactoid agent (e.g., epinephrine) administered intranasally.

Unless otherwise indicated, ‘effective dose’ can refer to the anti-anaphylactic and/or anti-anaphylactoid agent (e.g., epinephrine) dose required to treat anaphylaxis or reduce anaphylactoid reactions or symptoms in a subject. ‘Effective dose’ can refer to the vasoactive agent (e.g., epinephrine) dose required to treat cardiac arrest or hypotensive shock or symptoms in a subject.

Unless otherwise indicated, ‘baseline levels’ can refer to concentrations of a vasoactive agent (e.g., epinephrine), and/or an anti-anaphylactic and/or anti-anaphylactoid agent (e.g., epinephrine) in blood before administration by intranasal or intramuscular methods.

Unless otherwise indicated, ‘active ingredients’ or ‘active agents’ can refer to at least vasoactive agents, anti-anaphylactic and/or anti-anaphylactoid agents, vasodilators, of the compositions herein.

Unless otherwise indicated, “primary particle diameter” can refer to the particle size distribution of a powder in its non-aggregated state. Primary particle diameter can be determined using a laser-diffraction particle size distribution analyzer. In some embodiments, the particle size analyzer can be Mastersizer 2000 manufactured by Malvern Instruments Limited.

Unless otherwise indicated, “median diameter” can refer to a diameter that divides particles into two groups of equal numbers: a group with greater diameters and a group with smaller diameters. A median diameter can be determined using a laser-diffraction particle size distribution analyzer and can correspond to 50% volume in a determined cumulative particle size distribution curve. In some embodiments, the particle size analyzer can be Mastersizer 2000 manufactured by Malvern Instruments Limited.

Unless otherwise indicated, “average particle diameter” can be determined based on the particle size distribution by a sorting method. Unless otherwise indicated, “untapped bulk density” can be measured based on the second method for determination of bulk and tapped densities, among the general tests described in Part I of Supplement I to Japanese Pharmacopoeia, Fourteenth Edition.

Unless otherwise indicated, “average particle diameter” can be determined based on the particle size distribution by a sorting method. An average particle diameter can correspond to 50 (W/W) % on a cumulative particle size distribution curve obtained by sorting 10 g of particles for ten minutes on a electromagnetic sieve shaker, using standard sieves (which meet Japanese Industrial Standards (JIS)) layered in the order of aperture sizes 38 45, 53, 75, 106, 180, and 300 μm, and weighing the sample that remained on each sieve. This procedure is based on the second method of particle size distribution test, among the general tests described in the Japanese Pharmacopoeia, Fourteenth Edition, Part I.

Unless otherwise indicated, “untapped bulk density” can be measured based on the second method for determination of bulk and tapped densities, among the general tests described in Part I of Supplement I to Japanese Pharmacopoeia, Fourteenth Edition. Specifically, the density can be determined by pouring the sample evenly from above into a cylindrical vessel with an inner diameter of 46 mm and a height of 110 mm (measured volume, 180 ml) through a 1000-μm JIS standard sieve, and weighing the sample after smoothly leveling off the top of the vessel.

Unless otherwise indicated, “specific surface area” can be determined by the second method for determination of specific surface area (BET method), among the general tests described in the Japanese Pharmacopoeia, Fourteenth Edition, Part I. Specifically, the specific surface area can be determined based on the BET formula from the amount of nitrogen molecules adsorbed onto the powder surface after six hours of pre-vacuation at a fixed temperature (77.35 Kelvin).

Unless otherwise indicated, “angle of repose” can refer to a slope angle that can maintain a pile of powder accumulated in a way that it does not spontaneously collapse when dropped in the gravitational field. The angle can be measured by a funnel flow method. For example, measurement by the funnel flow method calculates the slope for a pile of powder that has been freely dropped through a funnel onto a disc and piled on a horizontal plane, based on the diameter of the circular bottom plane and the height of the powder pile. The angle of repose varies depending on particle size, surface properties, and the like. In general, the angle tends to be greater as the particle diameter becomes smaller. The angle of repose can serve as an indicator for powder flowability, and a smaller angle of repose means higher powder flowability.

Unless otherwise indicated, the term anaphylaxis can refer to an acute, systemic allergic reaction that occurs after an individual has become sensitized to an antigen. It can be associated with the production of high levels of immunoglobulin E (IgE) antibodies and/or with the release of histamines, which can cause muscle contractions, constriction of the airways (bronchospasm), and vasodilation. Symptoms of anaphylactic and/or anaphylactoid reactions can include hives, generalized itching, nasal congestion, wheezing, difficulty breathing, cough, cyanosis, lightheadedness, dizziness, confusion, slurred speech, rapid pulse, palpitations, nausea and vomiting, abdominal pain or cramping, skin redness or inflammation, nasal flaring, intercostals retractions, etc. Possible complications of severe anaphylactic and/or anaphylactoid reactions can include airway blockage, cardiac arrest, respiratory arrest, and/or shock.

Unless otherwise indicated, anaphylactoid reactions can be non-IgE mediated. Non-IgE-mediated causes can include factors causing marked complement activation for example plasma proteins or compounds which act directly on the mast cell membrane, for example vancomycin, quinolone antibiotics, and/or radiographic contrast media.

Unless otherwise indicated, “enabling agents” can refer to compounds or agents that can act synergistically with a vasoactive agent (e.g., epinephrine) and/or an anti-anaphylactic and/or anti-anaphylactoid agent (e.g., epinephrine) to enhance or promote its action or absorption or adhesion in target tissue to cause amelioration of the anaphylactic symptoms in a subject. Accordingly, the enabling agents can include one or more of the following in any combination; a reversible COMT inhibitor, a vasodilator, an epinephrine potentiator, a permeation and/or mucosal absorption and/or transport enhancer, an agent that reduces mucosal transit time, a thickener, an antihistamine and/or others as described herein.

Unless otherwise indicated, compositions and formulations herein can be powdery.

Unless otherwise indicated, dry powder compositions herein can contain water in an amount from about 0% to about 15% w/w, for example 0-10%, 0-5%, or 0-1% w/w; or about: 1%, 2%, 3%, 4%, 5%, 6%, 7%, 8%, 9%, 10%, 11%, 12%, 13%, 14%, or 15% w/w, based on the weight of the composition.

Unless otherwise indicated, compositions herein can be present in a free base form or a salt form. When a compound is recited, its salt form is also contemplated. The suitable salt forms can include besylate, tosylate, mesylate, succinate, salicylate, acetate, carboxylate, sulphate, phosphate, maleate, fumarate, lactate, tartrate, citrate, gluconate, oxalate, naphthsylate, hydrochloride, hydrobromide, hydroiodide, and hydrofluoride.

Unless otherwise indicated, subjects or patients herein can be mammalian or human, male or female, and child or adult, and can be subjects or patients in treatment (e.g., in need thereof).

Unless otherwise indicated, anaphylaxis can include anaphylactoid, and anti-anaphylaxis can include anti-anaphylactoid.

### Anti-Anaphylactic or Anti-Anaphylactoid Agent

The methods, kits, compositions and systems disclosed herein may comprise an anti-anaphylactic and/or anti-anaphylactoid agent. In some embodiments, the anti-anaphylactic and/or anti-anaphylactoid agent can be epinephrine or a pharmaceutically acceptable salt thereof. For example, the anti-anaphylactic and/or anti-anaphylactoid agent can be epinephrine hydrochloride, epinephrine free base, epinephrine maleate, epinephrine bitartrate, epinephrine methyl ester or hydrochloride, glycosyl epinephrine derivatives, dipavalyl epinephrine derivatives including dipivefrin hydrochloride, dipivalyloxy catecholamine derivatives, and dipivalyl prodrugs, or the enantiomers, diastereoisomers, racemates and the salts of such compounds with pharmaceutically acceptable counterions, or any combinations thereof.

In some embodiments, the anti-anaphylactic and/or anti-anaphylactoid agent can be an alpha 1 adrenergic receptor agonist, for example Noradrenaline, Phenylephrine, Methoxamine, Cirazoline, Xylometazoline, Midodrine, or Metaraminol. In some embodiments, the anti-anaphylactic and/or anti-anaphylactoid agent can be an alpha 2 adrenergic receptor agonist, for example Dexmedetomidine, Medetomidine, Romifidine, Clonidine, Brimonidine, Detomidine, Lofexidine, Xylazine, Tizanidine, Guanfacine, or Amitraz. In some embodiments, the anti-anaphylactic and/or anti-anaphylactoid agent can be a beta 1 adrenergic receptor agonist, for example Dobutamine, Isoprenaline, or Noradrenaline. In some embodiments, the anti-anaphylactic and/or anti-anaphylactoid agent can be a beta 2 adrenergic receptor agonist, for example Salbutamol (Albuterol in USA), Bitolterol mesylate, Formoterol, Isoprenaline, Levalbuterol, Metaproterenol, Salmeterol, Terbutaline, or Ritodrine. In some embodiments, the anti-anaphylactic and/or anti-anaphylactoid agent can be a beta 3 adrenergic receptor agonist, for example Mirabegron, L-796568, Amibegron, or Solabegron. In some embodiments, the anti-anaphylactic and/or anti-anaphylactoid agent can be glucagon, H1 receptor blockade compound, H2 receptor blockade compound, benadryl, ranitidine, prednisone, or any combination thereof, which can be used alone or in conjunction with epinephrine. The compositions herein can comprise one or more of the above anti-anaphylactic and/or anti-anaphylactoid agents, for example 2, 3, 4, 5, 6, 7, 8, 9, or 10 of them. In some embodiments, the compositions herein do not comprise a beta-blocker compound. In some embodiments, the compositions herein do not comprise cocaine, or a derivative thereof. For example, the anti-anaphylactic or anti-anaphylactoid agent does not comprise cocaine or a derivative thereof.

In some embodiments, the amount of an anti-anaphylactic and/or anti-anaphylactoid agent (e.g., epinephrine) present in the compositions and/or dosage units herein can be about 0.25% to about 50% w/w of the weight of the composition, for example about 0.25%, about 0.5%, about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, about 10%, about 11%, about 12%, about 13%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, or about 50%. In some embodiments, the anti-anaphylactic or anti-anaphylactoid agent is about 4%, about 7.5%, or about 15% w/w of the weight of the composition.

### Vasoactive Agent

The methods, kits, compositions and systems disclosed herein may comprise a vasoactive agent. In some embodiments, the vasoactive agent can be epinephrine or a pharmaceutically acceptable salt thereof. For example, the vasoactive agent can be epinephrine hydrochloride, epinephrine free base, epinephrine maleate, epinephrine bitartrate, epinephrine methyl ester or hydrochloride, glycosyl epinephrine derivatives, dipavalyl epinephrine derivatives including dipivefrin hydrochloride, dipivalyloxy catecholamine derivatives, and dipivalyl prodrugs, or the enantiomers, diastereoisomers, racemates and the salts of such compounds with pharmaceutically acceptable counterions, or any combinations thereof. In some embodiments, the vasoactive agent can be vasopressin, and/or a pharmaceutically acceptable salt thereof. In some embodiments, the vasoactive agent can be atropine, and/or a pharmaceutically acceptable salt thereof. In some embodiments, the compositions herein do not comprise a beta-blocker compound. In some embodiments, the compositions herein do not comprise cocaine, or a derivative thereof. For example, the vasoactive agent does not comprise cocaine or a derivative thereof.

In some embodiments, the amount of a vasoactive agent (e.g., epinephrine) present in the compositions and/or dosage units herein can be about 0.25% to about 50% w/w of the weight of the composition, for example about 0.25%, about 0.5%, about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, about 10%, about 11%, about 12%, about 13%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, or about 50%. In some embodiments, the vasoactive agent is about 4%, about 7.5%, or about 15% w/w of the weight of the composition.

### Carrier

The methods, kits, compositions and systems disclosed herein may comprise a pharmaceutically acceptable carrier. In one embodiment, the pharmaceutical acceptable carriers may comprise a first cellulose. In one embodiment, the first cellulose is a crystalline cellulose. In one embodiment, the first cellulose is a microcrystalline cellulose. The first cellulose may have an average particle diameter of about 150, 140, 130, 120, 110, 100, 90, 80, 70, 60, 50, 40, 30, 20, 10 μm or less. For example, the first cellulose may have an average particle diameter of about 100 μm or less. The first cellulose may have an average particle diameter of about 30 μm or less. The first cellulose may have an average particle diameter of about: 90 to 100 μm, 80 to 90 μm, 70 to 80 μm, 60 to 70 μm, 50 to 60 μm, 40 to 50 μm, 30 to 40 μm, 20 to 30 μm, or 10 to 20 μm.

In one embodiment, the pharmaceutical acceptable carriers may further comprise a second cellulose. In one embodiment, the pharmaceutical acceptable carriers may further comprise a starch. In one embodiment, the pharmaceutical acceptable carriers may further comprise a second cellulose and starch. In one embodiment, the pharmaceutical acceptable carriers may comprise a second cellulose. In one embodiment, the second cellulose is a crystalline cellulose. In one embodiment, the second cellulose is a microcrystalline cellulose. The second cellulose and/or starch can have an average particle diameter of about 30 to about 100 μm, for example about: 30-40 μm, 30-50 μm, 30-60 μm, 30-70 μm, 30-80 μm, or 30-90 μm.

The methods, kits, compositions and systems disclosed herein may comprise an anti-caking agent. In some embodiments, anti-caking agent is tribasic calcium phosphate. The tribasic calcium phosphate may have an average particle diameter of about 150, 140, 130, 120, 110, 100, 90, 80, 70, 60, 50, 40, 30, 20, 10 μm or less. For example, the tribasic calcium phosphate may have an average particle diameter of about 100 μm or less. The tribasic calcium phosphate may have an average particle diameter of about 30 μm or less. The tribasic calcium phosphate may have an average particle diameter of about: 90 to 100 μm, 80 to 90 μm, 70 to 80 μm, 60 to 70 μm, 50 to 60 μm, 40 to 50 μm, 30 to 40 μm, 20 to 30 μm, or 10 to 20 μm.

In some embodiments, the methods, kits, compositions and systems disclosed herein may comprise one or more active agents at a weight ratio of about 0.0001 to about 1.2 to the weight of a pharmaceutically acceptable carrier herein, wherein the one or more active agents are in a free form without being converted to the salt form, when the weight of the pharmaceutically acceptable carrier herein can be taken as 1. In some embodiments, the first crystalline cellulose can be present from 60 to 94.9% w/w, for example about: 60 to 70, 70 to 80, 80 to 90, or 90 to 94.9% w/w, based on the weight of the carrier. In some embodiments, the second crystalline cellulose or starch can be present from about 5 to 30% w/w, for example about: 5 to 10, 10 to 15, 15 to 20, 20 to 25, or 25 to 30% w/w, based on the weight of the carrier. In some embodiments, the carrier can further comprise tribasic calcium phosphate. In some embodiments, the tribasic calcium phosphate can be present from 0.5 to 5% w/w, for example about: 0.5 to 1, 1 to 2, 2 to 3, 3 to 4, or 4 to 5% w/w, based on the weight of the carrier. In some embodiments, the composition herein can further comprise a pH adjustor, a preservative, a stabilizer, a flavor, an absorbefacient, and/or a substance that captures a divalent calcium ion. In one aspect, provided herein is a powdery carrier formulation for nasal administration wherein the angle of repose of the carrier, formulation, and/or unit dose can be about 35° to about 55°. In some embodiments, the angle of repose of the carrier, formulation, and/or unit dose can be about: 40° to 53°, 50° to 55°, 45° to 50°, or 35° to 40°, or about: 35, 36, 37, 38, 39, 40, 41, 42, 43, 44, 45, 46, 47, 48, 49, 50, 51, 52, 53, 54, or 55 degrees.

In one aspect, a pharmaceutically acceptable carrier herein can comprise: i) crystalline cellulose (A), which can be a first crystalline cellulose with an untapped bulk density of about 0.13 to about 0.29 g/cm³, a specific surface area of about 1.3 m²/g or more, an average particle diameter of about 30 μm or less, and/or an angle of repose of about 55° or more; ii) tribasic calcium phosphate (B); and iii) crystalline cellulose (C), which can be a second crystalline cellulose with an untapped bulk density of about 0.26 to about 0.48 g/cm³, a specific surface area of about 1.3 m²/g or less, an angle of repose of about 50° or less, and/or an average particle diameter of about 150 μm or less, or starch (D) with an untapped bulk density of about 0.35 to about 0.65 g/cm³, a specific surface area of about 1.3 m²/g or less, an angle of repose of about 55° or less, and/or an average particle diameter of about 150 μm or less. In some embodiments, a pharmaceutically acceptable carrier herein can comprise about 0.1 to about 10% w/w tribasic calcium phosphate (B), about 5.0 to about 30% w/w second crystalline cellulose (C) and/or starch (D), and the remainder can be the first crystalline cellulose (A).

### Vasodilator

The methods, kits, compositions and systems disclosed herein may comprise a vasoactive agent (e.g., epinephrine) and/or an anti-anaphylactic and/or anti-anaphylactoid agent (e.g., epinephrine), that can avoid local tissue atrophy, e.g., observed often in epinephrine injections. A vasodilator can be added to the formulations and/or unit doses at subclinical concentrations to reduce the nasal vascular vasoconstriction caused by epinephrine and can allow faster flux across the mucosal membrane, and can be designed to have the minimal systemic exposure and to be acting mainly topically. This can further enable use of lower loading doses of the vasoactive agent and/or the anti-anaphylactic and/or anti-anaphylactoid agent (e.g., epinephrine), thereby reducing dose-related adverse side effects.

Accordingly, the vasodilators in the formulations herein can be older vasodilators (e.g., hydralazine, isosorbide mononitrate, isosorbide dinitrate), ACE inhibitors (e.g., Benazepril (Lotensin), Captopril (Capoten), Enalopril (Vasotec), Fosinopril (Monopril), Lisinopril (Prinivil, Zestril), Minoxidil (Loniten), Meoexipril (Univasc), Perindopril (Aceon), Quinapril (Accupril), Ramipril (Altace), Trandolaptril (Mavik)), and Angiotension II receptor agonists (A2 inhibitors) (e.g., Losartan, Candesatran, Valsartan, Irbesartan, Telmisartan, Eprosartan, Olmesartan, Azilsartan), and others including papaverine hydrochloride or phentolamine mesylate, selected from: cocaine; ethyl nitrate; nitroglycerine; diltiazem; urapidil; nicorandil; sodium nitroprusside; glyceryl trinitrate-verapamil; phenoxybenzamine; dopexamine; chloropromazine; propiverine hydrochloride; and the enantiomers, diastereoisomers, racemates, the salts of such compounds with pharmaceutically acceptable acids and bases, and any combination thereof. In one embodiment, a vasodilator in the dry powder compositions and/or dosage units herein can be phentolamine.

In some embodiments, the amount of phentolamine present in the compositions and/or dosage units can be about 0.005% to 50% w/w of the weight of the composition, for example about 0.005%, about 0.01%, about 0.05%, about 0.1%, about 0.15%, about 0.2%, about 0.25%, about 0.5%, about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, about 10%, about 11%, about 12%, about 13%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, or about 50%. In some embodiments, the amount of phentolamine is about 2.5% w/w of the weight of the composition.

### Enabling Agent and Other Ingredients

The methods, kits, compositions and systems disclosed herein may further comprising one or more enabling agents, including a reversible Catechol-O-Methyl Transferase (COMT) inhibitor, which reduces the action of natural COMT enzymes that degrade epinephrine on the nasal mucosa. Consequently, this can allow for use of lower doses of a vasoactive agent (e.g., epinephrine), and/or an anti-anaphylactic and/or anti-anaphylactoid agent (e.g., epinephrine) for administration of the formulations herein, further reducing local tissue irritation, and other adverse side effects, that are dose-dependent. Reducing the loading dose of an anti-anaphylactic and/or anti-anaphylactoid agent (e.g., epinephrine) also make the solubility limit of aqueous delivery or the mg weight burden of powder delivery, more practical. Inhibiting the action of naturally present COMT enzymes can be an approach used in the treatment of Parkinson's disease. Parkinson patients can metabolically degrade the Parkinson's drug, L-Dopa, via COMT. As a counter measure, the FDA approved use of inhibitors of oral COMT for Parkinson's treatment. Herein, drugs in this class can be used in a novel way, to reduce the degradation of a vasoactive agent and/or an anti-anaphylactic and/or anti-anaphylactoid agent (e.g., epinephrine) by COMT and potentiate and/or enhance the potency of the anti-anaphylactic and/or anti-anaphylactoid agent (e.g., epinephrine). Accordingly, a reversible COMT inhibitor(s) can be included at subclinical concentrations, and can be designed to have the minimal systemic exposure and to be acting mainly topically. The COMT inhibitors can include nitrocatechols entacapone or tolcapone, Comtan (entacapone), Stalevo (entacapone plus carbidopa and levadopa) and/or Tasmar (or the enantiomers, diastereoisomers, racemates, and salts of such compounds with pharmaceutically acceptable acids and bases).

Enabling agents useful herein can also include potentiators that improve and/or enhance the pharmacological action of an anti-anaphylactic and/or anti-anaphylactoid agent (e.g., epinephrine). Such potentiators can include guanethidine, NAC (N-acetylcysteine); isoproterenol; norepinephrine; hydrocortisone; flavonoids (vitamin-P like compounds); local anesthetics; vasopressin; cocaine; methylphenidate; tripelennamine; bufozenine; harmine; mescaline; LSD (lysergic acid diethylamide); methergine; ganglionic blockers; antihistamines; amphetamines, the enantiomers, diastereoisomers, racemates, and salts of such compounds with pharmaceutically acceptable counterions, and any combination thereof. Other agents that potentiate anti-anaphylactic and/or anti-anaphylactoid agent (e.g., epinephrine) responses by inhibiting its degradation by COMT can include tropolone, desmethyl papaverine and pyrogallol. Certain amino acids, including histidine in the presence of tissue cupric ions, can also potentiate action of a vasoactive agent and/or an anti-anaphylactic and/or anti-anaphylactoid agent (e.g., epinephrine). Further, compounds for example flavinoids (Vitamin P-like compounds), local anesthetic agents, vasopressin, cocaine, methylphenidate (Concerta®), tripelennamine, bufotenine, harmine, mescaline, LSD, methergine, ganglionic blockers, antihistamines (norepinephrine), and amphetamines can also be used to potentiate a vasoactive agent and/or an anti-anaphylactic and/or anti-anaphylactoid agent (e.g., epinephrine).

A permeation enhancer may include one or mixture of substances which when formulated with a vasoactive agent and/or an anti-anaphylactic or anti-anaphylactoid agent (e.g., epinephrine) can have the effect of increasing the fraction of the vasoactive agent and/or the anti-anaphylactic or anti-anaphylactoid agent (e.g., epinephrine) applied to a nasal mucosal surface that traverses a mucosal membrane and enters bloodstream, e.g., increases bioavailability. The addition of a permeation enhancer to a formulation designed for intranasal administration can increase the fraction of a vasoactive agent and/or an anti-anaphylactic and/or anti-anaphylactoid agent (e.g., epinephrine) that reaches the circulation by at least about 25%, at least about 50%, or at least about 100%. The formulations and/or dosage units herein can comprise nasal permeation and/or nasal absorption enhancers for example bile salts, alkyl glycosides, polymers, tight junction modulating peptides as described in the PCT publication No. WO2007014391 A2, lipids, surfactants, cyclodextrin, chelators, and any combination thereof. Cyclodextrins can have various functions in the intranasal formulation, including taste masking, drug solubilization, and drug stabilization. Cyclodextrins can also have unexpected synergistic effects when combined with certain permeation enhancers. Examples of pharmaceutically acceptable cyclodextrins include alpha-cyclodextrin, beta-cyclodextrin, gamma-cyclodextrin, hydroxypropyl-beta-cyclodextrin, and sulfobutylether beta-cyclodextrin. In certain embodiments, ‘Hsieh enhancers’ described in U.S. Pat. Nos. 5,023,252 and 5,731,303, cyclic lactones, cyclic diesters, and/or cyclic ketones described in the PCT publication No. WO2011153400 A2 can be added as permeation enhancers.

Exemplary lipids that can be included in the formulations and/or dosage units herein can include, but are not limited to, 1,2-Dioleoyl-sn-Glycero-3 Ethylphosphocholine, 1,2-di-O-phytanyl-glycero-3-phosphocholine, 1-O-hexadecyl-2-acetoyl-sn-glycerol, 1-O-octadecyl-2-O-methyl-glycerol-3-phosphocholine, 16: 0-09:0(ALDO)PC, 16:0-09:0(COOH)PC, 3-beta-hydroxy-5alpha-cholest-8(14)-en-15-one, C10 sucrose, C12 maltose, C12 sucrose, C14 maltose, C16-09:0, C6 glucose, C6 maltose, C7 glucose, C8 glucose, Cardiolipin (sodium salt), Ceramide (brain porcine), Ceramide C10:0, Ceramide C12:0, Ceramide C14:0, Ceramide C16:0, Ceramide C17:0, Ceramide C18:0, Ceramide C18:1, Ceramide C20:0, Ceramide C24:0, Ceramide C24:1, Ceramide C2:0, Ceramide C4:0, Ceramide C6:0, Ceramide C8:0, Cerebroside (brain porcine), Cerebroside Sulfatide (porcine), Dimethylsphingosine, Egg Ceramide, Galactosyl sphingosine, Glucosyl-sphingosine, Lactosyl(β) Sphingosine, Lyso-PAF, N-acetoyl ceramide−1-phosphate, N-octanoyl ceramide−1-phosphate, PGPC1, POVPC, Phosphatidylinositol (Soy), Phosphatidylinositol (bovine), Platelet-Activation Factor, Porcine brain ganglioside, Sphingomyelin (brain porcine), Sphingosine-1-phosphate, and rimethylsphingosine. Lipids in this context can be those classified as glycosylated sphingosines, alkylglucosides, oxidized lipids, and ether lipids (PAF).

A variety of commonly used and generally accepted as safe (GRAS) pharmaceutical excipients that can be included in the formulations and/or dosage units herein surprisingly can act synergistically to increase a vasoactive agent and/or an anti-anaphylactic and/or anti-anaphylactoid agent (e.g., epinephrine) permeation across the nasal mucosa. Those excipients can be demonstrated by their ability to improve the vasoactive agent and/or the anti-anaphylactic and/or anti-anaphylactoid agent (e.g., epinephrine) permeation in vitro, for example in an in vitro tissue model to human mucosa, and also in vivo, for example, in animal pharmacokinetic studies. Alternatively, we have also found that near-GRAS and non-GRAS excipients can act synergistically upon the nasal mucosa to increase transmucosal permeation of the vasoactive agent and/or the anti-anaphylactic and/or anti-anaphylactoid agent (e.g., epinephrine).

Permeation enhancers suitable for use in the formulation of drug preparations that enter the bloodstream via the GI tract, can also be adapted for use in the formulations and/or unit doses herein. These, without limitation, include those disclosed in U.S. patent publication No. 20030232078 for example ethylene-diamine tetra-acetic acid (EDTA), bile salt permeation enhancers for example those noted above and fatty acid permeation enhancers, for example sodium caprate, sodium laurate, sodium caprylate, capric acid, lauric acid, and caprylic acid, acyl carnitines, for example palmitoyl carnitine, stearoyl carnitine, myristoyl carnitine, and lauroyl carnitine, and salicylates, for example sodium salicylate, 5-methoxy salicylate, and methyl salicylate. U.S. Pat. Nos. 4,548,922 and 4,746,508 also disclose systems for delivering proteins and polypeptides by intranasal or other transmucosal routes using low toxicity permeation enhancers of the amphiphilic steroid family, e.g. fusidic acid derivatives, e.g., to promote efficient transport of the drug across the mucosal surface.

For permeation enhancement of a vasoactive agent and/or an anti-anaphylactic and/or anti-anaphylactoid agent (e.g., epinephrine), the actual effectiveness of an enhancer can be verified by using the porcine or rat model. The amount of permeation enhancer that can be included in the formulations can range, for example, from about 1 wt % to about 30 wt % based on the weight of the formulation. The precise nature and amount of enhancer will vary depending on, for example, the particular permeation enhancer or enhancer composition selected, and on the nature of other components in the formulation, for example its potency. The upper limit for enhancer concentration can be set, for example, by toxic effect to, irritation limits of the mucosal membrane, its solubility limits, or any combination thereof.

Enabling agents useful herein can also include mucosal absorption or transport enhancers, mucosal transit slowing agents and/or mucoadhesives. Because mucosal membranes provide a protective barrier against the outside environment and can be lined by epithelial cells which provide a barrier to the entry of toxins, bacteria and viruses, agents that aid or promote absorption and/or transport of therapeutic agents by getting past the protective barrier can be used in pharmaceutical compositions. Absorption agents can include surfactants, gelling microspheres and/or the bioadhesive polymer, chitosan. Examples of these systems have been reviewed by Ilium and Fisher in “Inhalation Delivery of Therapeutic Peptides and Proteins,” Adjei and Gupta (eds.) Marcel Dekker Inc, New York (1997), 135-184 and by Costantino, Ilium, Brandt, Johnson and Quay, Intranasal delivery: Physicochemical and Therapeutic Aspects, lnt J Pharm, 337, 2007, 1-24. The formulations and/or dosage units herein can comprise one or more of the afore-mentioned absorption agents or others including sodium lauryl sulfate, sodium salicylate, oleic acid, lecithin, dehydrated alcohol, Tween™, Span™, polyoxyl 40 stearate, polyoxyl ethylene 40 stearate, propylene glycol, hydroxyl fatty acid ester of polyethylene glycol, glycerol monooleate, fusieates, bile salts, octoxynol, polysorbate 20, polysorbate 80, DDPC, DPPC, a chelator for example EDTA, EGTA, citrate, and combinations thereof; and/or one selected from the group consisting of anionic, cationic and nonionic surfactants. The term “enhancer” as used herein can also encompass substances that are capable of modulating the barrier function of a cellular tight junction.

Since the typical residence time of proteins and other macromolecular species delivered can be limited at the nasal mucosa due to rapid mucociliary clearance, e.g., to about 15-30 minutes or less, in some embodiments, substances, compounds or peptides that reduce nasal mucosal transit time can be included in the intranasal compositions and/or dosage units herein. For instance, polyacrylate mucoadhesive agents can slow the rate of gastric transit thereby maximizing efficiency of both the protective effect and the time required for delivery of repair agents into the underlying tissue. The intranasal formulations and/or dosage units herein can comprise polyacrylate mucoadhesive agents as disclosed in the PCT publication No., WO2003037355A1 or similar agents or substances (synthetic or natural) or peptides, which can be compatible with administration of anti-anaphylactic and/or anti-anaphylactoid agent (e.g., epinephrine), and can slow the rate of nasal transit and maximize the absorption of the vasoactive agent and/or the anti-anaphylactic and/or anti-anaphylactoid agent (e.g., epinephrine) in the nasal mucosa.

In some embodiments, the intranasal compositions and/or dosage units herein can include modulatory agents of epithelial junction physiology, for example nitric oxide (NO) stimulators, chitosan and/or chitosan derivatives.

To promote adhesion of the active ingredients of the pharmaceutical compositions and/or dosage units herein, the methods, kits, compositions and/or dosage units herein can comprise mucoadhesive agents to the intranasal composition. As used herein, mucoadhesion can be a property whereby a natural or synthetic substance, when applied to a mucosal epithelium, adheres to or is absorbed into a subject's mucosal membrane for a period of time sufficient to quantitatively deliver an anti-anaphylactic and/or anti-anaphylactoid composition provided herein to the subject. Adhesion of mucoadhesives to a mucous membrane can occur via secondary chemical bonds, for example hydrogen bonding, and Van der Waal forces (Tabor et al., 1977 J. Colloid Interface Sci. 58:2 and Good 1977 J. Colloid Interface Sci. 59:398). Non-limiting examples of one or more mucoadhesive agents that can be added to the present compositions and/or dosage units include microcrystalline cellulose, cellulose derivatives, starch, proteins for example mucin, lactoferrin and transferrins, mucoadhesive polymers for example chitosan or carbopol, polyacrylic acid and/or derivatives for example Carbophil, Carbomer, carbopol 943, lecithin, or any combination thereof (Takeuchi et al., 2005. Adv Drug Delivery Reviews, 57:1583-1594).

The methods, kits, compositions and/or dosage units herein may comprise one or more pharmaceutically acceptable excipients, including block copolymers comprising repeating moieties, e.g., ethylene oxide moieties, anionic polysaccharides, ion exchange polymeric materials, excipients (e.g., pectin, carboxylated starch, and gellan), and any combination thereof.

Viscosity enhancing or thickening agents, especially in nasal dry powder delivery, can also have other desirable actions on the nasal mucosa for increasing API transport or absorption, dissolution rate, or residency time, and can include e.g., poly (vinyl alcohol) (PVA), poly (ethylene glycol) (PEG), propylene glycol, and polysaccharides for example soluble starch, various cellulose forms both crystalline and amorphous, methylcellulose, hydroxylpropyl cellulose carboxymethylcellulose, chitosan, and any combination thereof.

Because epinephrine has been reported to have an unpleasant taste when inhaled (Simons F. E. R. et al., 2000 Pediatrics 106(5): 1040-44) addition of tastemasking agents to the compositions and/or dosage units could be valuable. The formulations and/or dosage units herein can use a variety of tastemasking agents, including cyclodextran cages to tastemask. Other agents for taste masking include, but are not limited to citric acid (for example up to 20% in a marketed nasal solution), sorbitol (for example up to 2.86% in a marketed metered nasal spray), glycerin (for example up to 2.5% in a marketed nasal solution), dextrose (for example 5% in a marketed metered nasal spray), and phenethyl alcohol (for example up to 0.25% in a marketed metered nasal spray, also could serve as a preservative for multi-use). The following agents approved in buccal/oral/dental compositions can also be added in the formulations and/or dosage units herein: acacia syrup, anethole, anise oil, benzaldehyde, butterscotch, cardamom, cherry (and varieties thereof), cinnamon, cocoa, coriander, ethyl acetate, ethyl vanillin, ginger, glucose, lavender, lemon, maltodextrin, mannitol, methyl salicylate, nutmeg, orange, peppermint, raspberry, saccharin, spearmint, sucrose, sucralose, tolu, vanilla, varieties, and any combination thereof.

Sulfite-free, non-toxic preservatives for a vasoactive agent, and/or an anti-anaphylactic and/or anti-anaphylactoid agent (e.g., epinephrine) (or other oxidizable drugs) that are non-allergenic and non bronchospasmodic can include thiols, glutathione, glutathione reductase, glutathione peroxidase, hydroquinone, amikasin sulfate, apomorphine hydrochloride, metaraminol, levobunonol, levobunonol hydrochloride, acamprosate calcium, fenoldopam, hydrocortisone/neomycin sulfate/polymyxin B, dexamethasone sodium phosphate, hydromorphone, dobutamine, etidicaine/epinephrine bitartrate, gentamycin, tinzaparin, isoproternerol, ketoconazole, sodium sulfacetamide, norepinephrine, bupivacaine/epinephrine bitartrate, morphine, tobramycin, rotigotine, orphenadrine, procaine, nalbuphine, oxytetracycline, nortriptyline, perphenazine, promethazine hydrochloride, prednisolone acetate, propofol, mesalamine, trimethoprim/sulfamethoxazole, carisoprodol/aspirin/codeine, streptomycin, mafenide acetate, tetracycline hydrochloride, pentazocine lactate, chlorpromazine, triethylperazine maleate, fluorinolone acetonide/hydroquinone/tretinoin, acetaminophen/codeine, doxycline calcium, lidocaine/epinephrine, and any combination thereof.

Stabilizers can be themselves bronchodilators and/or allergenic at least in sensitive patients, especially asthmatics. Such preservatives include: sodium metabisulfite (for example 0.5 g in EpiPen™); chlorobutanol and sodium metabisulfite (Auvi-Q™ injection, at unspecified concentrations); and for example 34% dehydrated alcohol with Vitamin C (in Primatene Mist Inhalation Aerosol). The bisulfites, chlorobutanol and alcohol inactive ingredients have all been extensively documented as being contraindicated for bronchospasm, and by implication for anaphylaxis, anaphylactoid reactions, and/or cardiac arrest. The formulations and/or dosage units herein can contain no bronchospasmodic preservatives, only Vitamin C as a possible stabilizer and being sealed in a darkened vial under inert nitrogen. Vitamin C can be harmless in relation to lung function, bronchospasm and asthma. Vitamin C is recognized as the major antioxidant in airway surface liquid of the lung, where it is likely protective against toxic oxidants and can have this effect on the nasal mucosa.

Antihistamines can not only act to inhibit COMT but also can prevent explosive mast cell degranulation activity in response to allergens. The antihistamines can include those suitable for nasal application as disclosed in published US application 20100055152 A1, non-sedating antihistamines disclosed in issued U.S. Pat. No. 8,263,581, azelastine, hydroxyzine, desloratadine, emadastine, levocabastine, carbinoxamine, levocetrizine, fexofenadine, diphenhydramine, brompheniramine, clemastine, chlorpheniramine, and any combination thereof.

Humectants can include sorbitol, glycerol, mineral oil, vegetable oil, and any combinations thereof.

Osmotic adjusting agents, which can be used, include, but are not limited to, sodium chloride, potassium chloride, zinc chloride, calcium chloride, and mixtures thereof. Other osmotic adjusting agents can also include, but are not limited to, mannitol, glycerol, and dextrose and mixtures thereof. In an alternative embodiment, the formulations and/or unit dosages herein can comprise about 0.4 to about 1.0 weight percent ionic salt. The compositions and/or unit dosages herein can comprise about 0.9 weight percent of an osmotic adjusting agent.

The subject can be a mammal including a human, a domestic livestock, a laboratory subject, or a pet animal, and including any of those in need thereof.

### Modes for Carrying Out the Invention

In one embodiment, the composition herein can be a dry powder formulation, including one selected from the group consisting of homogenous powder, heterogeneous powder, crystalline or amorphous mixtures, powder microspheres, coated powder microspheres, including micronized and nanoformulated powders, aggregates, and combinations thereof. In a related aspect, particles of a vasoactive agent, and/or an anti-anaphylactic and/or anti-anaphylactoid agent (e.g., epinephrine), vasodilator (e.g., phentolamine), pharmaceutically acceptable carrier, other enabling agents for example mucoadhesives, mucosal permeation, absorption and/or transport enhancers and mucosal transit slowing agents and/or their salts thereof, contained in the powder formulation can be substantially amorphous or crystalline or semi-crystalline and/or semi-amorphous and/or dispersed in nature.

For easy dispensability of the powder formulation and/or unit dosages herein, the intranasal compositions can further contain additional agents including lubricants for example magnesium stearate, and/or fluidizing agents for example talc and/or silicon dioxide. The lubricants and fluidizing agents can adhere onto a powder surface and increase the space among the particles and consequently, reduce the friction and adhesion among powder particles and/or produce the dispensability improving effect.

The powder composition in some embodiments can be colorless. In other embodiments, a non-allergenic colorant can be added to the compositions and/or unit dosages herein to aid in the visualization of the dose to be dispensed from the delivery device.

In some embodiments, the shape and size of the powder particles individually or collectively can be either uniform or diverse and can be designed to have no negative influence on their absorption in the nasal mucosa. In some embodiments, the median particle diameter of particles can be individually or collectively up to 100 μm, from about 50 μm to about 100 μm, or from about 20 μm to about 50 μm. In some embodiments, the particles can be bimodal and/or vary in size to improve delivery and/or absorption of active ingredients in the dry powder compositions and/or unit dosages herein. In some embodiments, the active ingredients of the formulations and/or dosage units can exist as nanoparticles. Less than 10% of particles can be for example less than 10 microns in diameter.

In some embodiments, methods can be employed to make a suitable powder formulation and/or a unit dosage herein, including for example mixing powdery drugs with carriers and enabling agents by using for example a mortar, a mixer, and/or a stirrer. In some embodiments, preparation of a solution of active ingredients and excipients, can be followed by precipitation, filtration, and/or pulverization, and/or followed by removal of solvent by freeze-drying, and/or followed by pulverization or nanosizing of the powder to the desired particle size. Sieving can obtain particles with a size of less than for example 100 μm in diameter, from about 50 μm to about 100 μm, or about 20 μm to about 50 μm in diameter. In some embodiments, nasal powder compositions made by mixing a vasoactive agent, and/or an anti-anaphylactic and/or anti-anaphylactoid agent (e.g., epinephrine), enabling agent(s), including mucosal permeation enhancers, agents that increase nasal residency time, and acceptable excipients, can each possess the desired particle size.

In another embodiment, a process for making a sterile nasal composition without preservatives can comprise one or more of the following: (1) adding at least a therapeutically effective pediatric or, alternatively adult, amount of a vasoactive agent, and/or an anti-anaphylactic or anti-anaphylactoid agent (e.g., epinephrine) and enabling drug(s) and other agent(s) in a vehicle, for example water; and (2) placing the resulting mixture in a container, and sterilizing the mixture, for example by steam sterilization. Each mixture can be filled into a vial, and then packaged under nitrogen gas, sealed, stored and/or used directly. Here, the resulting mixture can be stable, and after sterilization, it can be dispersed, if necessary, into multiple mixtures each containing a unit dose of a therapeutically effective amount of an anti-anaphylactic and/or anti-anaphylactoid agent (e.g., epinephrine) and enabling drugs suitable for adults or children.

In another embodiment, a process for making a non-sterile nasal dry powder composition with preservatives can comprise one or more of the following: (1) adding at least a therapeutically effective pediatric or, alternatively adult, amount of an anti-anaphylactic or anti-anaphylactoid agent (e.g., epinephrine) with a carrier herein; and (2) adding stabilizers for example BAC, and either sodium metabisulfite or ascorbic acids, in sufficient concentrations to achieve a 12-24 month shelf life or more. Each mixture can be filled into a vial, and then packaged under nitrogen gas, sealed, stored and/or used directly. Here, the resulting mixture can be stable, and can be dispersed, if necessary, into multiple mixtures each containing a unit dose of a therapeutically effective amount of an anti-anaphylactic or anti-anaphylactoid agent (e.g., epinephrine) suitable for adults or children.

In some embodiments, osmotic adjusting agents can be used herein include, but are not limited to, sodium chloride, potassium chloride, zinc chloride, calcium chloride, and mixtures thereof. Other osmotic adjusting agents can also include, but are not limited to, mannitol, glycerol, and dextrose and mixtures thereof. In an alternative embodiment, the formulations and/or unit doses herein can comprise about 0.4 to about 1.0 weight percent ionic salt based on the weight of the formulations and/or unit doses. The formulations and/or unit doses herein can comprise about 0.9 weight percent of an osmotic adjusting agent based on the weight of the formulations and/or unit doses.

In some embodiments, the intranasal formulations and/or unit doses herein can comprise a variety of tastemasking agents, including cyclodextran cages (Simons F E et al., 2000 Pediatrics 106(5): 1040-44).

The powder compositions herein can be presented in a sterile unit dosage form (for example, in capsules, cartridges, or blister packs) from which the powder can be administered with the aid of a dry powder dispenser or other nasal delivery method.

One or more pharmaceutical dose of the dry powder composition herein can be administered using a simple hand or finger operated spray device, nasal insufflator, a jet-spray, or any other device.

In certain other embodiments, a vasoactive agent, and/or anti-anaphylactic or anti-anaphylactoid agent (e.g., epinephrine) can be nanoformulated and can be dosed as a reconstituted powder prior to administration, or as a liquid suspension. ‘Nanoformulation’ herein can utilize a readily available and simple fluid bed spray drying method in a manner that produces uniform and stable for example about 40-90 nm (e.g., 40, 50, 60, 70, 80, or 90 nm) drug particles of pure API without external excipients. This can be important since some methods of manufacturing nanopharmaceuticals require the addition of external nanostructures to the API, which raises potential safety concerns.

One advantage of nanoformulation can be the tiny size of the a vasoactive agent, and/or the anti-anaphylactic and/or anti-anaphylactoid agent (e.g., epinephrine) particles and the vast increase in surface area compared to non-nanoformulated vasoactive agent, and/or anti-anaphylactic and/or anti-anaphylactoid agent (e.g., epinephrine). Other advantages of nanoformulation include (i) higher water solubility of drugs that are poorly soluble in water, (ii) drug solubility limits can be reduced, (iii) absence of any food effects, which can limit patient compliance if a drug needs to be taken with food or without food, (iv) reduced hepatic first pass metabolism by unclear mechanisms and/or, and (v) increased rate of membrane transportation.

### Pharmaceutical Product and Doses

In one aspect, provided herein is a pharmaceutical product, comprising a pharmaceutical apparatus for intranasally administering a pharmaceutical dose, and/or a pharmaceutical composition disclosed herein. The apparatus can comprise a reservoir and means for expelling the pharmaceutical dose in the form of a dry powder or aqueous spray, wherein a quantity of the pharmaceutical composition can be contained within the reservoir. In an embodiment, the apparatus can comprise a pump spray device in which the means for expelling a dose can comprise a metering pump or precise expulsion of the correct dose in a single-use device. In an alternative embodiment, the apparatus can comprise a pressurized spray device, in which the means for expelling a dose can comprise a metering valve and the pharmaceutical composition can further comprise a conventional propellant. Suitable pressurized spray devices can include those disclosed in, the PCT publication WO92/11190, U.S. Pat. No. 4,819,834, U.S. Pat. No. 4,407,481 and the PCT publication WO97/09034, when adapted for producing a nasal spray, rather than an aerosol for inhalation, or a sublingual spray. Suitable nasal pump spray devices include the VP50™ VP70™ and VP100™ models available from Valois S. A. in Marly Le Roi, France and the 50, 70 and 100 μl nasal pump sprays available from Pfeiffer GmbH in Radolfzell, Germany. In some embodiments, a capsule containing the pharmaceutical dose can be loaded into a simple intranasal delivery device such as Fit-Lizer™ from SNBL, Ltd. with a breath-monitoring device. Other models and sizes can be employed without being limited to. A pharmaceutical dose or dose unit herein can be present within the metering chamber of the metering pump or valve.

In one aspect, as shown in Table S-1, the currently available doses offered by EpiPen™ can result in physicians being forced to decide whether to underdose (e.g., 1.3-1.7 X) or to overdose (e.g., 1.5-3.0 X) a patient based on weight, especially in children. In some embodiments, provided herein are two nasal sprays: a higher dose for adults, and a lower dose for children. However, unlike EpiPen™ that is marketed only in 2 fixed doses (0.15 mg for pediatric use and 0.3 mg for adult use), the concentration of a vasoactive agent, and/or an anti-anaphylactic or anti-anaphylactoid agent (e.g., epinephrine) (the loading dose) administered in the formulations and/or unit doses herein can importantly allow the patient or healthcare professional to choose the number of sprays to administer to achieve a suitable dose for their particular body weight (Table S-1). This can be achieved, for example by having several sprays from a multi-dose pump device or using several single use pumps. Thus the formulations herein can have at least 2 key advantages (i) attaining bioequivalent doses of intramuscularly administered anti-anaphylactic or anti-anaphylactoid agent (e.g., epinephrine) for both children and adults and/or (ii) doses depending on the body weight of the subject in contrast to under- or over-dosed subjects using EpiPen™.

For instance, as shown in Table S-1, assuming that the loading dose in the present formulation (in a Nasal Epinephrine Jr nasal product) can be bioequivalent to the IM systemic blood epinephrine levels (pediatric dose of EpiPen™ is 0.15 mg/0.3 mL or 0.05 mg/100 μL), a in one embodiment, a single spray could be 0.05 mg dose per 100 μL and up to 3 sprays would deliver the blood equivalent of an IM injection of 1.5 mg and 6 sprays (e.g., 3 sprays per nostril) would deliver the blood equivalent of an intramuscular injection of 0.3 mg in a pediatric patient weighing about 30 kg. For the adult Nasal Epinephrine product, in another embodiment, the initial dose would be bioequivalent of 0.3 mg intramuscular dosing, and if more was needed an additional 6 sprays would be available. The formulations herein can also utilize the customizable dosing (e.g., 0.01 mg/kg body weight) according to Table S-2 and Table S-3.

Naturally, these loading doses can be adjusted for the actual kinetics of nasal delivery in this composition in order to achieve the bioequivalent of the intramuscular dose. After each anaphylactic event, the disposable nasal spray device may be discarded as the nitrogen seal may be broken and the unit may no longer be sterile. This formulation and drug-device combination meets the mg/kg drug dosing flexibility that clinicians have been trying to achieve for many years. Unlike Primatene Mist, which took up to 20 inhalation doses to come close to therapeutic levels (Simons et al., 2000 Pediatrics 106(5): 1040-44), each nasal spray can be manually actuated by either the patient or caregiver, and 100-250 μL in volume or 100 mg, requiring no inspiratory effort by the patient.

TABLE S-1
Illustrative Flexible Nasal Dosing at Pediatric Weights

| | Body wt Kg | | | | | |
|---|---|---|---|---|---|---|
| | ≦5 | 10 | 15 | 20 | 25 | ≧30 |
| epinephrine | 0.05 | 0.1 | 0.15 | 0.2 | 0.25 | 0.3 |
| Dose (mgs) | | | | | | |
| EpiPen Jr dose (mgs) | 0.15 | 0.15 | 0.15 | 0.15 | 0.15 | 0.15 |
| Overdose: | 3X | 1.5X | Desired | | | |
| Underdose: | | | | 1.3X | 1.7X | 2.0X |
| Nasal Epinephrine* | | | | | | |
| Sprays total | 1 | 2 | 3 | 4 | 5 | 6 |
| Sprays per nostril | 1 | 1 | 1.5 | 2 | 2.5 | 3 |
| Dose (mgs) | 0.05 | 0.1 | 0.15 | 0.2 | 0.25 | 0.3 |

| | |
|---|---|
| Desired dosing | Desired |

*The nasal loading dose will be adjusted to give blood bioequivalent doses for IM administration.

TABLE S-2
Illustrative Customizable Dosing for Children

| Drug | Dose per Spray*** | | | | | |
|---|---|---|---|---|---|---|
| 1. PEDIATRIC: | | | | | | |
| Nasal Sprays total* | 1 | 2 | 3 | 4 | 5 | 6 |
| Pediatric Body weight | ≦5 | 10 | 15 | 20 | 25 | ≧30 |
| (Kg) | | | | | | |
| Epinephrine actual | 0.05 | 0.1 | 0.15 | 0.2 | 0.25 | 0.3 |
| dose: 0.05 mg | | | | | | |
| Desired Dose: | 0.05 | 0.1 | 0.15 | 0.2 | 0.25 | 0.3 |
| Phentolamine actual | 0.1 | 1.0 | 1.5 | 2.0 | 2.5 | 3.0 |
| dose: 0.5 mg | | | | | | |
| Desired Dose: | | | | | | |
| 0.6-30 mg/mL spray | | | | | | |
| Entacapone actual | 0.1 | 0.2 | 0.3 | 0.4 | 0.5 | 0.6 |
| dose: 0.1 mg | | | | | | |
| Desired dose under | | | | | | |
| 20 mg/spray | | | | | | |

TABLE S-3
Illustrative Customizable Dosing for Adults

2. ADULT**: Dosage is one spray, unless treatment a failure or rebound anaphylaxis and/or anaphylactoid
reactions requires a second, to a maximum of fouradditional doses total for refractory patient or rebound
anaphylaxis; the loading doses are illustrative and would be adjusted for bioequivalence to IM injection of
epinephrine

| Sprays total* | 1 | 2 | 3 | 4 |
|---|---|---|---|---|
| Adult Body weight (Kg) | ≧30 | ≧60 | ≧90 | ≧120 |
| Epinephrine actual dose: | 0.3 mg | 0.6 | 0.9 | 1.2 |
| Desired Dose: | 0.3 mg | na | na | na |
| Phentolamine actual dose: | 0.5 mg | 0.5 | 1.0 | 1.5 |
| Desired Dose: 0.1-30 mg/mL spray | | | | |
| Entacapone actual dose: | 0.1 mg | 0.1 | 0.2 | 0.3 |
| Desired dose: 0.1-20 mg/spray | | | | |

*Sprays divided between two nostrils; so for 6 sprays total, this is 3 sprays per nostril; for 3 sprays total, this is 2 sprays in one nostril, 1 spray in the other, and so forth.
For Adult dosing, there would be four total sprays of 0.3 g each spray available if the first spray were ineffective.
*** Spray volume is targeted at 100 μL each spray.

This gives target concentrations of:

| | Dose per Spray | | | | Concentration* | |
|---|---|---|---|---|---|---|
| | Pediatric | | Adult | | Pediatric | Adult |
| Epinephrine | 0.05 | g | 0.30 | g | 0.5 mg/mL | 1.0 mg/mL |
| Phentolamine | 0.5 | mg | 0.5 | mg | 50 mg/mL | 50 mg/mL |
| Entacapone | 0.1 | mg | 0.1 | mg | 10 mg/mL | 10 mg/mL |

*Assume 100 μL per dose spray

Therefore unlike the marketed intramuscular epinephrine injections that are available in only two fixed doses of epinephrine, the nasal spray dosage can be adjusted 10-fold in pediatric use and 10-fold in adult use, by administering up to for example three sprays into each nostril, suitable for giving a body-weight-adjusted dose of each drug, without the need for mixing. This means that the patient can receive for example the exact weight-matched doses, or if non-responsive, additional doses. Additionally, the present formulations and/or unit doses can be delivered as a second dose from the same device in the case of rebound anaphylaxis and/or anaphylactoid reactions. In contrast, the Auvi-Q™ autoinjector has a second needle dosing system to address rebound anaphylaxis and/or anaphylactoid reactions, which was found to be confusing to patients. Finally, by intranasal administration of present formulation the formulations herein obviate the documented problem that the increasing obesity epidemic has caused the length of the EpiPen™ autoinjector needle to be insufficient to achieve intramuscular dosing, but rather subcutaneous dosing with an ineffective Tₘₐₓ.

Herein, the loading dose of a vasoactive agent, and/or an anti-anaphylactic or anti-anaphylactoid agent (e.g., epinephrine) (e.g., amount of epinephrine administered nasally) can result in a bioequivalent dose (in terms of peripheral blood levels and systemic exposure of the vasoactive agent, and/or the anti-anaphylactic or anti-anaphylactoid agent (e.g., epinephrine)) of intramuscularly injected vasoactive agent, and/or anti-anaphylactic or anti-anaphylactoid agent (e.g., epinephrine), which, in one embodiment, is 0.15 mg in children and 0.3 mg in adults, respectively. Thus, the blood bioequivalent epinephrine target levels, and the loading doses of vasodilator and COMT inhibitor can be at a ratio of (an anti-anaphylactic or anti-anaphylactoid, e.g., epinephrine): (a vasodilator, e.g., phentolamine): (a COMT inhibitor, e.g., entacapone) in the compositions is within the range of (0.15 mg: 0.5 mg: 0.1 mg) for pediatric dosing to (0.3 mg: 0.5 mg: 0.1 mg) for adult dosing. This can be increased six fold proportionately to give the estimated maximal blood doses (e.g., the blood equivalent of intramuscularly injected EpiPen™), respectively.

Upon intranasal administration of the loading dose in a powdery or aqueous form, the increase in levels of an anti-anaphylactic or anti-anaphylactoid agent (e.g., epinephrine) can be at least the same or equivalent to intramuscularly injected vasoactive agent, and/or anti-anaphylactic or anti-anaphylactoid agent (e.g., epinephrine). In another embodiment, the increase in levels of a vasoactive agent, and/or an anti-anaphylactic and/or anti-anaphylactoid agent (e.g., epinephrine) in the blood can be 2-fold more than the baseline (e.g., levels prior to epinephrine administration). In related embodiments, intranasal administration of the present composition can elicit 2-fold to 15-fold increase in the vasoactive agent, and/or the anti-anaphylactic and/or anti-anaphylactoid agent (e.g., epinephrine) levels in blood within 30 minutes of the administration.

In some embodiments, the dry powder composition when administered to a patient, produces a maximal blood concentration (Cmax) of the anti-anaphylactic or anti-anaphylactoid agent at least 2-fold, 3-fold, 4-fold, 5-fold, 6-fold, 7-fold, 8-fold, 9-fold, 10-fold, 11-fold, 12-fold, 13-fold, 14-fold, or 15-fold more than the baseline level of the anti-anaphylactic and/or anti-anaphylactoid agent in the patient. In some embodiments, the dry powder composition when administered to a patient, reaches a maximal blood concentration (Tmax) of the anti-anaphylactic or anti-anaphylactoid agent in less than about 30 minutes, 20 minutes, 10 minutes, 5 minutes, 4 minutes, 3 minutes, 2 minutes or 1 minute after administration. In some embodiments, the increase in anti-anaphylactic and/or anti-anaphylactoid agent (e.g., epinephrine) levels is observed and sustained in the blood for at least 90, 80, 70, 60, 50, 40, 30, 20, 15, or 10 minutes after administration.

In some embodiments, the dry powder composition when administered to a patient, produces a maximal blood concentration (Cmax) of the vasoactive agent at least 2-fold, 3-fold, 4-fold, 5-fold, 6-fold, 7-fold, 8-fold, 9-fold, 10-fold, 11-fold, 12-fold, 13-fold, 14-fold, or 15-fold more than the baseline level of the vasoactive agent in the patient. In some embodiments, the dry powder composition when administered to a patient, reaches a maximal blood concentration (Tmax) of the vasoactive agent in less than about 30 minutes, 20 minutes, 10 minutes, 5 minutes, 4 minutes, 3 minutes, 2 minutes or 1 minute after administration. In some embodiments, the increase in the vasoactive agent (e.g., epinephrine) levels is observed and sustained in the blood for at least 90, 80, 70, 60, 50, 40, 30, 20, 15, or 10 minutes after administration.

### Cardiac Arrest

Cardiac arrest or out of hospital cardiac arrest (OHCA) or conditions that require implementation of ACLS or AED is defined as cessation of cardiac mechanical activity that occurs outside of the hospital setting, in emergency care and is confirmed by the absence of signs of circulation. Cardiac arrest may be secondary to ventricular fibrillation, ventricular tachycardia, pulseless electrical activity, or asystole. Severe bradycardia may also require ACLS. OHCA can occur from non-cardiac causes (i.e., trauma, drowning, overdose, asphyxia, electrocution, primary respiratory arrests and other non-cardiac etiologies) or have cardiac etiologies. The intranasal dry powder composition of the present invention can be used to treat cardiac arrest arising out of cardiac or non-cardiac reasons and can be used as first line treatment or secondary treatment or as a treatment option in an AED system. Another consideration for the use of vasoactive agents may be in the setting of severe hypotension (hypotensive shock) secondary to a number of causes including, trauma, hypovolemia, bradycardia, and septic shock. The intranasal dry powder composition of the present invention can be used to treat hypotensive shock arising out of cardiac or non-cardiac reasons and can be used as first line treatment or secondary treatment.

In some embodiments herein, minimal cardiac activity can mean that the ejection fraction of a patient's or subject's left and right ventricles is about 0%, about 1% about 2%, about 3%, about 4%, about 5%, about 6%, about 7%, about 8%, about 9%, about 10%, about 20%, about 30%, about 40%, about 50%, about 0% to about 50%, about 0% to about 40%, about 0% to about 30%, about 0% to about 20%, about 0% to about 10%, or about 0% to about 5%. In some embodiments, ejection fraction can be measured by echocardiography, in which the volumes of the heart's chambers (e.g., left and right ventricles) are measured during the cardiac cycle. In some embodiments, the volume of blood within a ventricle immediately before a contraction is known as the end-diastolic volume (EDV) and the volume of blood left in a ventricle at the end of contraction is end-systolic volume (ESV) and the difference between EDV and ESV represents a ratio between the ventricles full and emptied. This ratio allows many variables such as stroke volume (SV). In some embodiments SV describes a volumetric measurement of blood ejected from the right and left ventricles with each heartbeat and ejection fraction can be the fraction of the end-diastolic volume that is ejected with each beat; that is, it is stroke volume (SV) divided by end-diastolic volume (EDV):

Ef(%)=SV/EDV(100%)

Where the stroke volume is given by:

SV=EDV−ESV

### Pharmaceutical Kit

The methods, kits, compositions, and/or systems herein may comprise a kit comprising: (a) a dose of an intranasal dry powder composition disclosed herein; (b) instructions reciting when the dry powder composition in (a) is to be administered to a subject. In some embodiments, the kit may further comprise an intranasal delivery apparatus for dispensing the dry powder composition. In some embodiments, the apparatus delivers a therapeutically acceptable amount of the dry powder composition. In certain embodiments, the dry powder composition is delivered intranasally. In some embodiments, the apparatus further comprises a reservoir that holds the dry powder composition. In some embodiments, the reservoir is disposable. In some embodiments, the reservoir is reusable or recyclable.

In some embodiments, the kit disclosed herein is a multi-unit package. For example, the package may comprise multiple reservoirs, wherein each reservoir contains a single dose of the dry powder composition. In another embodiment, the package may comprise one apparatus and multiple reservoirs, wherein each reservoir contains a single dose of the dry powder composition. In another embodiment, the package may comprise multiple apparatuses, wherein each apparatus contains one reservoir which contains a single dose of the dry powder composition. In another embodiment, the package may allow for easy and quick visual verification of units used. For example, the package can be labeled. Further, the package, in some embodiments, can be color labeled.

The kit disclosed herein may further comprise an automated external defibrillator (AED) system. In some embodiments, the instructions in the kit may further recite how to operate the automated external defibrillator (AED) system. In some embodiments, the instructions in the kit may be pre-loaded on the automated external defibrillator (AED) system. In some embodiments, the automated external defibrillator (AED) system may contain a self-contained power source. For example, the automated external defibrillator (AED) system may contain a battery. In a certain embodiment, the automated external defibrillator (AED) system may contain a rechargeable battery.

### Methods of Using the Dry Powder Composition

In a yet further aspect, The methods disclosed herein may comprise the use of a vasoactive agent, and/or an anti-anaphylactic or anti-anaphylactoid agent (e.g., epinephrine) in combination with one or more enabling agents, mucosal absorption agents, and/or mucosal transit agents, for the preparation of a pharmaceutical composition, dose or product herein. This can be used, e.g., for increasing coronary perfusion pressure during cardiac arrest, improving chances for ROSC, increasing mean arterial pressure during hypotensive shock, and/or treating anaphylaxis, anaphylactoid reactions, bronchospasm, and/or during CPR. One unit dose can be for example about 0.05 mg to about 10 mg of a vasoactive agent, and/or an anti-anaphylactic or anti-anaphylactoid agent (e.g., epinephrine), for example about 0.1 to 5 mg, for example about: 0.1, 0.25, 0.5, 0.75, 1.5, or 3.0 mg, about 0.001 mg to about 10 mg of a vasodilator (e.g., phentolamine), for example about 0.1 to 5 mg, or about 0.1 to 1 mg, for example about 0.5 mg, a pharmaceutically acceptable carrier mixture of about 1 to 50 mg, for example about 10 to 30 mg, or about 15 to 20 mg, for example about 18 mg. For making dose units, enabling agents, additives or excipients that do not interfere with the function of the active ingredients can be used.

In one aspect, a formulation herein can comprise a carrier that comprises: (i) a first crystalline cellulose at about 50% to about 89% w/w of the formulation (e.g., about: 55%-89%, 60%-89%, 65%-89%, 70%-89%, 75%-89%, 80-89%, or 85%-89%), wherein 85% of the first crystalline cellulose has a particle distribution of about 20 to about 60 microns and/or an average particle diameter of about 30 microns or less, e.g., PH-F20; (ii) a second crystalline cellulose at about 10% w/w of the formulation, wherein the second crystalline cellulose has an average particle diameter of about 150 microns or less, e.g., about 50 microns, e.g., PH-301; and (iii) tribasic calcium phosphate (TCP) at about 0.8% w/w of the formulation.

In one aspect, the formulations herein can have one or more of the following advantages: (a) comparable absorption to IM epinephrine (e.g., EpiPen®); (b) no pretreatment with phentolamine required; (c) improved solubility of epinephrine; (d) low liquid volume, e.g., 0.1 mL in each nostril; (e) tolerable to high dose epinephrine (e.g., 100 mg/animal); (f) no toxicity; (g) compatible with simple nasal delivery devices and (h) assessment can be applied to unconventional nasal models (e.g., dog).

### Method of Treating Cardiac Arrest and Hypotensive Shock

Also provided herein are methods for treating cardiac arrest in a patient undergoing cardiopulmonary resuscitation, comprising, (i) initiating cardiopulmonary resuscitation (CPR); (ii) using an automated external defibrillator (AED); and (iii) intranasally administrating an dry powder composition comprising one or more vasoactive agents (e.g., epinephrine) sufficient to increase a patient's arterial blood pressure. In another embodiment, the intranasal dry powder composition is administered if the AED fail to resume a spontaneous circulation and/or increase a patient's arterial blood pressure. In some embodiments, the administration of the intranasal dry powder composition composes a single or multiple doses.

Provided herein also are methods for treating severe hypotension in a patient undergoing hypotensive shock, comprising intranasally administrating a dry powder composition comprising one or more vasoactive agents sufficient to increase a patient's arterial blood pressure. In some embodiments, the hypotensive shock is secondary to causes including trauma, hypovolemia, severe bradycardia, or septic shock.

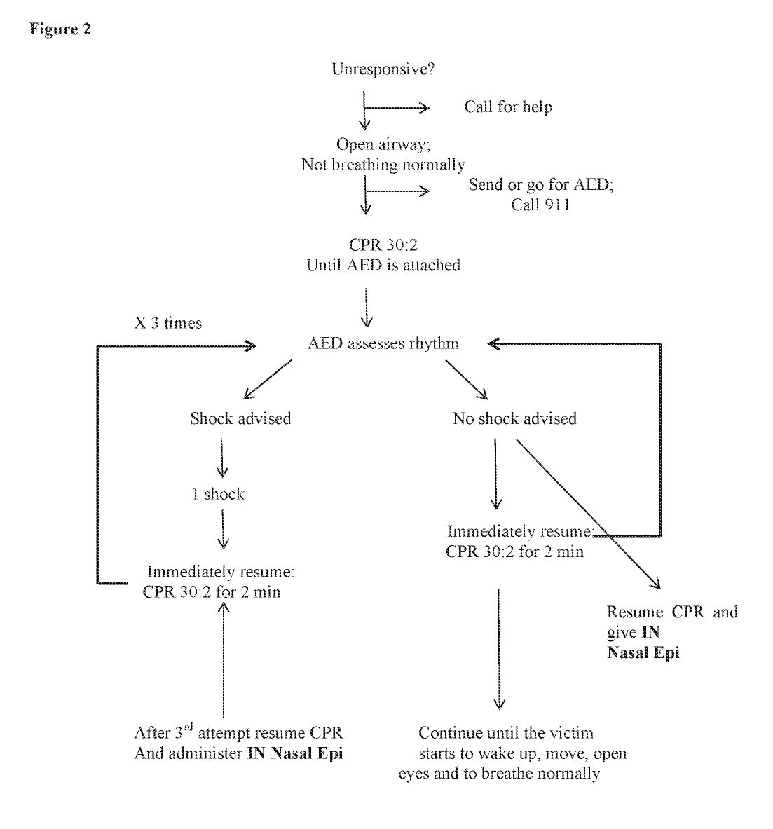

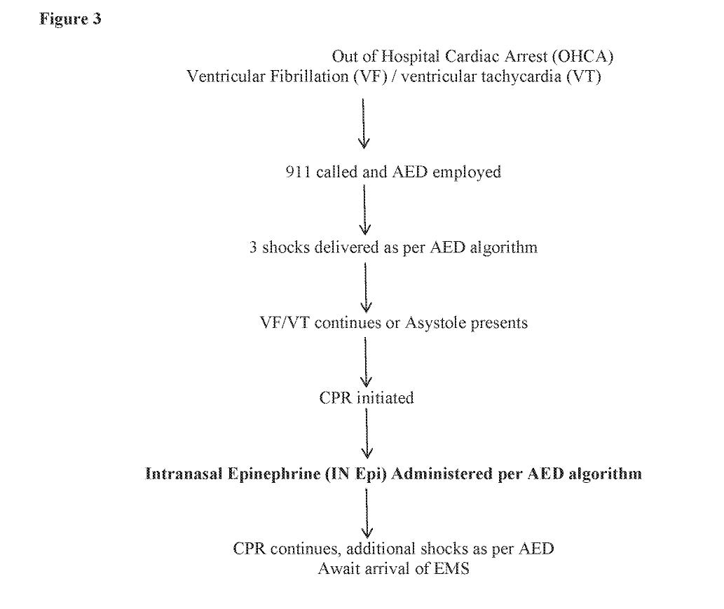

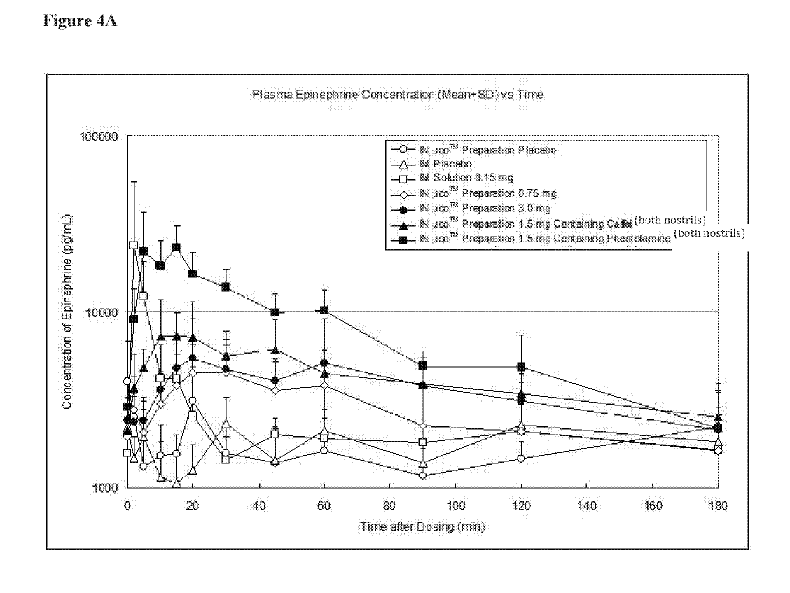

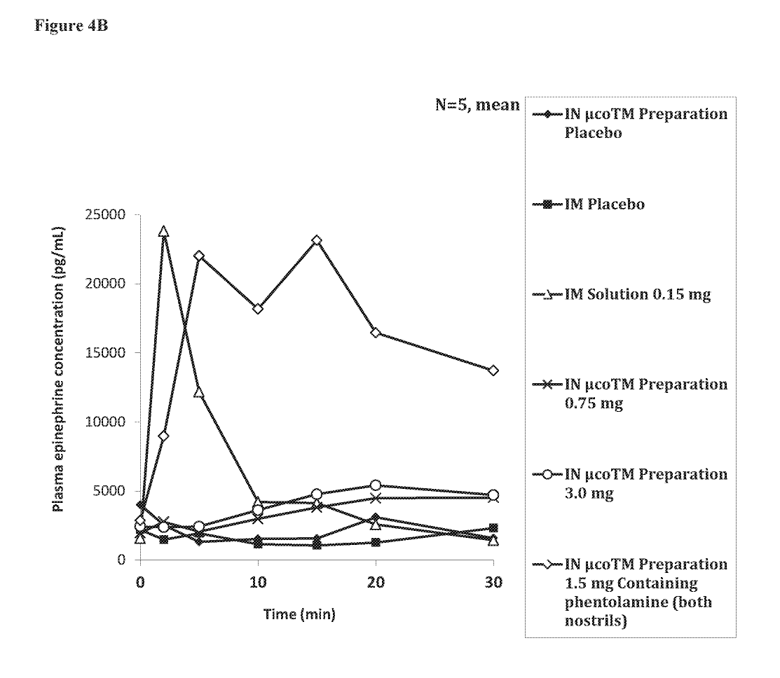

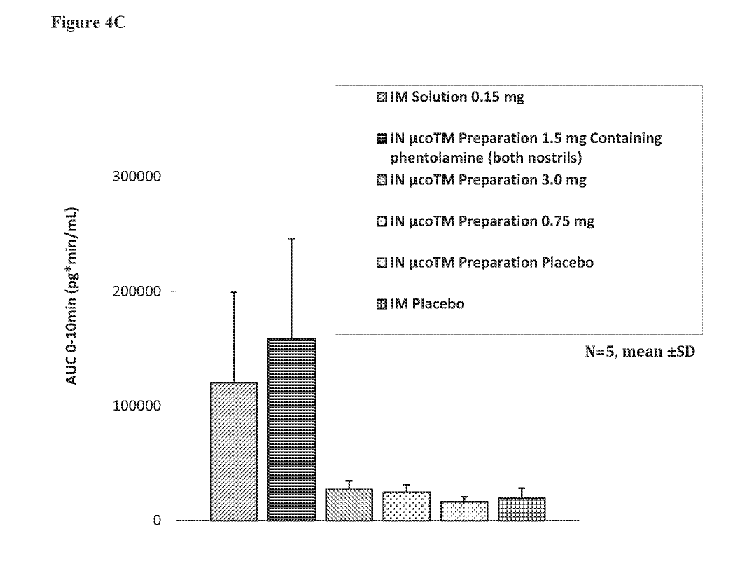

Since intranasal epinephrine is a non-technical route of administration a layperson may administer epinephrine during an emergency setting. Specifically, intranasal epinephrine may be provided in conjunction with AED units, incorporated into kits containing AEDs and epinephrine, and administration of epinephrine can be added to the algorithm for AED use. This would enable the earlier delivery of epinephrine in the critical minutes prior to arrival of professional medical personnel. In those situations where the administration of a shock by the AED fails to terminate the ventricular arrhythmia or ventricular tachycardia, nasal epinephrine can be administered to help maintain coronary perfusion pressure along with CPR until trained medical help arrives. An example of a commonly used AED algorithm modified to include nasal epinephrine is shown in FIGS. 2 and 3. Provided herein are methods of treating cardiac arrest (in or out of hospital, ER and/or in a combat setting), hypotensive shock, bradycardia, or other conditions requiring the implementation of ACLS in an individual. The methods can comprise administering an effective amount of dry powder compositions to the mucosal surfaces of the nasal cavities of an individual.

Methods of Treating Anaphylaxis and/or Anaphylactoid

Provided herein are methods of reducing an anaphylactic or anaphylactoid reaction in an individual, methods of reducing a symptom of anaphylaxis and/or anaphylactoid, methods of reducing the risk of a full-blown anaphylactic response, anaphylactoid reactions, bronchospasm, and/or cardiac arrest in an individual optionally in need thereof, and methods of reducing the incidence of the same. The methods can comprise administering an effective amount of the dry powder compositions and/or unit doses to the mucosal surfaces of the nasal cavities of an individual optionally in need thereof. The methods can be useful to treat an anaphylactic response and/or anaphylactoid reaction.

Pharmaceutical compositions, unit doses or products herein can be useful in the treatment of human conditions known to be responsive to an anti-anaphylactic and/or anti-anaphylactoid agent (e.g., epinephrine), including anaphylaxis and/or anaphylactoid reactions, and/or for rescuing a subject in bronchospasm and/or cardiac arrest. They can also provide a fast onset time and can be suitable for intranasal use.

### EXAMPLES

While some embodiments have been shown and described herein, such embodiments are provided by way of example only. Numerous variations, changes, and substitutions will now occur to those skilled in the art without departing from the invention. It should be understood that various alternatives to the embodiments of the invention described herein can be employed in practicing the invention.

While some embodiments use epinephrine as the vasoactive agent, and/or anti-anaphylactic and anti-anaphylactoid agent in the formulations and/or unit doses, some other vasoactive agent, and/or anti-anaphylactic and/or anti-anaphylactoid agent(s) can replace and/or augment epinephrine in the formulations and/or unit doses. The amount of a vasoactive agent, and/or an anti-anaphylactic and/or anti-anaphylactoid agent present in the formulations and/or unit doses can be adjusted individually as necessary to be pharmaceutically effective.

### Example 1

### Specific Aqueous Intranasal Formulation Containing Epinephrine, Entacapone and Phentolamine

To obtain the desired intranasal composition for the treatment of cardiac arrest, hypotensive shock, and/or anaphylaxis and/or anaphylactoid, a specific aqueous intranasal formulation may be formulated using nanoformulated reagents in volumes up to 250 and other vasodilators or reversible COMT inhibitors at appropriate concentrations.

An aqueous composition comprised of the following components (per 1 mL volume):

0.5-100 mg epinephrine,

1 mg polysorbate 80,

2 mg methylcellulose,

0.08 mg entacapone,

0.5 mg phentolamine hydrochloride,

sodium chloride q.s. to provide osmolality in the range of 200 to 350 mOsm/kg, and hydrochloric acid q.s. to adjust pH to 3.5-5.0.

The aqueous composition is sterile filled into a vial affixed with a nasal aqueous spray pump for delivery of 0.1 mL of atomized product per discharge at a fill volume sufficient to provide at least 2 doses.

In another embodiment of this example, an aqueous composition comprise of the following components (per 1 mL volume):

0.5 mg epinephrine maleate,

1 mg polysorbate 80,

2 mg methylcellulose,

1 mg dipivefrin hydrochloride as vasodilator,

sorbitol q.s. to provide osmolality in the range of 200 to 350 mOsm/kg, and citric acid q.s. to adjust pH to 5.0-7.0.

The aqueous composition is sterile filled into a vial affixed with a nasal spray pump for delivery of 0.1 mL of atomized product per discharge at a fill volume sufficient to provide at least 2 doses.

In certain other embodiments of this example, the epinephrine as described in the aqueous formulation above is epinephrine hydrochloride or epinephrine maleate and the amount present in 1 mL of the composition preferably is, 0.5 mg or 1.5 mg or 1.75 mg or 2.5 mg or 2.75 mg or 3.0 mg or 3.5 mg or 4.0 mg or 4.5 mg or 5.0 mg or 10.0 mg or 15.0 mg or 20.0 mg or 25.0 mg or 30.0 mg or 35.0 mg or 40.0 mg or 45.0 mg or 50.0 mg or 55.0 mg or 60.0 mg or 65.0 mg or 70.0 mg or 75.0 mg or 80.0 mg or 85.0 mg or 90.0 mg or 95.0 mg or 100 mg.

The above-described formulations may additionally contain 1 mg DDPC, 1 mg EDTA and 10 mg methyl-beta-cyclodextrin as nasal permeation enhancers.

### Example 2

### Dry Powder Formulation of Epinephrine

A 20 mg dry powder formulation is prepared from the following ingredients: 0.15 mg or 0.3 mg or 0.75 mg or 1 mg epinephrine powder (from SIGMA-ALDRICH) mixed with one or more enabling agents including 0.0001 mg or 0.1 mg entacapone (a reversible COMT inhibitor) and/or 0.0001 mg or 0.5 mg phentolamine (vasodilator) and 1.5 mg suitable carrier, for instance lactose, and suitable penetration enhancer and mucosal transit agent. The average particle diameter of each of the ingredient is within 30 p.m. The sterile formulation can then be packaged into vials or nasal spray devices for delivery into nasal mucosa of test subjects.

Testing of Dry Powder Formulation

The pharmacokinetic study of intranasal dry powder epinephrine formulations will be tested in animal models such as mongrel dogs or macaque monkeys. The animals (approximately, 5-7 in no.) will be handled in compliance with the Animal welfare Act and will be examined and evaluated at regular intervals by study personnel. Enough care will be taken to ensure no test-article related abnormalities are noticed in any experimental animal. Before the experiment, the animals will be divided into following groups
(i) control 1 (IV placebo),
(ii) control 2 (intranasal spray with carrier only)
(iii) test group 1 (IV epinephrine)
(iv) test group 2 (intranasal spray of epinephrine and suitable carrier),
(v) test group 3 (intranasal spray of epinephrine, carrier and entacapone) or
(vi) test group 4 (intranasal spray of epinephrine, carrier and phentolamine)

The drug administration protocol and induction of ventricular fibrillation will be followed. Briefly, animals in test group 1 will receive an IV injection of 1 mg and those in test group 2 will receive a 20 mg dry powder nasal spray of a composition containing, either 0.75 mg, 1.5 mg or 3.0 mg of epinephrine. Animals in test group 3 will receive 0.75 mg, 1.5 mg or 3 mg of epinephrine and 0.001 mg or 0.01 mg or 0.5 mg of entacapone while those in test group 4 will receive 0.75 mg, 1.5 mg, or 3 mg epinephrine and 0.5 mg or 0.75 mg of phentolamine. The carrier present in the composition can be lactose or any other pharmaceutically acceptable excipients or enabling agents disclosed in the present invention. Arterial blood values, mean plasma epinephrine concentration (pg/ml) and mean pressures (mm hg) will be measured before, during and after CPR. For instance, blood samples from each group will be collected before CPR, 1, 2, 3 and 4 minutes during CPR and 1-10 minutes after CPR, and the epinephrine concentrations in the blood plasma will be analyzed.

### Example 3

### Nano-Formulation of Epinephrine (Powder) for Aqueous Reconstitution Before Use

U.S. Pat. No. 7,078,057 is referenced herein in its entirety with regards to the nanoformulation method of preference. In this disclosure, fluid bed spray drying is employed to manufacture pure API into uniform and stable 60-80 nm particles. These particles can be provided for direct intranasal formulation.

In another embodiment of this example, the nanoformulated epinephrine particles are blended with larger particles of a suitable pharmaceutically acceptable carrier, for instance lactose. In one embodiment, the nanoformulated epinephrine coated lactose particles is in the size range (i.e., Dv, 50) of 10 microns to 500 microns, for example in the range of 30-100 microns, for example about 50 microns. It is desired to minimize the fraction of nanoformulated epinephrine coated lactose with size less than 10 microns, for example no more than 10% of particles with size less than 10 microns, for example no more than 1% of particles with size less than 10 microns.

In another embodiment of this example, the nanoformulated epinephrine also contains a vasodilation agent which can be incorporated into the same nanosized particles containing epinephrine, or can be produced separately as nanoparticles, then subsequently the epinephrine nanoparticles and the vasodilating agent nanoparticles can be blended as a co-mixture; both of these cases can also be envisioned to be further blended with lactose particles in order to provide a particle size suitable for nasal administration.

### Example 4

### Nano Formulation of Epinephrine (Suspension)

U.S. Pat. No. 7,078,057 is referenced herein in its entirety with regards to the nanoformulation method of preference. In this disclosure, fluid bed spray drying is employed to manufacture pure API into uniform and stable 60-80 nm particles. These particles can be further formulated as a suspension for intranasal administration at the time of use. Such a nasal reformulation device can have the aqueous and nanoformulated dry powder mix at the time of the plunger activation. The suspension milieu can contain various pharmaceutically acceptable excipients, permeation agents, mucosal transit agents, including solvents, pH agents, stabilizers, tonicifying agents, viscosity enhancing agents, dispersing agents such as surface active agents, nasal absorption enhancers, chelators, and preservatives. Alternatively, the suspension nanoformulation of epinephrine can be preservative free.

Example solvents for the suspension nanoformulation of epinephrine include water, and non-aqueous solvents such as ethanol, and low molecular weight poly (ethylene glycols). Agents for adjusting pH in suspension nanoformulation of epinephrine include buffer systems. Said buffer systems include, but are not limited to, acetate, citrate, succinate, phosphate, amino acids, and tromethamine. Pharmaceutically acceptable acid or base can be added to achieve a pH in the range of pH 3 to 9, for example in the range of pH 4 to pH 8, for example in the range of pH 7 to 8.

Example stabilizers for the suspension nanoformulation of epinephrine include sugars and other polyols, amino acids, and various antioxidant as described in detail herein.

Example tonicifying agents for the suspension nanoformulation of epinephrine includes pharmaceutically acceptable sugars and other polyols (such as, but not limited to, trehalose, mannitol, and sorbitol) and salts (such as, but not limited to, sodium chloride). The final osmolality of the formulation is adjusted with a pharmaceutically acceptable tonicifying agent, for example sodium chloride, to achieve a final in osmolality in the range of 10 to 2000 mOsm/kg, for example in the range of 50 to 1000 mOsm/kg, for example in the range of 100 to 500 mOsm/kg, for example in the range of 270 to 330 mOsm/kg (the latter representing isotonic case).

### Example 5

### Blended Aqueous Epinephrine Formulation

U.S. Pat. No. 6,702,997 teaches the production of a pulmonary liquid inhalation nebulized spray for pediatric asthmatic patients. In this example, their target concentrations for the nebulizer solution were 0.63 mg/3 mL or 1.25 mg/3 mL for pediatric dosing. In contrast, the present invention targets a highly concentrated solution of epinephrine, so that the effective dose is not 3 mL but about 100 uL. To achieve this level of solubility of the drug with the COMT inhibitor and vasodilator, mucosal transport agents, mucosal transit agents, as well as the enzymatic antioxidants (hereafter excipients), either (1) as a sterile solution bottled under nitrogen gas in opaque bottles, or (2) as a non-sterile solution with added preservatives bottled under nitrogen in opaque bottles, we have employed strategies to achieve high concentrations of the drugs in aqueous solution. These include the use of PEG, lipid and zwitterionic solubilizers, the use of alternative salts of the active drugs, as well as specific nasal mucosal tight junction permeation agents. Such agents include, but are not limited to, chelators (such as EDTA), surface-active agents (such as DDPC or Tween-20 or Tween-80), tight junction modulating peptides, and cyclodextrins.

Herein is described the process for making a single unit dose, sterile, premixed, premeasured and preservatives-free solution for the fixed dose combination (FDC) for nasal epinephrine and the enabling drugs and excipients for the treatment of cardiac arrest. In such an embodiment, the method of the present invention comprises one or more of the following steps:

(1) adding at least a therapeutically effective pediatric or, alternatively adult, amount of epinephrine and excipients in a vehicle, such as water;

(2) optionally sterilizing the solution and sealing the container.

An osmotic adjusting agent may be added to adjust the isotonicity of the solution. In one embodiment of the present invention, the solution of the present invention is isotonic. Isotonicity may be achieved by adding an osmotic adjusting agent to adjust the isotonicity of the solution from about 280 to about 320 mOsm/kg. In addition, an acid (e.g., sulfuric acid) may be added to adjust the pH of the solution to between 3.0 to about 4.0, preferably at about 3.5.

In an alternative embodiment, the nasal solution of the present invention may be prepared as follows:

(i) fitting a high density polyethylene (HDPE) or stainless steel formulation tank with a bottom drain and peristaltic recirculation system (for HDPE) or tri-blender (for stainless steel) for mixing;

(ii) filling the tank with approximately 90% of the required amount of Purified Water USP at a temperature of between 18° C. to 25° C.;

(iii) while mixing, adding sulfuric acid, Sodium Chloride USP, and at least a therapeutically effective amount of pediatric Epinephrine USP and excipients to the tank;

(iv) continue mixing until all chemical components are dissolved;

(v) adding Purified Water USP to adjust the final volume, if necessary, thus producing the epinephrine mixture.

From the formulation tank, the epinephrine mixture is pumped out through sanitary delivery lines directly into a form-fill-seal (FFS) machine. The epinephrine mixture passes through a 0.2 micron sterilizing cartridge filter, to the filling nozzles within the sterile air shower compartment, and subsequently into formed vials of glass or low density polyethylene (LDPE) that have been silkscreened to prevent light entering. The epinephrine mixture being sterile filled under nitrogen gas into the vials such that each vial contains a single unit doses per pump action of a therapeutically effective amount of epinephrine suitable for adults and children. The filled vials are then sealed. The machine may form, fill and seal the vials in a continuous operation under aseptic conditions, thus producing a sterile product. For example, cards of five filled vials are overwrapped into a protective laminated foil pouch using an auto wrapper machine. Five to twelve such pouches may then be packaged in a shelf carton, thus forming a prepackaged therapeutic system for relieving cardiac arrest, hypotensive shock and bronchospasm. An appropriate label and instructions may be added in the shelf carton.

In an alternative embodiment of the present invention, the epinephrine solution with or without the added preservatives is still filled under nitrogen gas, but not under sterile conditions.

### Example 6

### Preliminary Pharmacokinetic Study of Intranasal Epinephrine μco™ Preparations in Monkeys

### Example 1.1

### Brief Summary

Materials

Epinephrine powder (Sigma-Aldrich. Co. LLC.): median particle size=28.7 μm, D10=17.3 μm, D90=45.5 μm
Compositions of SNBL's μco™ carrier in a 20-mg formulation:
(i) the second microcrystalline cellulose (Ceolus® PH-301) is at about 10% w/w of the formulation;
(ii) tribasic calcium phosphate (TCP) is at about 0.8% w/w of the formulation; and
(iii) the first microcrystalline cellulose (Ceolus® PH-F20), at about 70% to about 89% w/w of the formulation, makes up the rest of the weight of the formulation.

| | |
|---|---|
| Test Article 1 | SNBL μco ™ Preparation Placebo |
| | (20 mg of SNBL's μco ™ carrier) |
| Test Article 2 | Intramuscular (IM) Placebo |
| | (0.3 mL of solution consisting of 1.8 mg sodium chloride, 0.5 mg sodium |
| | metabisulfite, hydrochloric acid to adjust pH within range of 2.2-5.0, and |
| | Water for Injection) |
| Test Article 3 | IM Solution 0.15 mg |
| | (0.3 mL of solution consisting of 0.15 mg epinephrine, 1.8 mg sodium |
| | chloride, 0.5 mg sodium metabisulfite, hydrochloric acid to adjust pH |
| | within range of 2.2-5.0, and Water for Injection) |
| Test Article 4 | IM Solution 0.3 mg |
| *¹ | (0.3 mL of solution consisting of 0.3 mg epinephrine, 1.8 mg sodium |
| | chloride, 0.5 mg sodium metabisulfite, hydrochloric acid to adjust pH |
| | within range of 2.2-5.0, and Water for Injection) |
| Test Article 5 | μco ™ Preparation 0.75 mg |
| | (20 mg powder preparation consisting of 0.75 mg epinephrine, 2.0 mg PH- |
| | 301, 0.16 mg TCP, and 17.09 mg PH-F20) |
| Test Article 6 | μco ™ Preparation 1.5 mg |
| *¹ | (20 mg powder preparation consisting of 1.5 mg epinephrine, 2.0 mg PH- |
| | 301, 0.16 mg TCP, and 16.34 mg PH-F20) |
| Test Article 7 | μco ™ Preparation 3.0 mg |
| | (20 mg powder preparation consisting of 3.0 mg epinephrine, 2.0 mg PH- |
| | 301, 0.16 mg TCP, and 14.84 mg PH-F20) |
| Test Article 8 | Intranasal (IN) Lactose Preparation 1.5 mg |
| *¹ | (20 mg powder preparation consisting of 1.5 mg epinephrine and 18.5 mg |
| | lactose) |
| Test Article 9 | μco ™ Preparation 1.5 mg Containing Caffeine |
| | (20 mg powder preparation consisting of 1.5 mg epinephrine, 0.5 mg |
| | caffeine, 2.0 mg PH-301, 0.16 mg TCP, and 15.84 mg PH-F20) |
| Test Article | μco ™ Preparation 1.5 mg Containing Phentolamine |
| 10 | (20 mg powder preparation consisting of 1.5 mg epinephrine, 0.5 mg |
| | phentolamine, 2.0 mg PH-301, 0.16 mg TCP, and 15.84 mg PH-F20) |

*¹ Test Article 4, 6 and 8 were formulated, but were not administered in the study and discarded.

Storage conditions: Refrigerated and protected from light in a tight container
Storage location: Test Article Refrigerator in the Test Article Depository (acceptable range: 2° C. to 8° C.)
Handling instructions: A mask, a cap, gloves, and safety glasses were worn.

Characterization and Stability

| | |
|---|---|
| Assay | Content of Epinephrine in Test Articles 3 and 4 were measured using high- |
| | performance liquid chromatography (HPLC). |
| Uniformity | Content uniformity of epinephrine in Test Articles 5 to 10 was measured using |
| | HPLC. |
| Stability | Stability testing of epinephrine in Test Articles 3, 5, 7 and 9 were conducted after |
| *² | final dosing using HPLC. |

*² Stability test for Test Article 10 was not conducted, since its characterization was conducted on the same day as its dosing.

PK Assessment

Animals Male cynomolgus monkeys, 5 animals

### Dosing

| | | Group 1 (n = 5) | | |
|---|---|---|---|---|
| | | Test Article | | Dose Level of |
| Dosing | Day | No. | Test Article | Epinephrine |
| 1 | 0 | 1 | μco ™ Preparation | 0 mg/animal |
| | | | Placebo | |
| | | | (IN, 1 nostril) | |
| 2 | 7 | 2 | IM Placebo | 0 mg/animal |
| | | | (0.5 mL saline) | |
| 3 | 14 | 3 | IM Solution 0.15 mg | 0.15 mg/animal |
| | | | (IM injection) | |
| 4 | 21 | 5 | μco ™ Preparation | 0.75 mg/animal |
| | | | 0.75 mg | |
| | | | (IN, 1 nostril) | |
| 5 | 42 | 7 | μco ™ Preparation | 3.0 mg/animal |
| | | | 3.0 mg | |
| | | | (IN, 1 nostril) | |
| 6 | 56 | 9 | μco ™ Preparation | 3.0 mg/animal |
| | | | 1.5 mg containing | |
| | | | caffeine | |
| | | | (IN, 2 nostrils) | |
| 7 | 63 | 10 | μco ™ Preparation | 3.0 mg/animal |
| | | | 1.5 mg containing | |
| | | | phentolamine | |
| | | | (IN, 2 nostrils) | |

At least 7 days were allocated between each dosing for recovery.

Sampling Blood: Pre-dose, 2, 5, 10, 15, 20, 30, 45, 60, 90, 120 and 180 minutes after dosing
(total: 12 points)

Study Results

1. Characterization and Stability of Test Articles

[Characterization Test Results]

### Test Articles 3 and 4 (IM Solution 0.15 and 0.3 mg)

| | | IM | |
|---|---|---|---|
| Test item | Specification | Solution 0.15 mg | IM Solution 0.3 mg |
| Description | Information | Colorless Solution | Colorless Solution |
| | only | | |
| Identification | 1.000 ± 0.050 | 1.003 | 1.003 |
| (RRT) | | | |
| Assay | 100.0 ± 10.0% | 101.2% | 101.5% |
| Content | RSD ≦5.0% | 0.4% | — |
| uniformity | | | |
| Judgment | | Passed | Passed |

Test Articles 5 to 7 (μco™ Preparation 0.75, 1.5 and 3.0 mg)

| | | μco ™ | μco ™ | μco ™ |
|---|---|---|---|---|
| | | Preparation | Preparation | Preparation |
| Test item | Specification | 0.75 mg | 1.5 mg | 3.0 mg |
| Description | Information | Clear | Clear | Clear |
| | only | capsules, | capsules, | capsules, |
| | | containing | containing | containing |
| | | white | white | white |
| | | powder | powder | powder |
| Identification | 1.000 ± 0.050 | 1.000 | 1.000 | 1.000 |
| (RRT) | | | | |
| Assay | 100.0 ± 10.0% | 100.0% | 99.9% | 102.5% |
| Content | RSD ≦5.0% | 1.4% | 1.3% | 1.1% |
| uniformity | | | | |
| Judgment | | Passed | Passed | Passed |

### Test Article 8 (IN Lactose Preparation 1.5 mg)

| Test item | Specification | IN Lactose Preparation 1.5 mg |
|---|---|---|
| Description | Information | Clear capsules, containing white |
| | only | powder |
| Identification | 1.000 ± 0.050 | 1.000 |
| (RRT) | | |
| Assay | 100.0 ± 10.0% | 96.1% |
| Content | RSD ≦5.0% | 1.6% |
| uniformity | | |
| Judgment | | Passed |

### Test Articles 9 and 10 (μCo™ Preparation 1.5 mg Containing Caffeine and μCo™ Preparation 1.5 mg Containing Phentolamine)

| | | | μco ™ |
|---|---|---|---|
| | | μco ™ | Preparation 1.5 mg |
| | | Preparation 1.5 mg | Containing |
| Test item | Specification | Containing Caffeine | Phentolamine |
| Description | Information | Clear capsules, | Clear capsules, |
| | only | containing white | containing white |
| | | powder | powder |
| Identification | 1.000 ± 0.050 | 1.003 | 1.001 |
| (RRT) | | | |
| Assay | 100.0 ± 10.0% | 101.2% | 100.9% |
| Content | RSD ≦5.0% | 2.0% | 1.0% |
| uniformity | | | |
| Judgment | | Passed | Passed |

[Stability Test Result]

### Test Article 3 (IM Solution 0.15 mg)

| Test item | Specification | IM Solution 0.15 mg |
|---|---|---|
| Description | Information | Colorless Solution |
| | only | |
| Identification | 1.000 ± 0.050 | 1.005 |
| (RRT) | | |
| Assay | 100.0 ± 10.0% | 99.6% |

| | |
|---|---|
| Judgment | Passed |

Test Articles 5 and 7 (μco™ Preparation 0.75 and 3.0 mg)

| | | μco ™ | μco ™ |
|---|---|---|---|
| Test item | Specification | Preparation 0.75 mg | Preparation 3.0 mg |
| Description | Information | Clear capsules, | Clear capsules, |
| | only | containing white | containing white |
| | | powder | powder |
| Identification | 1.000 ± 0.050 | 1.002 | 1.001 |
| (RRT) | | | |
| Assay | 100.0 ± 10.0% | 98.8% | 103.0% |

| | | |
|---|---|---|
| Judgment | Passed | Passed |

Test Article 9 (μco™ Preparation 1.5 mg Containing Caffeine)

| | | μco ™ Preparation |
|---|---|---|
| Test item | Specification | 1.5 mg |
| Description | Information | Containing Caffeine |
| | only | Clear capsules, |
| | | containing white |
| | | powder |
| Identification | 1.000 ± | 1.002 |
| (RRT) | 0.050 | |
| Assay | 100.0 ± | 102.8% |
| | 10.0% | |
| Judgment | | Passed |

2. Body Weight

Unit: kg

| | Test Articles | | | | | | |
|---|---|---|---|---|---|---|---|
| | | | | | | μco ™ | μco ™ |
| | | | | | | Preparation | Preparation |
| | μco ™ | | IM | μco ™ | μco ™ | 1.5 mg | 1.5 mg |
| | Preparation | IM | Solution | Preparation | Preparation | Containing | Containing |
| Animal | Placebo | Placebo | 0.15 mg | 0.75 mg | 3.0 mg | Caffeine | Phentolamine |
| No. | Day 0 | Day 7 | Day 14 | Day 21 | Day 42 | Day 56 | Day 63 |
| 1 | 4.28 | 4.31 | 4.36 | 4.46 | 4.60 | 4.66 | 4.64 |
| 2 | 4.53 | 4.56 | 4.64 | 4.67 | 4.75 | 4.63 | 4.60 |
| 3 | 4.44 | 4.58 | 4.59 | 4.53 | 4.34 | 4.30 | 4.21 |
| 4 | 4.33 | 4.35 | 4.44 | 4.48 | 4.54 | 4.53 | 4.54 |
| 5 | 4.25 | 4.36 | 4.41 | 4.47 | 4.64 | 4.69 | 4.78 |

3. Blood Sampling: All blood samples were drawn at a scheduled sampling time.

4. Clinical Signs: No test article-related abnormalities were founded in any groups.

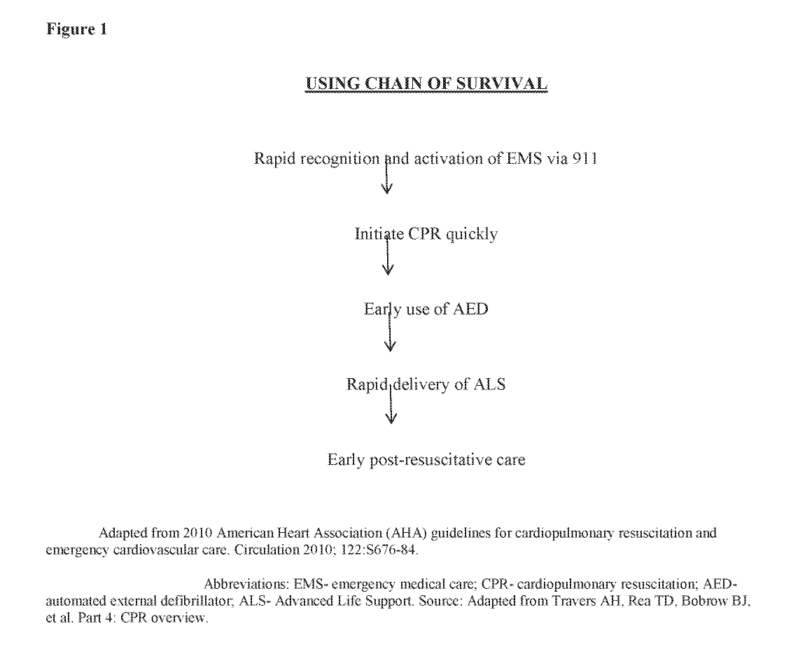

5. Pharmacokinetics: Mean plasma epinephrine concentration—time profiles and the corresponding pharmacokinetic parameters for IM Solution 0.15 mg, μco™ Preparation 0.75 mg, μco™ Preparation 3.0 mg, μco™ Preparation 1.5 mg Containing Caffeine, and μco™ Preparation 1.5 mg Containing Phentolamine, are shown in FIGS. 1A-1C and Table A, respectively.

The plasma epinephrine concentration for μco™ Preparation 1.5 mg containing phentolamine reached Cₘₐₓ of 27523 pg/mL at Tₘₐₓ of 12 minutes after dosing, while the same for IM Solution 0.15 mg reached Cₘₐₓ of 28060 pg/mL at Tₘₐₓ of 4 minutes after dosing. Mean bioavailability of epinephrine for μco™ Preparation 1.5 mg Containing Phentolamine relative to IM Solution 0.15 mg was 16.5% (calculated from AUC₍₀₋ₗₐₛₜ₎.

For the other test articles (μco™ Preparation 0.75 mg, μco™ Preparation 3.0 mg and μco™ Preparation 1.5 mg Containing Caffeine), Cₘₐₓ was lower and Tₘₐₓ was longer, compared to values of IM Solution 0.15 mg and μco™ Preparation 1.5 mg Containing Phentolamine.

TABLE A
Pharmacokinetic Parameters of Plasma Epinephrine Levels (Mean ± SD)

| | | | | | Relative | |
|---|---|---|---|---|---|---|
| | Tₘₐₓ | Cₘₐₓ | AUC₀₋ₗₐₛₜ | AUC_{0-inf} | BA₀₋ₗₐₛₜ | Relative BA_{0-inf} |
| Test articles | (min) | (pg/mL) | (pg · min/mL) | (pg · min/mL) | (%) | (%) |
| μco ™ Preparation | 40 ± 79 | 4688 ± 2504 | 298868 ± 104024 | 1542675 ± 1619207 | — | — |
| Placebo | | | | | | |
| IM Placebo | 43 ± 49 | 3658 ± 812 | 330110 ± 87439 | 1618477 ± 1470472 | — | — |
| IM Solution 0.15 mg | 4 ± 3 | 28060 ± 28061 | 458066 ± 124417 | 784325 ± 508405 | — | — |
| μco ™ Preparation 0.75 mg | 36 ± 23 | 5601 ± 2152 | 490449 ± 237626 | 833906 ± 372603 | 22.8 ± 13.8 | 26.0 ± 13.2 |
| (1 nostril) | | | | | | |
| μco ™ Preparation 3.0 mg | 27 ± 20 | 7936 ± 3604 | 652633 ± 240741 | 1152466 ± 280349 | 7.5 ± 3.6 | 9.8 ± 5.6 |
| (1 nostril) | | | | | | |
| μco ™ Preparation 1.5 mg | 17 ± 4 | 9919 ± 3744 | 763741 ± 119213 | 1359594 ± 490839 | 8.9 ± 2.9 | 11.2 ± 5.9 |
| Containing Caffeine | | | | | | |
| (total 3.0 mg, 2 nostrils) | | | | | | |
| μco ™ Preparation 1.5 mg | 12 ± 7 | 27523 ± 9902 | 1420974 ± 337369 | 1616247 ± 477769 | 16.5 ± 6.4 | 13.3 ± 7.7 |
| Containing Phentolamine | | | | | | |
| (total 3.0 mg, 2 nostrils) | | | | | | |

Each relative bioavailability (BA) values were calculated by the equation, 100 × [AUC_{(IN)} × Dose Level_{(IM)}]/[AUC_{(IM)} × Dose Level_{(IN)}].

### Example 1.2

### Detailed Methods and Results

One of the objectives for the experiments was to assess the pharmacokinetics of plasma epinephrine levels after intranasal administration with SNBL's μco™ System and compare them with intramuscular injection preparations and intranasal lactose preparation in monkeys.

1.2.1 Detailed Methods

Animal Welfare: This study was approved by the Institutional Animal Care and Use Committee (Approval No. IACUC996-148) and performed in accordance with the animal welfare bylaws of Shin Nippon Biomedical Laboratories, Ltd., Drug Safety Research Laboratories, which is fully accredited by AAALAC International.

Test and control articles: storage conditions: refrigerated, in a light-resistant and tight container; handling: a mask, a cap, gloves, and safety glasses were worn; remaining negative control article: all remaining negative control articles were discarded by the completion of the experiment.

Dosing of Test and Control Articles
Route
1st, 4th, 5th, 6th and 7th dosing: Intranasal (I.N.)
2nd and 3rd dosing: Intramuscular (I.M.)
Justification for route: In accordance with the intended clinical route
Method
I.N.: 1) Animals were placed in procedure cages, and head was held and inserted the negative pressure box contain the HEPA filter.
   2) If necessary, the nostrils were cleaned using cotton swabs before administration. One capsule containing the test article was loaded into an intranasal delivery device (Fit-lizer for monkeys, with a breath-monitoring device), and administered into the right nasal cavity by pumping the device during inhalation until the whole amount of test and control articles is completely aspirated from the capsule. The administration was confirmed by the use of the breath-monitoring device, while holding the left nostril closed. In 6th and 7th dosing, the left nostril was administered the same as the right nostril.
I.M.: Animals were placed in procedure cages. The test article was administered intramuscularly into the upper arm using a disposable needle and syringe.
Justification for method: Commonly used methods for intramuscular injection to cynomolgus monkeys. In nasal administration, using the breath-monitoring device was used in order to reduce the differences by delivery technique.
Frequency: Single dosing was performed 7 times at 1-week intervals, except between Dosing 4 and 5, which was performed 3 weeks apart, and except between Dosing 5 and 6, which was performed 2 weeks apart (2 times of negative control articles, 3 times of comparative control articles and 2 times of test articles)
Justification for frequency: To evaluate the pharmacokinetics of epinephrine after single dosing
Administration volume/amount
I.N.
1st, 4th and 5th dosing: 20 mg/animal
6th and 7th dosing: 40 mg/animal (20 mg/nostril×2)
I.M.: 0.3 mL/animal
Administration time: 09:00 to 13:00

Test System
Species: Cynomolgus monkey
   (Macaca fascicularis, purpose-bred)
Body weight (at the initiation of acclimation):
   3.5 to 5.0 kg
Age (at the initiation of acclimation):
   4 to 6 years
Origin: Cambodia
Animals for acclimation: 8 males
Animals for administration: 5 males
Justification for selection of the species: The cynomolgus monkeys, the nasal cavity of which is morphologically similar to that in humans, is commonly used as an experimental animal.

Maintenance Conditions
Room: Room No. 814
Temperature: Acceptable range: 23° C. to 29° C.
Humidity: Acceptable range: 35% to 75%
Ventilation: 15 times/hour
Illumination: 12 hours/day of artificial light (07:00 to 19:00)
Cage
Material: Stainless steel
Size: 680 mm (D)×620 mm (W)×770 mm (H)
Number of animals/cage: One
Food: Approximately 108 g (approximately 12 g×9 pieces) of solid food (HF Primate 5K91 12G 5K9J, Purina Mills, LLC) was provided to each animal daily between 14:00 and 17:00. All remaining food was removed between 08:30 and 11:00 on the following day. On the days before each dosing, any remaining food was removed at approximately 17:00, for all animals. On the each dosing day, food was provided after the final blood sampling. The analysis results for each lot of food were obtained from Purina Mills, LLC, and it was confirmed that they meet the acceptance criteria stated in the SOP.
Water: Water conforming to the water quality standards required by the Japanese waterworks law was available ad libitum from an automatic water supply system. The water is analyzed 4 times a year by Kagoshima Pharmaceutical Association. The analysis results were obtained and it was confirmed that they meet the acceptance criteria stated in the SOP.
Environmental enrichment: Enrichment toys were provided. Treats (apples or sweet potatoes) was supplied at least twice weekly (except for the days of dosing).
Cleaning: Rooms and cages (soil trays) were washed daily with water. The animals were transferred to cleaned and disinfected cages in a cleaned at least once every 4 weeks.
Airborne bacterial test: Airborne bacterial tests are conducted 4 times a year by SNBL DSR. The results were obtained, and it was confirmed that they meet the acceptance criteria stated in the SOP.

Identification of Animals and Cages
Animals: Each animal was identified by an acclimation number (ACN) marked on the chest with Animal Marker (Muromachi Kikai Co., Ltd.) or NESCO DERMARK (Alfresa Pharma Corporation) during the acclimation period. After selection of animals, each animal was identified by an animal number marked on the chest with Animal Marker (Muromachi Kikai Co., Ltd.) or NESCO DERMARK (Alfresa Pharma Corporation).
Cages: During the acclimation period, cages were identified by cage cards bearing the experiment number, ACN, sex and a bar code. After selection of animals, cages were identified by color-coded cage cards bearing the experiment number, sex, animal number and a bar code.

Acclimation

Quarantined cynomolgus monkeys (8 males) were received, and acclimated for 3 days. In order to acclimate the animals to the intranasal dosing procedure, air was administered into the right nasal cavity in the same manner as intranasal dosing once daily on Days-3, -2 and -1. Animals judged unsuitable for evaluation in the study was excluded from the study before selection of animals.

Selection of Animals

On the final day of the acclimation period, animals that showed no abnormalities during the acclimation period were selected 5 animals, and assigned from the lowest ACN. Surplus animals at selection of animals were excluded from the study on Day 1.

Maintenance Conditions
Room: Room No. 814
Temperature: Acceptable range: 23° C. to 29° C.
Humidity: Acceptable range: 35% to 75%
Ventilation: 15 times/hour
Illumination: 12 hours/day of artificial light (07:00 to 19:00)
Cage
Material: Stainless steel
Size: 680 mm (D)×620 mm (W)×770 mm (H)
Number of animals/cage: One
Food: Approximately 108 g (approximately 12 g×9 pieces) of solid food (HF Primate 5K91 12G 5K9J, Purina Mills, LLC) was provided to each animal daily between 14:00 and 17:00. All remaining food was removed between 08:30 and 11:00 on the following day. On the days before each dosing, any remaining food was removed at approximately 17:00, for all animals. On the each dosing day, food was provided after the final blood sampling. The analysis results for each lot of food were obtained from Purina Mills, LLC, and it was confirmed that they meet the acceptance criteria stated in the SOP.
Water: Water conforming to the water quality standards required by the Japanese waterworks law was available ad libitum from an automatic water supply system. The water is analyzed 4 times a year by Kagoshima Pharmaceutical Association. The analysis results were obtained and it was confirmed that they meet the acceptance criteria stated in the SOP.
Environmental enrichment: Enrichment toys were provided. Treats (apples or sweet potatoes) was supplied at least twice weekly (except for the days of dosing).
Cleaning: Rooms and cages (soil trays) were washed daily with water. The animals were transferred to cleaned and disinfected cages in a cleaned at least once every 4 weeks.
Airborne bacterial test: Airborne bacterial tests are conducted 4 times a year by SNBL DSR. The results were obtained, and it was confirmed that they meet the acceptance criteria stated in the SOP.

Identification of Animals and Cages

Each animal was identified by an acclimation number (ACN) marked on the chest with Animal Marker (Muromachi Kikai Co., Ltd.) or NESCO DERMARK (Alfresa Pharma Corporation) during the acclimation period. After selection of animals, each animal was identified by an animal number marked on the chest with Animal Marker (Muromachi Kikai Co., Ltd.) or NESCO DERMARK (Alfresa Pharma Corporation).

During the acclimation period, cages were identified by cage cards bearing the experiment number, ACN, sex and a bar code. After selection of animals, cages were identified by color-coded cage cards bearing the experiment number, sex, animal number and a bar code.

Acclimation

Quarantined cynomolgus monkeys (8 males) were received, and acclimated for 3 days. In order to acclimate the animals to the intranasal dosing procedure, air was administered into the right nasal cavity in the same manner as intranasal dosing once daily on Days-3, -2 and -1. Animals judged unsuitable for evaluation in the study was excluded from the study before selection of animals.

Selection of Animals

On the final day of the acclimation period, animals that showed no abnormalities during the acclimation period were selected 5 animals, and assigned from the lowest ACN. Surplus animals at selection of animals were excluded from the study on Day 1.

Schedule and Dosing

### First Dosing (Day 0)

| Negative Control | | Dose | Dose Amount | Number of Animals |
|---|---|---|---|---|
| Article-1 | Route | Level | (mg/animal) | (Animal Nos.) |
| IN μco ™ Preparation | I.N. | — | 20 | 5 (1 to 5) |
| Placebo | | | | |

### Second Dosing (Day 7)

| Negative Control | | Dose | Dose Volume | Number of Animals |
|---|---|---|---|---|
| Article-2 | Route | Level | (mL/animal) | (Animal Nos.) |
| IM Placebo | I.M. | — | 0.3 | 5 (1 to 5) |

### Third Dosing (Day 14)

| Comparative Control | | Dose Level | Dose Volume | Number of Animals |
|---|---|---|---|---|
| Article-1 | Route | (mg/animal) | (mL/animal) | (Animal Nos.) |
| IM Solution 0.15 mg | I.M. | 0.15 | 0.3 | 5 (1 to 5) |

### Fourth Dosing (Day 21)

| | | Dose Level | Dose Amount | Number of Animals |
|---|---|---|---|---|
| Test Article-1 | Route | (mg/animal) | (mg/animal) | (Animal Nos.) |
| IN μco ™ Preparation | I.N. | 0.75 | 20 | 5 (1 to 5) |
| 0.75 mg | | | | |

### Fifth Dosing (Day 42)

| Comparative Control | | Dose Level | Dose Volume | Number of Animals |
|---|---|---|---|---|
| Article-2 | Route | (mg/animal) | (mL/animal) | (Animal Nos.) |
| IM Solution 3.0 mg | I.N. | 3.0 | 20 | 5 (1 to 5) |

### Sixth Dosing (Day 56)

| Test | | Dose Level | Dose Amount | Number of Animals |
|---|---|---|---|---|
| Article-2 | Route | (mg/animal) | (mg/animal) | (Animal Nos.) |
| IN μco ™ Preparation | I.N. | 3.0 | 40 | 5 (1 to 5) |
| 1.5 mg containing | | (1.5 mg/ | (20 mg/nostrilx2) | |
| caffeine | | nostrilx2) | | |

### Seventh Dosing (Day 63)

| Comparative Control | | Dose Level | Dose Amount | Number of Animals |
|---|---|---|---|---|
| Article-3 | Route | (mg/animal) | (mg/animal) | (Animal Nos.) |
| IN μco ™ Preparation | I.N. | 3.0 | 40 | 5 (1 to 5) |
| 1.5 mg containing | | (1.5 mg/ | (20 mg/nostrilx2) | |
| phentolamine | | nostrilx2) | | |

Justification for Selection of the Dose Levels

For the intramuscular injection, the dose levels of epinephrine at 0.15 and 0.3 mg/animal were set according to clinical dose levels in the treatment of anaphylaxis. For intranasal formulations, the dose levels of epinephrine at 0.75, 1.5 and 3.0 mg/animal were set since low absorption is generally considered in intranasal route. These levels are not considered toxic, because they are less than 1/10,000th of the LD50 (3500 mg/kg for intramuscular administration in rats¹⁾ and 2.5 mg/kg of inhalation in rabbits²⁾). For intranasal formulations, the dose levels of caffeine at 2 mg/animal were set according to clinical dose levels in the treatment of migraine as a vasodilator, and the dose levels of phentolamine at 1 mg/animal were set according to dose levels in the treatment of cardiopulmonary arrest coadministered with epinephrine in dog (0.25 mg/kg/nostril)³⁾. These levels are not considered toxic, because they are less than 1/100th of the IVN-MUS LD50 (62 mg/kg in caffeine⁴⁾ and 75 mg/kg in phentolamine⁵⁾). Additionally, intranasal and intramuscular placebo dosings were set to evaluate changes in endogenous epinephrine by the administration and blood sampling procedures.
Material Safety Data Sheet in L-adrenaline of Nacalai Tesque, Inc.
GU X et al, Biopharm Drug Dispos, 20: 401-405, 1999
Bleske B E, et al. Am J Emerg Med. 14(2)133-138, 1996
Material Safety Data Sheet in Caffeine Citrate Injection of Luitpold Pharmaceuticals, Inc.
Material Safety Data Sheet in Phentolamine Mesylate of Tokyo Chemical Industry Co., Ltd.

Observations and Examinations

Clinical Signs
Number of animals: All
Frequency
Acclimation period: At least once daily
Dosing days: At least 4 times daily (before dosing, between immediately after dosing and 30 minutes after dosing, and approximately 1 and 4 hours after dosing)
Non-dosing days: At least once daily
Method: The animals were observed for mortality and clinical signs.

Body Weight
Number of animals: All
Frequency
Acclimation period: Once on the first and final days of acclimation
Dosing period: Before dosing on each dosing day
Method: Animals was weighed with an electronic balance (HP-40K, A&D Co., Ltd.).

Pharmacokinetics
Number of animals: All
Frequency: Each dosing day
Sampling points: Before dosing, 2, 5, 10, 15, 20, 30, 45, 60, 90, 120 and 180 minutes after dosing (total: 12 points)
Sampling volume: Approximately 1.2 mL (0.5 mL as plasma) at each sampling point
Sampling method: Blood was drawn from the femoral vein with a syringe containing heparin sodium. The blood was immediately cooled on ice and then centrifuged (4° C., 1710×g, 3000 rpm, 15 minutes). 0.5 mL of plasma and 1.0 mL of PBS was taken into a tube and mixed using a vortex mixer. The plasma was stored in a deep freezer (acceptable range: −70° C. or below).
Shipment: Frozen plasma samples were delivered to SNBL NDS (on dry ice).
Analyte: Epinephrine

1.2.2 Detailed Results

Serum samples were obtained for evaluation pharmacokinetics of Epinephrine delivered by the μco™ system compared them with intramuscular injection and intranasal lactose preparations.
1) Clinical signs (Table 3): No abnormalities were observed in any animal after each dosing.
2) Body weight (Table 4): No abnormal changes were noted in any animals on each dosing.
3) Dosing and sampling times and hemolysis (Table 5): Blood samplings were performed appropriately as scheduled. No abnormalities were observed in hemolysis of sampled plasma.
4) Pharmacokinetics (Table 6)

Study Summary

| | GROUP 1 (N = 5) | |
|---|---|---|
| | | Dose Level as |
| DOSING | Test Article | Epinephrine |
| 1 | μco ™ Preparation Placebo (IN, single nostril) | 0 mg/animal |
| 2 | IM Placebo (0.5 mL saline) | 0 mg/animal |
| 3 | IM Solution 0.15 mg (IM injection) | 0.15 mg/animal |
| 4 | μco ™ Preparation 0.75 mg (IN, single nostril) | 0.75 mg/animal |
| 5 | μco ™ Preparation 3.0 mg (IN, single nostril) | 3.0 mg/animal |
| 6 | μco ™ Preparation 1.5 mg containing caffeine | 3.0 mg/animal |
| | (IN, both nostril) | |
| 7 | μco ™ Preparation 1.5 mg containing | 3.0 mg/animal |
| | phentolamine (IN, both nostril) | |

The plasma epinephrine concentration for μco™ Preparation 1.5 mg Containing Phentolamine reached Cₘₐₓ of 27523 pg/mL at Tₘₐₓ of 12 minutes after dosing, while the same for IM Solution 0.15 mg reached Cₘₐₓ of 28060 pg/mL at Tₘₐₓ of 4 minutes after dosing. Mean bioavailability of epinephrine for μco™ Preparation 1.5 mg Containing Phentolamine relative to IM Solution 0.15 mg was 16.5% (calculated from AUC₀₋ₗₐₛₜ).

For the other test articles (μco™ Preparation 0.75 mg, μco™ Preparation 3.0 mg and μco™ Preparation 1.5 mg Containing Caffeine), compared to values of IM Solution 0.15 mg and μco™ Preparation 1.5 mg Containing Phentolamine, Cₘₐₓ was lower and Tₘₐₓ was longer.

TABLE 3
Clinical signs in male cynomolgus monkeys

| | | | Dose of | Animal | Before | Time after Dosing | | |
|---|---|---|---|---|---|---|---|---|
| Test Articles | Day | Dosing Route | Epinephrine | No. | Dosing | 30 min | 1 h | 4 h |
| μco ™ | Day 0 | Intranasal | 0 | 1 | — | — | — | — |
| Preparation | | | | 2 | — | — | — | — |
| Placebo | | | | 3 | — | — | — | — |
| | | | | 4 | — | — | — | — |
| | | | | 5 | — | — | — | — |
| IM Placebo | Day 7 | Intramuscular | 0 | 1 | — | — | — | — |
| | | | | 2 | — | — | — | — |
| | | | | 3 | — | — | — | — |
| | | | | 4 | — | — | — | — |
| | | | | 5 | — | — | — | — |
| IM Solution | Day | Intramuscular | 0.15 mg/animal | 1 | — | — | — | — |
| 0.15 mg | 14 | | | 2 | — | — | — | — |
| | | | | 3 | — | — | — | — |
| | | | | 4 | — | — | — | — |
| | | | | 5 | — | — | — | — |
| μco ™ | Day 21 | Intranasal | 0.75 mg/animal | 1 | — | — | — | — |
| Preparation | | | | 2 | — | — | — | — |
| 0.75 mg | | | | 3 | — | — | — | — |
| | | | | 4 | — | — | — | — |
| | | | | 5 | — | — | — | — |
| μco ™ | Day 42 | Intranasal | 3.0 mg/animal | 1 | — | — | — | — |
| Preparation | | | | 2 | — | — | — | — |
| 3.0 mg | | | | 3 | — | — | — | — |
| | | | | 4 | — | — | — | — |
| | | | | 5 | — | — | — | — |
| μco ™ | Day 56 | Intranasal | 3.0 | 1 | — | — | — | — |
| Preparation | | | (1.5 mg/nostril × 2 | 2 | — | — | — | — |
| 1.5 mg | | | nostrils) | 3 | — | — | — | — |
| Containing | | | | 4 | — | — | — | — |
| Caffeine | | | | 5 | — | — | — | — |
| μco ™ | Day | Intranasal | 3.0 | 1 | — | — | — | — |
| Preparation | 63 | | (1.5 mg/nostril × 2 | 2 | — | — | — | — |
| 1.5 mg | | | nostrils) | 3 | — | — | — | — |
| Containing | | | | 4 | — | — | — | — |
| Phentolamine | | | | 5 | — | — | — | — |

—: No abnormal signs

TABLE 4
Body weight (Unit: kg) in male cynomolgus monkeys

| | | Test Articles | | | | | | |
|---|---|---|---|---|---|---|---|---|
| | | | | | | | μco ™ | μco ™ |
| | | | | | | | Preparation | Preparation |
| | | μco ™ | | IM | μco ™ | μco ™ | 1.5 mg | 1.5 mg |
| | | Preparation | IM | Solution | Preparation | Preparation | Containing | Containing |
| Animal | | Placebo | Placebo | 0.15 mg | 0.75 mg | 3.0 mg | Caffeine | Phentolamine |
| No. | Day −1 | Day 0 | Day 7 | Day 14 | Day 21 | Day 42 | Day 56 | Day 63 |
| 1 | 4.34 | 4.28 | 4.31 | 4.36 | 4.46 | 4.60 | 4.66 | 4.64 |
| 2 | 4.49 | 4.53 | 4.56 | 4.64 | 4.67 | 4.75 | 4.63 | 4.60 |
| 3 | 4.47 | 4.44 | 4.58 | 4.59 | 4.53 | 4.34 | 4.30 | 4.21 |
| 4 | 4.34 | 4.33 | 4.35 | 4.44 | 4.48 | 4.54 | 4.53 | 4.54 |
| 5 | 4.34 | 4.25 | 4.36 | 4.41 | 4.47 | 4.64 | 4.69 | 4.78 |

TABLE 5-1
Results of dosing time and blood sampling time

| | | | | Blood | | | | | | |
|---|---|---|---|---|---|---|---|---|---|---|
| | | | | Sampling | | | | | | |
| | | | | Time | | Blood Sampling Time | | | | |
| | | | Animal | Before | Dosing | Time after Dosing | | | | |
| Test Articles | Day | Dosing Route | No. | Dosing | Time | 2 min | 5 min | 10 min | 15 min | 20 min |
| μco ™ | Day 0 | Intranasal | 1 | 9:10 | 9:31 | 9:33 | 9:36 | 9:41 | 9:46 | 9:51 |
| Preparation | | | 2 | 9:12 | 9:33 | 9:35 | 9:38 | 9:43 | 9:48 | 9:53 |
| Placebo | | | 3 | 9:15 | 9:36 | 9:38 | 9:41 | 9:46 | 9:51 | 9:56 |
| | | | 4 | 9:17 | 9:39 | 9:41 | 9:44 | 9:49 | 9:54 | 9:59 |
| | | | 5 | 9:18 | 9:41 | 9:43 | 9:46 | 9:51 | 9:56 | 10:01 |
| IM Placebo | Day 7 | Intramuscular | 1 | 9:19 | 9:36 | 9:38 | 9:41 | 9:46 | 9:51 | 9:56 |
| | | | 2 | 9:21 | 9:38 | 9:40 | 9:43 | 9:48 | 9:53 | 9:58 |
| | | | 3 | 9:23 | 9:40 | 9:42 | 9:45 | 9:50 | 9:55 | 10:00 |
| | | | 4 | 9:21 | 9:42 | 9:44 | 9:47 | 9:52 | 9:57 | 10:02 |
| | | | 5 | 9:19 | 9:44 | 9:46 | 9:49 | 9:54 | 9:59 | 10:04 |
| IM Solution | Day | Intramuscular | 1 | 9:21 | 9:30 | 9:32 | 9:35 | 9:40 | 9:45 | 9:50 |
| 0.15 mg | 14 | | 2 | 9:22 | 9:32 | 9:34 | 9:37 | 9:42 | 9:47 | 9:52 |
| | | | 3 | 9:24 | 9:34 | 9:36 | 9:39 | 9:44 | 9:49 | 9:54 |
| | | | 4 | 9:24 | 9:36 | 9:38 | 9:41 | 9:46 | 9:51 | 9:56 |
| | | | 5 | 9:26 | 9:38 | 9:40 | 9:43 | 9:48 | 9:53 | 9:58 |

| | | | | Blood Sampling Time | | | | | |
|---|---|---|---|---|---|---|---|---|---|
| | | | Animal | Time after Dosing | | | | | |
| Test Articles | Day | Dosing Route | No. | 30 min | 45 min | 60 min | 90 min | 120 min | 180 min |
| μco ™ Preparation | Day 0 | Intranasal | 1 | 10:01 | 10:16 | 10:31 | 11:01 | 11:31 | 12:31 |
| Placebo | | | 2 | 10:03 | 10:18 | 10:33 | 11:03 | 11:33 | 12:33 |
| | | | 3 | 10:06 | 10:21 | 10:36 | 11:06 | 11:36 | 12:36 |
| | | | 4 | 10:09 | 10:24 | 10:39 | 11:09 | 11:39 | 12:39 |
| | | | 5 | 10:11 | 10:26 | 10:41 | 11:11 | 11:41 | 12:41 |
| IM Placebo | Day 7 | Intramuscular | 1 | 10:06 | 10:21 | 10:36 | 11:06 | 11:36 | 12:36 |
| | | | 2 | 10:08 | 10:23 | 10:38 | 11:08 | 11:38 | 12:38 |
| | | | 3 | 10:10 | 10:25 | 10:40 | 11:10 | 11:40 | 12:40 |
| | | | 4 | 10:12 | 10:27 | 10:42 | 11:12 | 11:42 | 12:42 |
| | | | 5 | 10:14 | 10:29 | 10:44 | 11:14 | 11:44 | 12:44 |
| IM Solution | Day | Intramuscular | 1 | 10:00 | 10:15 | 10:30 | 11:00 | 11:30 | 12:30 |
| 0.15 mg | 14 | | 2 | 10:02 | 10:17 | 10:32 | 11:02 | 11:32 | 12:32 |
| | | | 3 | 10:04 | 10:19 | 10:34 | 11:04 | 11:34 | 12:34 |
| | | | 4 | 10:06 | 10:21 | 10:36 | 11:06 | 11:36 | 12:36 |
| | | | 5 | 10:08 | 10:23 | 10:38 | 11:08 | 11:38 | 12:38 |

TABLE 5-2
Results of dosing time and blood sampling time

| | | | | Blood | | | | | | |
|---|---|---|---|---|---|---|---|---|---|---|
| | | | | Sampling | | | | | | |
| | | | | Time | | Blood Sampling Time | | | | |
| | | Dosing | Animal | Before | Dosing | Time after Dosing | | | | |
| Test Articles | Day | Route | No. | Dosing | Time | 2 min | 5 min | 10 min | 15 min | 20 min |
| μco ™ Preparation | Day | Intranasal | 1 | 9:05 | 9:34 | 9:36 | 9:39 | 9:44 | 9:49 | 9:54 |
| 0.75 mg | 21 | | 2 | 9:07 | 9:37 | 9:39 | 9:42 | 9:47 | 9:52 | 9:57 |
| | | | 3 | 9:09 | 9:39 | 9:41 | 9:44 | 9:49 | 9:54 | 9:59 |
| | | | 4 | 9:11 | 9:42 | 9:44 | 9:47 | 9:52 | 9:57 | 10:02 |
| | | | 5 | 9:13 | 9:45 | 9:47 | 9:50 | 9:55 | 10:00 | 10:05 |
| μco ™ Preparation | Day | Intranasal | 1 | 9:09 | 9:31 | 9:33 | 9:36 | 9:41 | 9:46 | 9:51 |
| 3.0 mg | 42 | | 2 | 9:11 | 9:35 | 9:37 | 9:40 | 9:45 | 9:50 | 9:55 |
| | | | 3 | 9:13 | 9:37 | 9:39 | 9:42 | 9:47 | 9:52 | 9:57 |
| | | | 4 | 9:14 | 9:39 | 9:41 | 9:44 | 9:49 | 9:54 | 9:59 |
| | | | 5 | 9:15 | 9:41 | 9:43 | 9:46 | 9:51 | 9:56 | 10:01 |

| | | | | Blood Sampling Time | | | | | |
|---|---|---|---|---|---|---|---|---|---|
| | | Dosing | Animal | Time after Dosing | | | | | |
| Test Articles | Day | Route | No. | 30 min | 45 min | 60 min | 90 min | 120 min | 180 min |
| μco ™ Preparation | Day | Intranasal | 1 | 10:04 | 10:19 | 10:34 | 11:04 | 11:34 | 12:34 |
| 0.75 mg | 21 | | 2 | 10:07 | 10:22 | 10:37 | 11:07 | 11:37 | 12:37 |
| | | | 3 | 10:09 | 10:24 | 10:39 | 11:09 | 11:39 | 12:39 |
| | | | 4 | 10:12 | 10:27 | 10:42 | 11:12 | 11:42 | 12:42 |
| | | | 5 | 10:15 | 10:30 | 10:45 | 11:15 | 11:45 | 12:45 |
| μco ™ Preparation | Day | Intranasal | 1 | 10:01 | 10:16 | 10:31 | 11:01 | 11:31 | 12:31 |
| 3.0 mg | 42 | | 2 | 10:05 | 10:20 | 10:35 | 11:05 | 11:35 | 12:35 |
| | | | 3 | 10:07 | 10:22 | 10:37 | 11:07 | 11:37 | 12:37 |
| | | | 4 | 10:09 | 10:24 | 10:39 | 11:09 | 11:39 | 12:39 |
| | | | 5 | 10:11 | 10:26 | 10:41 | 11:11 | 11:41 | 12:41 |

TABLE 5-3
Results of dosing time and blood sampling time

| | | | | Blood | | | | | | |
|---|---|---|---|---|---|---|---|---|---|---|
| | | | | Sampling | | | | | | |
| | | | | Time | | Blood Sampling Time | | | | |
| | | Dosing | Animal | Before | Dosing | Time after Dosing | | | | |
| Test Articles | Day | Route | No. | Dosing | Time | 2 min | 5 min | 10 min | 15 min | 20 min |
| μco ™ Preparation | Day | Intranasal | 1 | 9:00 | 9:35 | 9:37 | 9:40 | 9:45 | 9:50 | 9:55 |
| 1.5 mg Containing | 56 | | 2 | 9:02 | 9:40 | 9:42 | 9:45 | 9:50 | 9:55 | 10:00 |
| Caffeine | | | 3 | 9:06 | 9:43 | 9:45 | 9:48 | 9:53 | 9:58 | 10:03 |
| (1.5 mg/nostril × 2 | | | 4 | 9:08 | 9:46 | 9:48 | 9:51 | 9:56 | 10:01 | 10:06 |
| nostrils) | | | 5 | 9:09 | 9:51 | 9:53 | 9:56 | 10:01 | 10:06 | 10:11 |
| μco ™ Preparation | Day | Intranasal | 1 | 9:22 | 9:38 | 9:40 | 9:43 | 9:48 | 9:53 | 9:58 |
| 1.5 mg Containing | 63 | | 2 | 9:23 | 9:42 | 9:44 | 9:47 | 9:52 | 9:57 | 10:02 |
| Phentolamine | | | 3 | 9:24 | 9:45 | 9:47 | 9:50 | 9:55 | 10:00 | 10:05 |
| (1.5 mg/nostril × 2 | | | 4 | 9:25 | 9:49 | 9:51 | 9:54 | 9:59 | 10:04 | 10:09 |
| nostrils) | | | 5 | 9:29 | 9:52 | 9:54 | 9:57 | 10:02 | 10:07 | 10:12 |

| | | | | Blood Sampling Time | | | | | |
|---|---|---|---|---|---|---|---|---|---|
| | | Dosing | Animal | Time after Dosing | | | | | |
| Test Articles | D ay | Route | No. | 30 min | 45 min | 60 min | 90 min | 120 min | 180 min |
| μco ™ Preparation | Day | Intranasal | 1 | 10:05 | 10:20 | 10:35 | 11:05 | 11:35 | 12:35 |
| 1.5 mg Containing | 56 | | 2 | 10:10 | 10:25 | 10:40 | 11:10 | 11:40 | 12:40 |
| Caffeine | | | 3 | 10:13 | 10:28 | 10:43 | 11:13 | 11:43 | 12:43 |
| (1.5 mg/nostril × 2 | | | 4 | 10:16 | 10:31 | 10:46 | 11:16 | 11:46 | 12:46 |
| nostrils) | | | 5 | 10:21 | 10:36 | 10:51 | 11:21 | 11:51 | 12:51 |
| μco ™ Preparation | Day | Intranasal | 1 | 10:08 | 10:23 | 10:38 | 11:08 | 11:38 | 12:38 |
| 1.5 mg Containing | 63 | | 2 | 10:12 | 10:27 | 10:42 | 11:12 | 11:42 | 12:42 |
| Phentolamine | | | 3 | 10:15 | 10:30 | 10:45 | 11:15 | 11:45 | 12:45 |
| (1.5 mg/nostril × 2 | | | 4 | 10:19 | 10:34 | 10:49 | 11:19 | 11:49 | 12:49 |
| nostrils) | | | 5 | 10:22 | 10:37 | 10:52 | 11:22 | 11:52 | 12:52 |

TABLE 5-4
Grade of hemolysis in plasma

| | | | | Hemolysis | | | | | | | | | | | |
|---|---|---|---|---|---|---|---|---|---|---|---|---|---|---|---|
| | | | | | Time after Dosing | | | | | | | | | | |
| Test | | Dosing | Animal | Before | | | | | | 30 | 45 | 60 | 90 | 120 | 180 |
| Articles | Day | Route | No. | Dosing | 2 min | 5 min | 10 min | 15 min | 20 min | min | min | min | min | min | min |
| μco ™ | Day 0 | Intranasal | 1 | N | ± | N | N | ± | + | ± | ± | + | ± | ± | N |
| Preparation | | | 2 | N | N | N | N | N | N | N | N | N | N | N | N |
| Placebo | | | 3 | N | N | N | N | N | N | N | N | N | N | N | ± |
| | | | 4 | N | N | N | N | N | N | N | N | N | N | N | N |
| | | | 5 | N | N | N | N | N | N | N | ± | N | N | N | N |
| IM | Day 7 | Intramuscular | 1 | N | N | N | N | N | N | N | N | N | N | N | N |
| Placebo | | | 2 | N | N | N | ± | ± | ± | ± | ± | ± | ± | ± | ± |
| | | | 3 | N | N | N | N | N | N | N | ± | N | ± | ± | N |
| | | | 4 | N | N | ± | N | N | ± | ± | ± | ± | ± | ± | N |
| | | | 5 | N | N | N | N | N | N | N | N | N | N | ± | N |
| IM | Day | Intramuscular | 1 | N | N | N | N | N | N | ± | N | N | N | N | ± |
| Solution | 14 | | 2 | N | N | N | ± | ± | N | ± | N | ± | ± | N | N |
| 0.15 mg | | | 3 | N | N | N | N | N | N | N | N | N | N | ± | N |
| | | | 4 | N | N | N | ± | ± | ± | + | ± | ± | N | ± | ± |
| | | | 5 | N | N | N | N | N | N | N | N | N | N | N | N |
| μco ™ | Day | Intranasal | 1 | N | N | N | N | N | N | N | N | N | N | N | N |
| Preparation | 21 | | 2 | N | N | N | N | N | N | N | N | ± | N | N | N |
| 0.75 mg | | | 3 | N | N | N | N | N | N | N | N | N | N | N | N |
| | | | 4 | N | N | N | N | N | N | N | ± | N | ± | ± | ± |
| | | | 5 | N | N | N | N | N | N | N | N | N | N | N | N |
| μco ™ | Day | Intranasal | 1 | N | N | N | N | N | N | ± | N | ± | ± | N | N |
| Preparation | 42 | | 2 | N | N | N | N | ± | ± | ± | ± | ± | ± | N | N |
| 3.0 mg | | | 3 | N | N | N | N | N | N | N | N | ± | N | N | N |
| | | | 4 | N | N | N | N | N | ± | ± | N | N | N | N | N |
| | | | 5 | N | N | N | N | N | N | N | ± | N | N | N | N |
| μco ™ | Day | Intranasal | 1 | N | N | N | N | N | N | N | N | ± | N | N | ± |
| Preparation | 56 | | 2 | N | N | N | N | N | ± | ± | N | N | ± | N | + |
| 1.5 mg | | | 3 | N | N | N | N | N | N | N | N | N | N | N | N |
| Containing | | | 4 | N | N | N | N | ± | N | ± | N | N | N | N | + |
| Caffeine | | | 5 | N | N | N | N | N | N | N | ± | N | N | N | ± |
| (1.5 mg/nostril × | | | | | | | | | | | | | | | |
| 2) | | | | | | | | | | | | | | | |
| μco ™ | Day | Intranasal | 1 | N | N | ± | N | N | ± | ± | N | ± | N | ± | N |
| Preparation | 63 | | 2 | N | N | N | N | N | N | N | N | ± | N | N | N |
| 1.5 mg | | | 3 | N | N | N | N | N | ± | N | N | N | N | N | N |
| Containing | | | 4 | N | N | N | N | N | N | N | N | N | N | N | N |
| Phentolamine | | | 5 | N | N | N | N | N | ± | N | N | N | N | N | N |
| (1.5 mg/nostril × | | | | | | | | | | | | | | | |
| 2) | | | | | | | | | | | | | | | |

Grade N: Normal ±: Slight +: Moderate

TABLE 6-1

| | | | | | Concentration of epinephrine (pg/mL) | | | | | |
|---|---|---|---|---|---|---|---|---|---|---|
| Test | | Dosing | Dose Level | Animal | Before | Time after dosing (min) | | | | |
| Article | Day | Route | (mg/animal) | No. | dosing | 2 | 5 | 10 | 15 | 20 |

Plasma epinephrine concentrations in monkeys (1st, 2nd and 3rd dosing)

| | | | | | | | | | | |
|---|---|---|---|---|---|---|---|---|---|---|
| μco ™ | Day 0 | Intranasal | — | 1 | 7911 | 5403 | 1797 | 1566 | 1641 | 3459 |
| Preparation | | | | 2 | 2727 | 2514 | 1803 | 2631 | 1587 | 1800 |
| Placebo | | | | 3 | 2253 | 2493 | 1431 | 1446 | 1794 | 3345 |
| | | | | 4 | 1299 | 996 | 603 | 579 | 834 | 1128 |
| | | | | 5 | 5808 | 1215 | 924 | 1422 | 1956 | 5781 |
| | | | | Mean | 4000 | 2524 | 1312 | 1529 | 1562 | 3103 |
| | | | | SD | 2763 | 1756 | 535 | 731 | 432 | 1800 |
| IM | Day 7 | Intramuscular | — | 1 | 3924 | 1761 | 990 | 831 | 843 | 1641 |
| Placebo | | | | 2 | 2121 | 915 | 2721 | 2349 | 2019 | 1854 |
| | | | | 3 | 1203 | 2112 | 1539 | 882 | 963 | 699 |
| | | | | 4 | 1491 | 1317 | 3495 | 771 | 705 | 774 |
| | | | | 5 | 2271 | 1257 | 909 | 909 | 768 | 1329 |
| | | | | Mean | 2202 | 1472 | 1931 | 1148 | 1060 | 1259 |
| | | | | SD | 1058 | 467 | 1135 | 673 | 545 | 513 |
| IM | Day 14 | Intramuscular | 0.15 | 1 | 1641 | 29286 | 19044 | 2070 | 1041 | 693 |
| Solution | | | | 2 | 2007 | 8583 | 6855 | 6840 | 7839 | 1392 |
| 0.15 mg | | | | 3 | 1128 | 75519 | 10626 | 2898 | 2568 | 4149 |
| | | | | 4 | 1590 | 3201 | 20157 | 2430 | 2841 | 1851 |
| | | | | 5 | 1500 | 2427 | 4140 | 6753 | 6315 | 4782 |
| | | | | Mean | 1573 | 23803 | 12164 | 4198 | 4121 | 2573 |
| | | | | SD | 315 | 30893 | 7179 | 2390 | 2836 | 1790 |

Plasma epinephrine concentrations in monkeys (4th and 5th dosing)

| | | | | | | | | | | |
|---|---|---|---|---|---|---|---|---|---|---|
| μco ™ | Day 21 | Intranasal | 0.75 | 1 | 1500 | 3639 | 3063 | 3426 | 3834 | 4692 |
| Preparation | | | | 2 | 2649 | 3177 | 1689 | 1581 | 3066 | 4170 |
| 0.75 mg | | | | 3 | 1206 | 2046 | 1191 | 1401 | 1851 | 2994 |
| | | | | 4 | 1842 | 2316 | 2502 | 3654 | 3303 | 2670 |
| | | | | 5 | 2643 | 2616 | 1854 | 4836 | 6984 | 7857 |
| | | | | Mean | 1968 | 2759 | 2060 | 2980 | 3808 | 4477 |
| | | | | SD | 659 | 647 | 731 | 1462 | 1919 | 2063 |
| μco ™ | Day 42 | Intranasal | 3.0 | 1 | 2298 | 3693 | 2790 | 3771 | 6870 | 6573 |
| Preparation | | | | 2 | 2976 | 2385 | 2148 | 3360 | 4530 | 4566 |
| 3.0 mg | | | | 3 | 1242 | 1350 | 1074 | 2154 | 2211 | 1953 |
| | | | | 4 | 3306 | 1659 | 3189 | 4044 | 2730 | 2583 |
| | | | | 5 | 2307 | 2814 | 2925 | 4728 | 7512 | 11379 |
| | | | | Mean | 2426 | 2380 | 2425 | 3611 | 4771 | 5411 |
| | | | | SD | 792 | 934 | 847 | 955 | 2382 | 3796 |

Plasma epinephrine concentrations in monkeys (6th and 7th dosing)

| μco ™ | Day | Intranasal | 3.0 | 1 | 3024 | 6966 | 5508 | 6903 | 8037 | 8823 |
|---|---|---|---|---|---|---|---|---|---|---|
| Preparation | 56 | | (1.5 mg/nostril × 2 | 2 | 3159 | 2538 | 5853 | 13737 | 5412 | 5073 |
| 1.5 mg | | | nostrils) | 3 | 1275 | 2487 | 2619 | 2910 | 4119 | 5043 |
| Containing | | | | 4 | 1713 | 1929 | 4731 | 3276 | 8331 | 3279 |
| Caffeine | | | | 5 | 1350 | 4440 | 5292 | 9189 | 10713 | 13659 |
| | | | | Mean | 2104 | 3672 | 4801 | 7203 | 7322 | 7175 |
| | | | | SD | 918 | 2072 | 1286 | 4488 | 2595 | 4150 |
| μco ™ | Day | Intranasal | 3.0 | 1 | 4551 | 12663 | 33354 | 25674 | 21531 | 18666 |
| Preparation | 63 | | (1.5 mg/nostril × 2 | 2 | 4047 | 4071 | 8001 | 9456 | 16185 | 9540 |
| 1.5 mg | | | nostrils) | 3 | 1611 | 8634 | 17718 | 18138 | 19251 | 22860 |
| Containing | | | | 4 | 1869 | 5289 | 9618 | 12993 | 23769 | 18792 |
| Phentolamine | | | | 5 | 2265 | 14184 | 41445 | 24627 | 35004 | 12444 |
| | | | | Mean | 2869 | 8968 | 22027 | 18178 | 23148 | 16460 |
| | | | | SD | 1338 | 4430 | 14787 | 7084 | 7198 | 5368 |
| | | | | | Concentration of epinephrine (pg/mL) | | | | | |
| Test | | Dosing | Dose Level | Animal | Time after dosing (min) | | | | | |
| Article | Day | Route | (mg/animal) | No. | 30 | 45 | 60 | 90 | 120 | 180 |

Plasma epinephrine concentrations in monkeys (1st, 2nd and 3rd dosing)

| | | | | | | | | | | |
|---|---|---|---|---|---|---|---|---|---|---|
| μco ™ | Day 0 | Intranasal | — | 1 | 1656 | 2142 | 2703 | 1209 | 1995 | 1236 |
| Preparation | | | | 2 | 2127 | 2289 | 1374 | 1500 | 1149 | 5076 |
| Placebo | | | | 3 | 1374 | 1026 | 966 | 837 | 1368 | 1134 |
| | | | | 4 | 510 | 660 | 702 | 357 | 1158 | 1134 |
| | | | | 5 | 2232 | 804 | 2373 | 1923 | 1644 | 2547 |
| | | | | Mean | 1580 | 1384 | 1624 | 1165 | 1463 | 2225 |
| | | | | SD | 692 | 772 | 876 | 602 | 359 | 1702 |
| IM | Day 7 | Intramuscular | — | 1 | 3213 | 2310 | 2064 | 957 | 1974 | 1170 |
| Placebo | | | | 2 | 1380 | 774 | 720 | 1686 | 2880 | 1590 |
| | | | | 3 | 3072 | 933 | 1173 | 1218 | 1185 | 1425 |
| | | | | 4 | 1245 | 828 | 1707 | 1452 | 2490 | 1737 |
| | | | | 5 | 2649 | 2277 | 4920 | 1590 | 2835 | 3150 |
| | | | | Mean | 2312 | 1424 | 2117 | 1381 | 2273 | 1814 |
| | | | | SD | 937 | 796 | 1649 | 295 | 708 | 776 |
| IM | Day 14 | Intramuscular | 0.15 | 1 | 1398 | 1956 | 1416 | 1779 | 918 | 783 |
| Solution | | | | 2 | 1233 | 1824 | 1518 | 1296 | 1476 | 1977 |
| 0.15 mg | | | | 3 | 1194 | 1872 | 1377 | 1632 | 2037 | 1713 |
| | | | | 4 | 1053 | 1488 | 1749 | 1734 | 1899 | 651 |
| | | | | 5 | 2298 | 2853 | 3465 | 2547 | 4092 | 3030 |
| | | | | Mean | 1435 | 1999 | 1905 | 1798 | 2084 | 1631 |
| | | | | SD | 498 | 510 | 884 | 460 | 1204 | 970 |

Plasma epinephrine concentrations in monkeys (4th and 5th dosing)

| | | | | | | | | | | |
|---|---|---|---|---|---|---|---|---|---|---|
| μco ™ | Day 21 | Intranasal | 0.75 | 1 | 3648 | 3447 | 2181 | 1533 | 1590 | 1311 |
| Preparation | | | | 2 | 5418 | 5880 | 7407 | 4758 | 5433 | 3201 |
| 0.75 mg | | | | 3 | 3162 | 2718 | 3879 | 1302 | 1176 | 1116 |
| | | | | 4 | 2004 | 1680 | 1827 | 1134 | 906 | 1557 |
| | | | | 5 | 8373 | 4191 | 3777 | 2451 | 1359 | 900 |
| | | | | Mean | 4521 | 3583 | 3814 | 2236 | 2093 | 1617 |
| | | | | SD | 2479 | 1584 | 2210 | 1499 | 1884 | 918 |
| μco ™ | Day 42 | Intranasal | 3.0 | 1 | 4764 | 5064 | 3741 | 2427 | 2235 | 1956 |
| Preparation | | | | 2 | 3780 | 5391 | 12060 | 5481 | 5757 | 3315 |
| 3.0 mg | | | | 3 | 5325 | 2961 | 2793 | 4428 | 1734 | 1851 |
| | | | | 4 | 2478 | 2451 | 2214 | 2760 | 1791 | 2154 |
| | | | | 5 | 7161 | 4467 | 4737 | 3825 | 3999 | 1419 |
| | | | | Mean | 4702 | 4067 | 5109 | 3784 | 3103 | 2139 |
| | | | | SD | 1749 | 1298 | 4002 | 1244 | 1746 | 710 |

Plasma epinephrine concentrations in monkeys (6th and 7th dosing)

| | | | | | | | | | | |
|---|---|---|---|---|---|---|---|---|---|---|
| μco ™ | Day | Intranasal | 3.0 | 1 | 6798 | 7032 | 3906 | 3528 | 5130 | 2808 |
| Preparation | 56 | | (1.5 mg/nostril × 2 | 2 | 4272 | 3099 | 3459 | 2973 | 3486 | 1734 |
| 1.5 mg | | | nostrils) | 3 | 4671 | 3570 | 3810 | 3285 | 3123 | 3444 |
| Containing | | | | 4 | 3510 | 6429 | 7215 | 6762 | 3072 | 3489 |
| Caffeine | | | | 5 | 8667 | 10248 | 3807 | 2793 | 2307 | 1173 |
| | | | | Mean | 5584 | 6076 | 4439 | 3868 | 3424 | 2530 |
| | | | | SD | 2112 | 2898 | 1561 | 1642 | 1046 | 1038 |
| μco ™ | Day | Intranasal | 3.0 | 1 | 15990 | 14799 | 14607 | 6354 | 8298 | 4416 |
| Preparation | 63 | | (1.5 mg/nostril × 2 | 2 | 9408 | 8526 | 11040 | 4680 | 6261 | 2292 |
| 1.5 mg | | | nostrils) | 3 | 18897 | 8826 | 10629 | 5550 | 3864 | 1689 |
| Containing | | | | 4 | 12255 | 9147 | 6759 | 3774 | 4128 | 1269 |
| Phentolamine | | | | 5 | 12012 | 8187 | 7482 | 4125 | 1713 | 1173 |
| | | | | Mean | 13712 | 9897 | 10103 | 4897 | 4853 | 2168 |
| | | | | SD | 3729 | 2763 | 3142 | 1055 | 2511 | 1332 |

TABLE 6-2

| | | | | | | | | | | | |
|---|---|---|---|---|---|---|---|---|---|---|---|
| | | | | | | | | | | Relative | Relative |
| Test | | | Dose Level | Animal | Tₘₐₓ | Cₘₐₓ | T_{1/2} | AUC₀₋ₗₐₛₜ | AUC_{0-inf} | BA₀₋ₗₐₛₜ | BA_{0-inf} |
| Article | Day | Dosing Route | (mg/animal) | No. | (min) | (pg/mL) | (min) | (min · pg/mL) | (min · pg/mL) | (%) | (%) |

PK parameters for plasma epinephrine concentrations in monkeys (1st, 2nd
and 3rd dosing)

| | | | | | | | | | | | |
|---|---|---|---|---|---|---|---|---|---|---|---|
| μco ™ | Day 0 | Intranasal | — | 1 | 0 | 7911 | 157 | 347357 | 627141 | — | — |
| Preparation | | | | 2 | 180 | 5076 | NC | 391637 | NC | — | — |
| Placebo | | | | 3 | 20 | 3345 | 219 | 230487 | 588642 | — | — |
| | | | | 4 | 0 | 1299 | NC | 150636 | NC | — | — |
| | | | | 5 | 0 | 5808 | 827 | 374222 | 3412242 | — | — |
| | | | | Mean | 40 | 4688 | 401 | 298868 | 1542675 | — | — |
| | | | | SD | 79 | 2504 | 370 | 104024 | 1619207 | — | — |
| IM | Day 7 | Intramuscular | — | 1 | 0 | 3924 | 125 | 306857 | 517289 | — | — |
| Placebo | | | | 2 | 120 | 2880 | NC | 323978 | NC | — | — |
| | | | | 3 | 30 | 3072 | 395 | 238509 | 1049775 | — | — |
| | | | | 4 | 5 | 3495 | NC | 306059 | NC | — | — |
| | | | | 5 | 60 | 4920 | 619 | 475145 | 3288368 | — | — |
| | | | | Mean | 43 | 3658 | 380 | 330110 | 1618477 | — | — |
| | | | | SD | 49 | 812 | 247 | 87439 | 1470472 | — | — |
| IM | Day | Intramuscular | 0.15 | 1 | 2 | 29286 | 104 | 368630 | 486402 | — | — |
| Solution | 14 | | | 2 | 2 | 8583 | 88 | 376257 | 626251 | — | — |
| 0.15 mg | | | | 3 | 2 | 75519 | 67 | 556880 | 723468 | — | — |
| | | | | 4 | 5 | 20157 | 58 | 362298 | 417100 | — | — |
| | | | | 5 | 10 | 6753 | 238 | 626265 | 1668405 | — | — |
| | | | | Mean | 4 | 28060 | 111 | 458066 | 784325 | — | — |
| | | | | SD | 3 | 28061 | 73 | 124417 | 508405 | — | — |

PK parameters for plasma epinephrine concentrations in monkeys (4th and 5th
dosing)

| | | | | | | | | | | | |
|---|---|---|---|---|---|---|---|---|---|---|---|
| μco ™ | Day | Intranasal | 0.75 | 1 | 20 | 4692 | 88 | 397587 | 563140 | 21.6 | 23.2 |
| Preparation | 21 | | | 2 | 60 | 7407 | 111 | 877695 | 1392183 | 46.7 | 44.5 |
| 0.75 mg | | | | 3 | 60 | 3879 | 435 | 342833 | 1043491 | 12.3 | 28.8 |
| | | | | 4 | 10 | 3654 | 117 | 285308 | 547501 | 15.7 | 26.3 |
| | | | | 5 | 30 | 8373 | 57 | 548822 | 623215 | 17.5 | 7.5 |
| | | | | Mean | 36 | 5601 | 162 | 490449 | 833906 | 22.8 | 26.0 |
| | | | | SD | 23 | 2152 | 155 | 237626 | 372603 | 13.8 | 13.2 |
| μco ™ | Day | Intranasal | 3.0 | 1 | 15 | 6870 | 292 | 576941 | 1401464 | 7.8 | 14.4 |
| Preparation | 42 | | | 2 | 60 | 12060 | 73 | 1013636 | 1361829 | 13.5 | 10.9 |
| 3.0 mg | | | | 3 | 30 | 5325 | 117 | 485606 | 797272 | 4.4 | 5.5 |
| | | | | 4 | 10 | 4044 | 283 | 419022 | 1297180 | 5.8 | 15.5 |
| | | | | 5 | 20 | 11379 | 67 | 767960 | 904585 | 6.1 | 2.7 |
| | | | | Mean | 27 | 7936 | 166 | 652633 | 1152466 | 7.5 | 9.8 |
| | | | | SD | 20 | 3604 | 112 | 240741 | 280349 | 3.6 | 5.6 |

PK parameters for plasma epinephrine concentrations in monkeys (6th and 7th
dose)

| | | | | | | | | | | | |
|---|---|---|---|---|---|---|---|---|---|---|---|
| μco ™ | Day | Intranasal | 3.0 | 1 | 20 | 8823 | 115 | 882614 | 1350276 | 12.0 | 13.9 |
| Preparation | 56 | | (1.5 mg/nostril × 2 | 2 | 10 | 13737 | 124 | 642501 | 951892 | 8.5 | 7.6 |
| 1.5 mg | | | nostrils) | 3 | 20 | 5043 | 303 | 631004 | 2137632 | 5.7 | 14.8 |
| Containing | | | | 4 | 15 | 8331 | 114 | 856505 | 1430951 | 11.8 | 17.2 |
| Caffeine | | | | 5 | 20 | 13659 | 72 | 806081 | 927220 | 6.4 | 2.8 |
| | | | | Mean | 17 | 9919 | 146 | 763741 | 1359594 | 8.9 | 11.2 |
| | | | | SD | 4 | 3744 | 90 | 119213 | 490839 | 2.9 | 5.9 |
| μco ™ | Day | Intranasal | 3.0 | 1 | 5 | 33354 | 70 | 1992672 | 2441126 | 27.0 | 25.1 |
| Preparation | 63 | | (1.5 mg/nostril × 2 | 2 | 15 | 16185 | 72 | 1230779 | 1469555 | 16.4 | 11.7 |
| 1.5 mg | | | nostrils) | 3 | 20 | 22860 | 52 | 1451268 | 1578256 | 13.0 | 10.9 |
| Containing | | | | 4 | 15 | 23769 | 49 | 1157834 | 1247463 | 16.0 | 15.0 |
| Phentolamine | | | | 5 | 5 | 41445 | 43 | 1272315 | 1344836 | 10.2 | 4.0 |
| | | | | Mean | 12 | 27523 | 57 | 1420974 | 1616247 | 16.5 | 13.3 |
| | | | | SD | 7 | 9902 | 13 | 337369 | 477769 | 6.4 | 7.7 |

HD 0-last: area under the curve from 0 to 180 minutes
AUC_{0-inf}: area under the curve from 0 to infinity
Relative BA₀₋ₗₐₛₜ (%): ((AUC_{[I.N.]0-last} × Dose Level_{[I.M.]})/(AUC_{[I.M.]0-last} × Dose Level_{[I.N.]})) × 100
Relative BA_{0-inf} (%): ((AUC_{[I.N.]0-inf} × Dose Level_{[I.M.]})/(AUC_{[I.M.]0-inf} × Dose Level_{[I.N.]})) × 100

### Example 7

### Preparation, Characterization and Stability of Epinephrine Preparations

Test articles of Epinephrine were prepared, had the characteristics determined, and were evaluated for their stability after use in a preliminary pharmacokinetic study

Preparations

Test Article-1
Name: IM Solution 0.15 mg
Nominal composition: 0.3 mL of solution contains 0.15 mg epinephrine
Assay: 101.2%

Test Article-2
Name: IM Solution 0.3 mg
Nominal composition: 0.3 mL of solution contains 0.3 mg epinephrine
Assay: 101.5%

Test Article-3
Name: μco™ Preparation 0.75 mg
Nominal composition: 20 mg powder preparation consisting of 0.75 mg epinephrine and SNBL's μco™ carrier
Assay: 100.0%

Test Article-4
Name: μco™ Preparation 1.5 mg
Nominal composition: 20 mg powder preparation consisting of 1.5 mg epinephrine and SNBL's μco™ carrier
Assay: 99.9%

Test Article-5
Name: IN Lactose Preparation 1.5 mg
Nominal composition: 20 mg powder preparation consisting of 1.5 mg epinephrine and lactose
Assay: 96.1%

Test Article-6
Name: μco™ Preparation 3.0 mg
Nominal composition: 20 mg powder preparation consisting of 3.0 mg epinephrine and SNBL's μco™ carrier
Assay: 102.5%

Test Article-7
Name: μco™ Preparation 1.5 mg Containing Caffeine
Nominal composition: 20 mg powder preparation consisting of 1.5 mg epinephrine, 1.0 mg caffeine and SNBL's μco™ carrier
Assay: 101.2%

Test Article-8
Name: μco™ Preparation 1.5 mg Containing Phentolamine
Nominal composition: 20 mg powder preparation consisting of 1.5 mg epinephrine, 0.5 mg phentolamine and SNBL's μco™ carrier
Assay: 100.9%

Characterization

| | | IM solution | IM solution | μco ™ Preparation |
|---|---|---|---|---|
| | | 0.15 mg | 0.3 mg | 0.75 mg |
| Test Item | Specification | Results | Results | Results |
| Description | For information | Colorless | Colorless | Clear capsules, containing |
| | only | solution | solution | white powder |
| | | Judgment: NA | Judgment: NA | Judgment: NA |
| Identification | Between 0.950 and | 1.003 | 1.003 | 1.000 |
| | 1.050 | Judgment: | Judgment: | Judgment: Passed |
| | | Passed | Passed | |
| Assay | 100% ± 10% | 101.2% | 101.5% | 100.0% |
| | | Judgment: | Judgment: | Judgment: Passed |
| | | Passed | Passed | |
| Uniformity | Not Exceeding | — | — | 1.4% |
| | 5.0% | | | Judgment: Passed |

| | | | |
|---|---|---|---|
| Overall Judgment | Passed | Passed | Passed |

| | | μco ™ | IN Lactose | μco ™ |
|---|---|---|---|---|
| | | Preparation | Preparation | Preparation |
| | | 1.5 mg | 1.5 mg | 3.0 mg |
| Test Item | Specification | Results | Results | Results |
| Description | For information | Clear capsules, | Clear capsules, | Clear capsules, |
| | only | containing white | containing white | containing white |
| | | powder | powder | powder |
| | | Judgment: NA | Judgment: NA | Judgment: NA |
| Identification | Between 0.950 | 1.000 | 1.000 | 1.000 |
| | and 1.050 | Judgment: Passed | Judgment: Passed | Judgment: Passed |
| Assay | 100% ± 10% | 99.9% | 96.1% | 102.5% |
| | | Judgment: Passed | Judgment: Passed | Judgment: Passed |
| Uniformity | Not Exceeding | 1.3% | 1.6% | 1.1% |
| | 5.0% | Judgment: Passed | Judgment: Passed | Judgment: Passed |

| | | | |
|---|---|---|---|
| Overall Judgment | Passed | Passed | Passed |

| | | μco ™ Preparation 1.5 mg | μco ™ Preparation 1.5 mg |
|---|---|---|---|
| | | Containing Caffeine | Containing Phentolamine |
| Test Item | Specification | Results | Results |
| Description | For information | Clear capsules, containing | Clear capsules, containing |
| | only | white powder | white powder |
| | | Judgment: NA | Judgment: NA |
| Identification | Between 0.950 and | 1.003 | 1.001 |
| | 1.050 | Judgment: Passed | Judgment: Passed |
| Assay | 100% ± 10% | 101.2% | 100.9% |
| | | Judgment: Passed | Judgment: Passed |
| Uniformity | Not Exceeding | 2.0% | 1.0% |
| | 5.0% | Judgment: Passed | Judgment: Passed |

| | | |
|---|---|---|
| Overall Judgment | Passed | Passed |

Stability

| | | IM solution | μco ™ Preparation | μco ™ Preparation |
|---|---|---|---|---|
| | | 0.15 mg | 0.75 mg | 3.0 mg |
| Test Item | Specification | Results | Results | Results |
| Description | For information | Colorless | Clear capsules, | Clear capsules, |
| | only | solution | containing white | containing white |
| | | Judgment: NA | powder | powder |
| | | | Judgment: NA | Judgment: NA |
| Identification | Between 0.950 | 1.005 | 1.002 | 1.001 |
| | and 1.050 | Judgment: | Judgment: Passed | Judgment: Passed |
| | | Passed | | |
| Assay | 100% ± 10% | 99.6% | 98.8% | 103.0% |
| | | Judgment: | Judgment: Passed | Judgment: Passed |
| | | Passed | | |

| | | | |
|---|---|---|---|
| Overall Judgment | Passed | Passed | Passed |

| | | μco ™ Preparation 1.5 mg |
|---|---|---|
| | | Containing Caffeine |
| Test Item | Specification | Results |
| Description | For information | Clear capsules, containing |
| | only | white powder |
| | | Judgment: NA |
| Identification | Between 0.950 | 1.002 |
| | and 1.050 | Judgment: Passed |
| Assay | 100% ± 10% | 102.8% |
| | | Judgment: Passed |

| | |
|---|---|
| Overall Judgment | Passed |

Stability testing was conducted for formulations used in the pharmacokinetic study (SBL996-148). Stability testing of μco™ Preparation 1.5 mg Containing Phentolamine was not conducted, because its characterization testing was conducted on the date of its administration.

Matrix Components

| Name | Grade |
|---|---|
| Excipient 1 | NF |
| Excipient 2 | NF |
| Excipient 3 | NF |
| Lactose(Raspitose SV003) | NF |
| (DMV INTERNATIONAL) | |
| Caffeine | Special grade |
| (Wako Pure Chemical | |
| Industries, Ltd.) | |
| Phentolamine mesylate | — |
| (TOKYO CHEMICAL | |
| INDUSTRY CO., LTD.) | |

Reagents

| Name | Grade | Manufacturer |
|---|---|---|
| Monobasic sodium | Special grade | Wako Pure Chemical Industries, Ltd. |
| phosphate | | |
| Sodium | Ion-pair | Wako Pure Chemical Industries, Ltd. |
| 1-octanesulfonate | chromatography | |
| | grade | |
| Disodium edetate | JP | Wako Pure Chemical Industries, Ltd. |
| hydrate | | |
| Phosphoric acid | Special grade | Wako Pure Chemical Industries, Ltd |
| Methanol | HPLC grade | Wako Pure Chemical Industries, Ltd |
| Sodium chloride | Special grade | Wako Pure Chemical Industries, Ltd |
| Sodium | Special grade | Wako Pure Chemical Industries, Ltd |
| metabisulfite | | |
| 1 mol/l | Special grade | Wako Pure Chemical Industries, Ltd |
| hydrochloric acid | | |
| Water for injection | JP | Otsuka Pharmaceutical Co. Ltd |

| | |
|---|---|
| Ultrapure water | Water purified using the Milli-Q system |

Other Materials

| Name | Model | Manufacturer |
|---|---|---|
| Syringe filter | DISMIC-3JP, DISMIC-13JP, | Toyo Roshi Kaisha, Ltd. |
| | DISMIC-25JP (PTFE, 0.50 μm) | |

Measurement Equipment

| Name | Model | Manufacturer |
|---|---|---|
| HPLC system | Shimadzu 20A | Shimadzu Corp. |
| UV detector | Shimadzu SPD-20A | Shimadzu Corp. |
| Data management system | LCsolution | Shimadzu Corp. |

Preparation of Test Articles

Powder Preparation

Epinephrine and three types of excipients were mixed to prepare μco™ preparation. Epinephrine, three types of excipients, and caffeine were mixed to prepare μco™ preparation containing caffeine. Epinephrine, three types of excipients, and phentolamine were mixed to prepare μco™ preparation containing phentolamine. Epinephrine and lactose were mixed to prepare the IN Lactose Preparation. The powder preparations were weighed and encapsulated in HPMC capsules (20±1 mg per capsule) to prepare the test articles. The length of each capsule was adjusted to the required size (17.8±0.4 mm) using the capsule sizer provided.

| | Nominal Content | | |
|---|---|---|---|
| | of Epinephrine/ | Fill Weight | No. of |
| Name | Capsule (mg) | per Capsule | Capsules |
| μco ™ | 0.75 mg/20 mg | 20 ± 1 mg | Approx. 20 |
| Preparation 0.75 | | | |
| mg | | | |
| μco ™ | 1.5 mg/20 mg | 20 ± 1 mg | Approx. 20 |
| Preparation 1.5 | | | |
| mg | | | |
| IN lactose | 1.5 mg/20 mg | 20 ± 1 mg | Approx. 20 |
| Preparations 1.5 | | | |
| mg | | | |
| μco ™ | 3.0 mg/20 mg | 20 ± 1 mg | Approx. 20 |
| Preparation 3.0 | | | |
| mg | | | |
| μco ™ | 1.5 mg/20 mg | 20 ± 1 mg | Approx. 25 |
| Preparation 1.5 | | | |
| mg Containing | | | |
| Caffeine | | | |
| μco ™ | 1.5 mg/20 mg | 20 ± 1 mg | Approx. 25 |
| Preparation 1.5 | | | |
| mg Containing | | | |
| Phentolamine | | | |

IM Preparation

Based on the table below, 0.3 mL of solution consisted of epinephrine, 1.8 mg sodium chloride, 0.5 mg sodium metabisulfite, hydrochloric acid to adjust pH within range of 2.2-5.0, and water for injection.

| | Final Volume | Final Concentration |
|---|---|---|
| Name | (mL) | (mg/mL) |
| IM Solution | 15 | 0.5 mg/mL |
| 0.15 mg | | |
| IM Solution | 15 | 1.0 mg/mL |
| 0.3 mg | | |

Analysis Items and Number of Replicates

Powder Preparation

| | Number of Replicate Analyses | |
|---|---|---|
| Analysis Item | Characterization | Stability |
| Description | 1 | 1 |
| Identification⁽¹⁾ | 6 | 3 |
| Assay⁽¹⁾ | 6 | 3 |
| Uniformity⁽¹⁾ | 6 | 0 |

⁽¹⁾Identification, assay and uniformity were conducted using the same solutions in the powder preparation.

IM Preparation

| | Number of Replicate Analyses | |
|---|---|---|
| Analysis Item | Characterization | Stability |
| Description | 1 | 1 |
| Identification⁽²⁾ | 3 | 3 |
| Assay⁽²⁾ | 3 | 3 |

⁽²⁾Identification and assay were conducted using the same solutions in the IM preparation.

Analysis Items

Powder Preparation

Each capsule of test article was examined and their appearance was recorded. The contents of the capsule were emptied onto a white background and record the color and form. After recording the color and form, the sample was used for assay and identification determination.

IM Preparation

The appearance of the solution was observed in front of a white background and records the color. After recording the color, the sample was used for assay and identification determination. Stability: No significant changes from the characterization were noted

Identification, Assay, Uniformity

Preparation of Mobile Phase and Reagents

Preparation of Mobile Phase A

To 1 L of 0.05 M monobasic sodium phosphate was added about 519 mg of sodium 1-octanesulfonate and about 50 mg of disodium edetate hydrate, and the solution was mixed. The pH was adjusted by the dropwise addition of phosphoric acid to a pH of 3.8. 85 volumes of this solution were mixed with 15 volumes of methanol. The solution was degassed for 10 minutes under reduced pressure and then sonicated for 5 minutes.

Preparation of Mobile Phase B

### Methanol

Preparation of Diluent

### Mobile Phase A

Preparation of Blank Sample Solution

Intranasal Powder Formulation
1. The component of BA was accurately weighed, and transferred to a volumetric flask.
2. The diluent was adjusted to about 80% of volumetric flask and sonicated for 10 minutes before diluting with the remaining diluent to target volume.
3. An aliquot was filtered through a syringe filter for analysis. Specificity solutions were prepared (n=1).

| | | Target | | Volumetric |
|---|---|---|---|---|
| Solution | Matrix Component | Weight | | flask |
| No. | [acceptable range] | (mg) | Diluent | (mL) |
| BA1 | Excipient 1 [±1%] | 136.0 | mobile phase | 100 |
| | Excipient 2 [±1%] | 16.0 | | |
| | Excipient 3 [±10%] | 2.0 | | |
| BA2 | Lactose[±1%] | 148.0 | | 200 |
| BA3 | Excipient 1 [±1%] | 122.7 | | 200 |
| | Excipient 2 [±1%] | 16.0 | | |
| | Excipient 3 [±10%] | 2.0 | | |
| | Caffeine[±10%] | 8.0 | | |
| BA4 | Excipient 1 [±1%] | 126.7 | | 200 |
| | Excipient 2 [±1%] | 16.0 | | |
| | Excipient 3 [±10%] | 2.0 | | |
| | Phentolamine | | | |
| | mesylate [±10%] | 4.0 | | |

BA1 = Equivalent to the total amount of carrier contained in 8 capsules of 0.75 mg epinephrine/20 mg intranasal powder formulation.
BA2 = Equivalent to the total amount of carrier contained in 8 capsules of 1.5 mg epinephrine/20 mg intranasal powder formulation (Lactose carrier).
BA3 = Equivalent to the total amount of carrier contained in 8 capsules of 1.5 mg epinephrine and 1.0 mg caffeine/20 mg intranasal powder formulation.
BA4 = Equivalent to the total amount of carrier contained in 8 capsules of 1.5 mg epinephrine and 0.5 mg phentolamine mesylate/20 mg intranasal powder formulation.

IM solution
1. The component of BA was accurately weighed, and transferred to a volumetric flask.
2. The diluent was adjusted to about 50% of volumetric flask and 1 mol/L hydrochloric acid to adjust pH 2.2-5.0.
3. Sonicated for 10 minutes before diluting with the remaining diluent to target volume.

| Interim | | Target | | Volumetric |
|---|---|---|---|---|
| Solution | Matrix Component | Weight | | flask |
| No. | [acceptable range] | (mg) | Diluent | (mL) |
| ba3 | Sodium chloride [±1%] | 90.0 | Water for injection | 15 |

4. In accordance with the following table, interim sample solution was diluted with the diluent (n=1).

| Sample | Sampled | Amount | | Final |
|---|---|---|---|---|
| Solution No. | from | (mL) | Diluent | volume (mL) |
| BA3 | ba3 | 3.0 | Water for injection | 25 |

5. An aliquot was filtered through a syringe filter for analysis.

Preparation of Standard Solutions

Standard solutions were prepared just before analysis in volumetric flasks.
1. In order to first prepare an original standard solution (SSA), 15 mg of epinephrine was accurately weighed, and transferred to a 50 mL volumetric flask.
2. The diluent was added and sonicated for 10 minutes before diluting with the diluent to volume.
3. SSA was prepared standard solutions at 150%, 125%, 100%, 75% and 50% of the target concentration (60 μg/mL).
4. In accordance with the following table, predetermined volume of SSA was mixed with the diluent to about 80% of the capacity of the flask.
5. Diluted with the diluent to the final volume. Mixed well.
6. An aliquot were filtered through a syringe filter for analysis.

| Standard | | | | Final | |
|---|---|---|---|---|---|
| Solution | Sampled | Amount | | volume | Concentration |
| No. | from | (mL) | Diluent | (mL) | (μg/mL) |
| SSA | — | About 15 mg | mobile phase | 50 | ca. 300 |
| S-5 | SSA | 3.0 | mobile phase | 10 | ca. 90 |
| S-4 | SSA | 2.5 | mobile phase | 10 | ca. 75 |
| S-3 | SSA | 2.0 | mobile phase | 10 | ca. 60 |
| S-2 | SSA | 3.0 | mobile phase | 20 | ca. 45 |
| S-1 | SSA | 1.0 | mobile phase | 10 | ca. 30 |

Preparation of Sample Solutions

### Sample Solutions of Intranasal Powder Formulation for Identification, Assay, Uniformity

1. Six capsules were sampled at random and the content of each capsule were emptied and transferred to an amber volumetric flask.
2. The Diluent were adjusted to about 80% of volumetric flask and sonicated for 10 minutes before diluting with the remaining diluent to target volume.
3. An aliquot were filtered through a syringe filter for analysis. Sample solutions were prepared (n=6) just before analysis (SA1-1 to -6, SA2-1 to -6, SA3-1 to -6, SA4-1 to -6, SA5-1 to -6, and SA6-1 to -6).

| Sample | | | Nominal Content | | Final | Concentration |
|---|---|---|---|---|---|---|
| Solution | | No of | of epinephrine/ | | Volume | of epinephrine |
| No. | Component | Capsules | Capsule (mg) | Diluent | (mL) | (μg/mL) |
| SA1 | Test Article 3 | 1 | 0.75 mg/20 mg | Mobile | 15 | ca.50 |
| SA2 | Test Article 4 | 1 | 1.5 mg/20 mg | phase | 25 | ca.60 |
| SA3 | Test Article 5 | 1 | 1.5 mg/20 mg | | 25 | ca.60 |
| SA4 | Test Article 6 | 1 | 3.0 mg/20 mg | | 50 | ca.60 |
| SA5 | Test Article 7 | 1 | 1.5 mg/20 mg | | 25 | ca.60 |
| SA6 | Test Article 8 | 1 | 1.5 mg/20 mg | | 25 | ca.60 |

IM Solutions for Identification, Assay
1. Sample solution was diluted with the diluent. (SA4-1 to -3 and SA5-1 to -3)
2. The diluent were adjusted to about 50% of volumetric flask and hydrochloric acid to adjust pH 2.2-5.0.
3. Sonicated for 10 minutes before diluting with the remaining diluent to target volume.

| Sample | | | | Final | |
|---|---|---|---|---|---|
| Solution | | Amount | | volume | Concentration* |
| No. | Component | (mL) | Diluent | (mL) | (μg/mL) |
| SA4 | Test Article 1 | 1.0 | Water for | 10 | ca. 50 |
| SA5 | Test Article 2 | 1.0 | injection | 20 | ca. 50 |

*The concentrations of epinephrine

4. An aliquot were filtered through a syringe filter for analysis.

HPLC Analysis Conditions
HPLC system: Shimadzu 20A
Detector: Ultraviolet absorption photometer (Shimadzu SPD-20A) Wavelength: 280 nm
Data management system: LCsolution
Column: Zorbax SB-C8 (4.6 mm i.d.×150 mm, 5 μm) Agilent Technologies
Column temperature: Set at 25° C.
Sample temperature: Set at 5° C.
Flow rate: 2.0 mL/min
Injection volume: 20 μL
HPLC Analysis Conditions of BA 4 and Test Article 8
HPLC system: Shimadzu 20A
Detector: Ultraviolet absorption photometer (Shimadzu SPD-20A) Wavelength: 280 nm
Data management system: LCsolution
Column: Zorbax SB-C8 (4.6 mm i.d.×150 mm, 5 μm) Agilent Technologies
Column temperature: Set at 25° C.
Sample temperature: Set at 5° C.
Gradient conditions: Mobile phases A and B were mixed in accordance with the following table (all changes were linear):

| Time | Mobile phase A | Mobile phase B |
|---|---|---|
| (min) | (%) | (%) |
| 0.0 | 100 | 0 |
| 4.0 | 100 | 0 |
| 4.1 | 50 | 50 |
| 8.0 | 50 | 50 |
| 8.1 | 100 | 0 |
| 15.0 | 100 | 0 |

Flow rate: 2.0 mL/min
Injection volume: 20 μL

System Suitability

System Repeatability

Five replicate injections of standard solution S-3 were performed at the beginning of analysis, one injection every 10 to 15 injections and one injection at the end of analysis. LCsolution was used to calculate the epinephrine peak areas.

The relative standard deviation (RSD) was calculated as follows:

RSD (%)=Standard deviation/mean assay×100

Acceptance criteria:
An RSD for the first five injections not exceeding 4.0%.
An RSD for all injections not exceeding 5.0%.

Determination Method

The standard solution was injected (n=1) followed by single injections of sample solutions BA1 to 4 and SA1 to 6 onto the HPLC.

Identification Evaluation

For sample solutions SA 1 to 6, the ratio of retention time was calculated as follows:

Ratio of Retention Time=Tt/Ts
Ts: Retention time of epinephrine in standard solution (S-3)
Tt: Mean retention time of principle peak in sample solution
Specification: A ratio of retention time between 0.950 and 1.050.

Assay Evaluation

Using LCsolution, a single level calibration curve was constructed from the standard solution concentration (μg/mL) and peak areas, from which the epinephrine concentration in each sample solution was obtained.

For sample solutions SA1, the epinephrine assay (%) and mean for each test article was calculated as follows:

$$Assay ( \% ) = \frac{{C}_{smp}}{1000} \times \frac{{F}_{vol}}{NC} \times 100$$

Cₛₘₚ=Concentration of epinephrine in sample solution (μg/mL)
Fᵥₒₗ=Final volume of sample solution (mL)
NC=Nominal content of epinephrine (mg)
Specification: Mean assay values within 100.0%±10.0%

Uniformity Evaluation

For sample solutions SA1, the epinephrine content per capsule was calculated as follows:

$$epinephrine content per capsule ( mg ) = \frac{{C}_{smp}}{1000} \times {F}_{vol}$$

Cₛₘₚ=Concentration of epinephrine in sample solution (μg/mL)
Fᵥₒₗ=Final volume of sample solution (mL)
Specification: An RSD not exceeding 5.0%

Evaluation

Evaluation of Characteristics

The test articles were judged to be acceptable, when the results of the assay and uniformity, and identification test items meet the specifications at the 1st analysis.

Evaluation of Stability

Stability was confirmed after storage, when the results of each test items meet the specifications. Stability study was not conducted on Test Article-2, -4, -5.

Data Handling

The slope, correlation coefficient, sample solution concentrations and system reproducibility were obtained using the data management system. Subsequent calculations were performed using Microsoft Excel.
Peak areas: 1 decimal place
Slope: 5 significant figures
Correlation coefficient: 6 decimal places
Concentration: 5 significant figures
Ratio of retention time: 3 decimal places
Content (mg): 5 significant figures
Related substances (%): 2 decimal places
Relative retention time: 2 decimal places
Other values expressed as a percentage: 1 decimal place

### Example 8

### Validation of Primary Particle Size Analysis Method in Mastersizer 2000 of Epinephrine Powder

This validation study was performed to ensure the suitability of primary particle size analysis method of epinephrine using Mastersizer 2000, a laser diffraction particle size analyzer.

Materials

Test Articles
Name: (−) Epinephrine
Manufacturer: Sigma-Aldrich. Co. LLC.
Lot number: BCBJ0169V
Amount allocated: 5 vial (5 g)
Storage conditions: Refrigerated and protected from light in a tight container
Storage location: Test Article Refrigerator in the Test Article Depository (acceptable range: 2° C. to 8° C.)
Handling instructions: A mask, a cap, gloves, and safety glasses were worn.
Handling of remaining: All remaining test articles were discarded by the completion of the experiment.

Standard Substance

| Name | Batch No. | Manufacturer |
|---|---|---|
| Malvern Quality Audit Standard | QAS3002 | Malvern Instruments Limited |

Test Equipment

| Equipment | Model | Manufacturer |
|---|---|---|
| Optical Bench | Mastersizer 2000 | Malvern Instruments |
| Dispersion Unit | Scirocco 2000 | Limited |
| Data Management System | Malvern Software | |
| Wet Sample Dispersion Unit | Hyrdo 2000SM | |

Primary Particle Size Analyses

Dry Dispersion Method

| Test frequency: | one time per each pressure | |
|---|---|---|
| Amount of | 150 to 200 mg/run | |
| epinephrine per | | |
| run: | | |
| Sample tray: | General purpose sample tray | |
| Mastersizer | Sampler Selection: | Scirocco |
| 2000 software | Material: | |
| settings: | Material: | Default (1.520) |
| | Absorption: | 0.1 |
| | Dispersant: | Air (1.000) |
| | Result Calculation: | |
| | Analysis Model: | General Purpose |
| | Sensitivity: | Normal |
| | Particle Shape: | Irregular |
| | Report/Saving: | Always save results |
| | Measurement: | |
| | Measurement Time: | 5 seconds |
| | Measurement Snap: | 5,000 |
| | Background Time: | 5 seconds |
| | Measurement Snap: | 5,000 |

| | |
|---|---|
| Dispersion air | Conduct primary particle size analysis on dispersion air pressure of 0.5, 1.0, 2.0, |
| pressure: | 3.0 and 4.0 bar. |
| Feed rate: | 50% |
| Width of | Approximately 4 to 5 mm |
| sample gate: | |
| Obscuration | 1 to 8% |
| limits: | |
| Test method: | Instrument/Software Set-up |
| | Turn instrument on. Make sure the Sirocco dry powder feeder attachment, hose |
| | and flow cell is connected properly. Make sure the vacuum and compressed air |
| | cylinder is connected. Turn on the Scirocco module. Allow laser to warm up for |
| | 30 minutes. |
| | Sample Analysis |
| | Adjust the sample gates to measure approximately 4 to 5 mm in width. Transfer a |
| | representative portion of the test material into the dry powder feeder hopper. Enter |
| | sample ID information. The system automatically aligns the laser and measures |
| | background (for 5,000 snaps or 5 seconds). Hit the OK button to start the analysis. |
| | Result is automatically saved. Change the dispersion air pressure and repeat the |
| | measurement. |
| Parameters | Particle size (μm) |
| | Volume under 90%: d(0.9) |
| | Volume under 50%: d(0.5) |
| | Volume under 10%: d(0.1) |

Wet Dispersion Method

| Test frequency: | 1 measurement/run, 1 run. | |
|---|---|---|
| Amount of epinephrine per run: | 10 mg/run | |
| astersizer 2000 software | Sampler Selection: | Hyrdo 2000SM |
| settings: | Material: | |
| | Material: | Default (1.520) |
| | Dispersant: | Water |
| | Result Calculation: | |
| | Analysis Model: | General Purpose |
| | Sensitivity: | Normal |
| | Particle Shape: | Irregular |
| | Report/Saving: | Always save results |
| | Measurement: | |
| | Measurement Time: | 5 seconds |
| | Measurement Snap: | 5,000 |
| | Background Time: | 5 seconds |
| | Background Snap: | 5,000 |
| | Measurement Cycles: | |
| | Cycles: | 1 times |
| | Measurement Interval: | None |

| | |
|---|---|
| Dispersion unit controller: | 2000 ± 200 rpm |
| Obscuration limits: | 1 to 8% |
| Test method: | Instrument/Software Set-up |
| | Turn on the Mastersizer 2000. Allow laser to warm up for 30 |
| | minutes. Connect the Wet Sample Dispersion Unit to Mastersizer |
| | 2000. Fill the Wet Sample Dispersion Unit with dispersant. |
| | Sample Analysis |
| | Measure the background. Add test article, one droplet at a time, |
| | until obscuration measurement is within 1-8%. Start the particle |
| | size measurement. Results of the measurement were automatically |
| | saved. Report the result measurement. |
| Parameters | Particle size (μm) |
| | Volume under 90%: d(0.9) |
| | Volume under 50%: d(0.5) |
| | Volume under 10%: d(0.1) |

Data Handling
1. Using Mastersizer 2000 software, combine particle size distribution data obtained for each dispersion pressure.
2. If necessary, conduct particle size analysis using Wet Dispersion Method.
3. Choose the optimum dispersion pressure.

Rounding of Figures
Dispersion air pressure (bar): 1 decimal place

Results

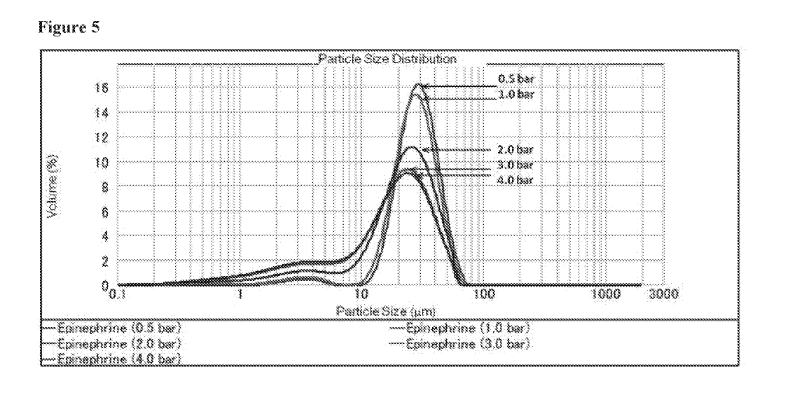

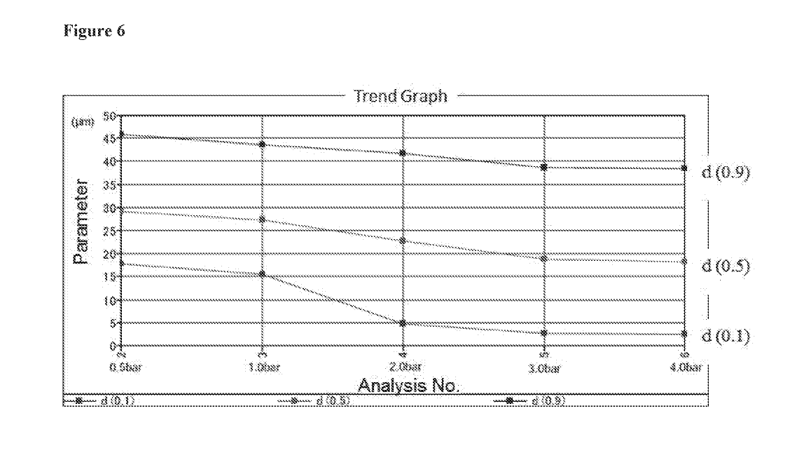

This validation study was performed to ensure the suitability of primary particle size analysis method of epinephrine using Mastersizer 2000, a laser diffraction particle size analyzer. Primary particle size distribution on each dispersion air pressure (also illustrated in FIG. 5; trend data are shown in FIG. 6).

| Dispersion | Volume | Volume | Volume |
|---|---|---|---|
| air pressure | under 90% | under 50% | under 10% |
| (bar) | (μm) | (μm) | (μm) |
| 0.5 | 45.9 | 29.1 | 17.8 |
| 1.0 | 43.6 | 27.2 | 15.5 |
| 2.0 | 41.8 | 22.6 | 4.8 |
| 3.0 | 38.6 | 18.8 | 2.7 |
| 4.0 | 38.3 | 18.2 | 2.4 |

In comparison with Dry Dispersion Method, primary particle size analysis using Wet Dispersion Method was conducted.

Primary particle size distribution using Wet Dispersion Method

| Volume under | Volume under | Volume under |
|---|---|---|
| 90% (μm) | 50% (μm) | 10% (μm) |
| 50.7 | 31.2 | 18.1 |

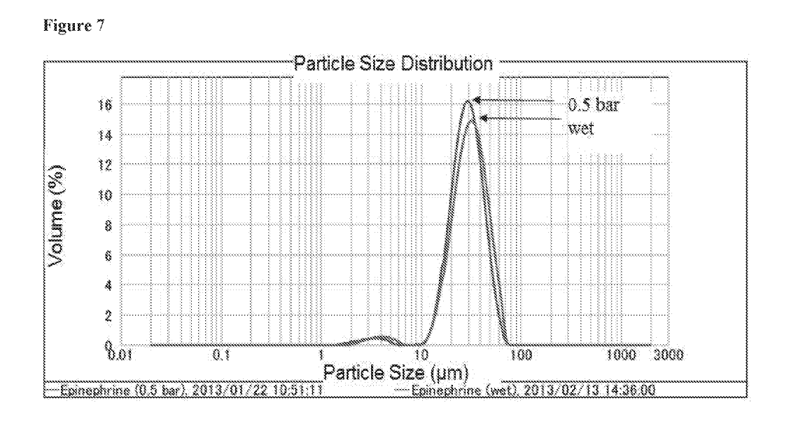

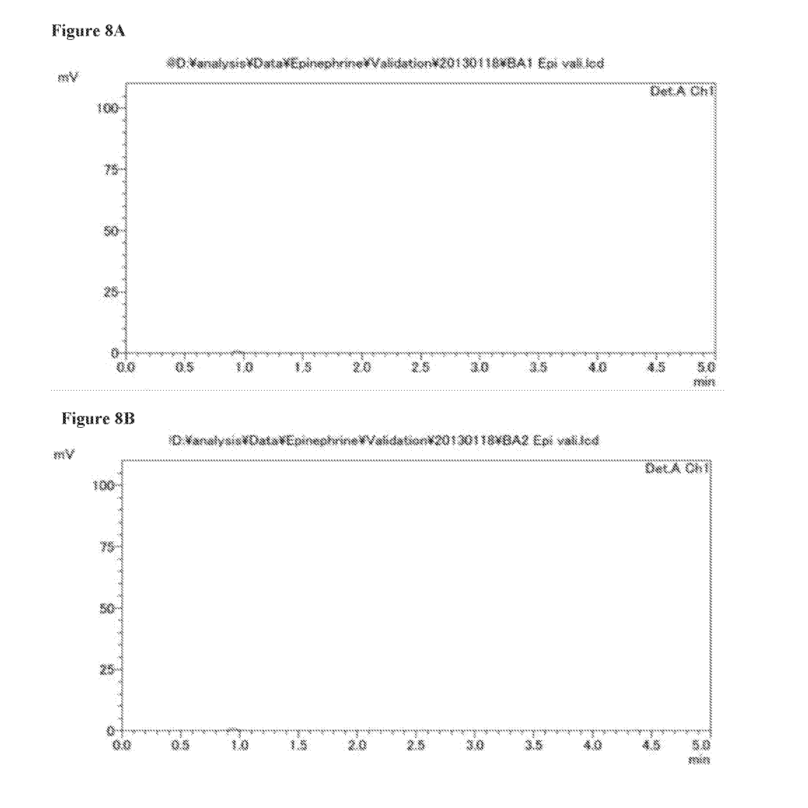

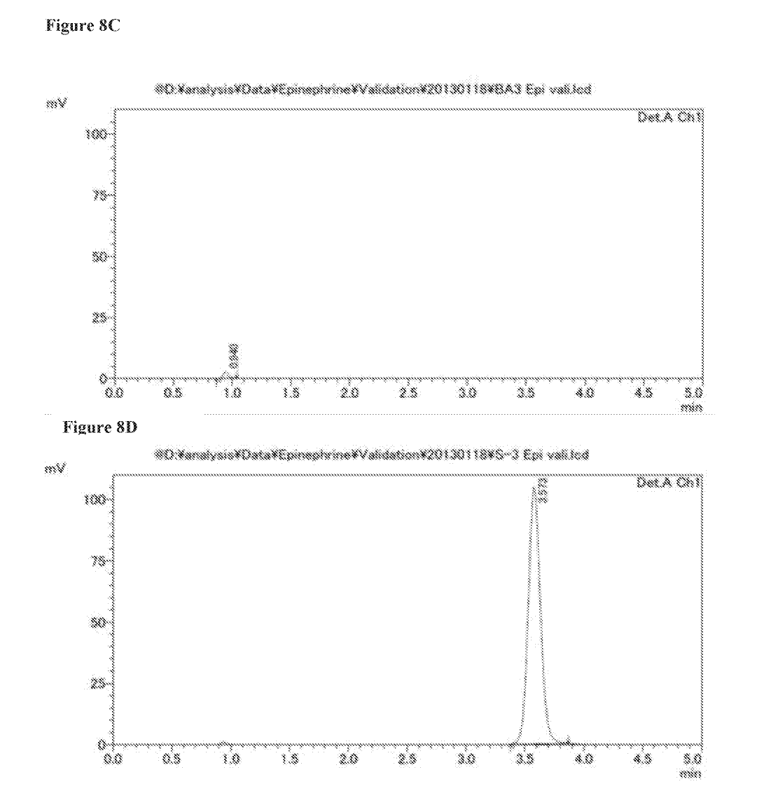

FIG. 7 shows comparison of primary particle size distribution between Dry Dispersion Method (0.5 bar) and Wet Dispersion Method.

Particle size distribution obtained using Dry Dispersion Method at 0.5 bar was comparable to that obtained using Wet Dispersion Method, and particle size distribution using Dry Dispersion Method generally decreased in size at pressures higher than 1.0 bar. It is determined that epinephrine particles were dispersed to its primary particles when analyzed using Dry Dispersion Method at 0.5 bar and was milled to smaller particles at pressures higher than 1.0 bar. Therefore, 0.5 bar was chosen as the optimal pressure when analyzing this epinephrine powder using Dry Dispersion Method.

### Example 9

### Validation of an HPLC/UV Method for Analysis of Epinephrine in Preparations

This validation study was performed to ensure the suitability of a reversed-phase HPLC-UV analytical method for assay of epinephrine in preparations.

Test Article

(−) Epinephrine, from Sigma-Aldrich. Co. LLC.
Storage conditions: Refrigerated and protected from light in a tight container
Storage location: Test Article Refrigerator in the Test Article Depository (acceptable range: 2° C. to 8° C.)
Handling instructions: A mask, a cap, safety glasses, gloves, and a disposable lab coat were worn.

Experiment I

Matrix Component

| Name | Grade |
|---|---|
| Excipient 1 | NF |
| Excipient 2 | NF |
| Excipient 3 | NF |
| Lactose(Raspitose SV003) | NF |
| (DMV INTERNATIONAL) | |

Materials

Reagents

| Name | Grade | Manufacturer |
|---|---|---|
| Monobasic sodium | Special grade | Wako Pure Chemical Industries, |
| phosphate | | Ltd. |
| Sodium 1- | Ion-pair | Wako Pure Chemical Industries, |
| octanesulfonate | chromatography | Ltd. |
| | grade | |
| Disodium edetate | JP | Wako Pure Chemical Industries, |
| hydrate | | Ltd. |
| Phosphoric acid | Special grade | Wako Pure Chemical Industries, Ltd |
| Methanol | HPLC grade | Wako Pure Chemical Industries, Ltd |
| Sodium chloride | Special grade | Wako Pure Chemical Industries, Ltd |
| Sodium metabisulfite | Special grade | Wako Pure Chemical Industries, Ltd |
| 1 mol/1 hydrochloric | Special grade | Wako Pure Chemical Industries, Ltd |
| acid | | |

| | |
|---|---|
| Ultrapure water | Water purified using the Milli-Q system |

Other Materials

| Name | Model | Manufacturer |
|---|---|---|
| Syringe | DISMIC-3JP, DISMIC-13JP, | Toyo Roshi Kaisha, |
| filter | DISMIC-25JP (PTFE, 0.50 μm) | Ltd. |

Measurement Equipment

| Name | Model | Manufacturer |
|---|---|---|
| HPLC system | Shimadzu 20A | Shimadzu Corp. |
| UV detector | Shimadzu SPD-20A | Shimadzu Corp. |
| Data management system | LCsolution | Shimadzu Corp. |

Preparation of Mobile Phases and Reagents

Preparation of Mobile Phase

To 1 L of 0.05 M monobasic sodium phosphate was added about 519 mg of sodium 1-octanesulfonate and about 50 mg of disodium edetate hydrate, and the solution was mixed. The pH was adjusted by the dropwise addition of phosphoric acid to a pH of 3.8. 85 volumes of this solution were mixed with 15 volumes of methanol. The solution was degassed for 10 minutes under reduced pressure and then sonicated for 5 minutes.

Preparation of Diluent

### Mobile Phase.

Preparation of Specificity Solution

Intranasal Powder Formulation
1. The component of BA was accurately weighed, and transferred to a volumetric flask.
2. The diluent was adjusted to about 80% of volumetric flask and sonicated for 10 minutes before diluting with the remaining diluent to target volume.
3. An aliquot was filtered through a syringe filter for analysis. Specificity solutions were prepared (n=1).

| | | Target | | Volumetric |
|---|---|---|---|---|
| Solution | Matrix Component | Weight | | flask |
| No. | [acceptable range] | (mg) | Diluent | (mL) |
| BA1 | Excipient 1 [±1%] | 136.0 | mobile phase | 100 |
| | Excipient 2 [±1%] | 16.0 | | |
| | Excipient 3 [±10%] | 2.0 | | |
| BA2 | Lactose[±1%] | 148.0 | mobile phase | 200 |

BA1 = Equivalent to the total amount of carrier contained in 8 capsules of 0.75 mg epinephrine/20 mg intranasal powder formulation.
BA2 = Equivalent to the total amount of carrier contained in 8 capsules of 1.5 mg epinephrine/20 mg intranasal powder formulation (Lactose carrier).

IM solution
1. The component of BA was accurately weighed, and transferred to a volumetric flask.
2. The diluent was adjusted to about 50% of volumetric flask and 1 mol/L hydrochloric acid to adjust pH 2.2-5.0.
3. Sonicated for 10 minutes before diluting with the remaining diluent to target volume.

| Interim | | Target | | Volumetric |
|---|---|---|---|---|
| Solution | Matrix Component | Weight | | flask |
| No. | [acceptable range] | (mg) | Diluent | (mL) |
| ba3 | Sodium chloride [±1%] | 90.0 | Water for | 15 |
| | Sodium metabisulfite | 25.0 | injection | |
| | [±1%] | | | |

4. In accordance with the following table, interim sample solution was diluted with the diluent (n=1).

| Sample | Sampled | Amount | | Final volume |
|---|---|---|---|---|
| Solution No. | from | (mL) | Diluent | (mL) |
| BA3 | ba3 | 3.0 | Water for injection | 25 |

5. An aliquot was filtered through a syringe filter for analysis.

Preparation of Standard Solutions

Standard solutions were prepared just before analysis in volumetric flasks.
1. In order to first prepare an original standard solution (SSA), 15 mg of epinephrine was accurately weighed, and transferred to a 50 mL volumetric flask.
2. The diluent was added and sonicated for 10 minutes before diluting with the diluent to volume.
3. SSA was prepared standard solutions at 150%, 125%, 100%, 75% and 50% of the target concentration of 60 μg/mL.
4. In accordance with the following table, predetermined volume of SSA was mixed with the diluent to about 80% of the capacity of the flask.
5. Diluted with the diluent to the final volume. Mixed well.
6. An aliquot was filtered through a syringe filter for analysis.

| Standard | | | | Final | Concen- |
|---|---|---|---|---|---|
| Solution | Sampled | Amount | | volume | tration |
| No. | from | (mL) | Diluent | (mL) | (μg/mL) |
| SSA | — | About 15 mg | mobile phase | 50 | ca. 300 |
| S-5 | SSA | 3.0 | mobile phase | 10 | ca. 90 |
| S-4 | SSA | 2.5 | mobile phase | 10 | ca. 75 |
| S-3 | SSA | 2.0 | mobile phase | 10 | ca. 60 |
| S-2 | SSA | 3.0 | mobile phase | 20 | ca. 45 |
| S-1 | SSA | 1.0 | mobile phase | 10 | ca. 30 |

Preparation of Sample Solutions

Intranasal Powder Formulation
1. The components of T1 and T2 were accurately weighed, and transferred to a volumetric flask.
2. The Diluent was adjusted to about 80% of volumetric flask and sonicated for 10 minutes before diluting with the remaining diluent to target volume.
3. An aliquot was filtered through a syringe filter for analysis. Sample solutions were prepared (n=3) just before analysis (T1-1, T1-2, T1-3, T2-1, T2-2, T2-3, T3-1, T3-2 and T3-3).

| Sample | Component | Target | | Final | Concen- |
|---|---|---|---|---|---|
| Solution | [acceptable | Weight | | Volume | tration |
| No. | range] | (mg) | Diluent | (mL) | (μg/mL) |
| T1 | Epinephrine | 6.0 | mobile | 100 | ca. 60 |
| | [±1%] | | phase | | |
| | Excipient 1 [±1%] | 136.0 | | | |
| | Excipient 2 [±1%] | 16.0 | | | |
| | Excipient 3 | 2.0 | | | |
| | [±10%] | | | | |
| T2 | Epinephrine | 12.0 | mobile | 200 | ca. 60 |
| | [±1%] | | phase | | |
| | Excipient 1 [±1%] | 130 | | | |
| | Excipient 2 [±1%] | 16.0 | | | |
| | Excipient 3 | 2.0 | | | |
| | [±10%] | | | | |
| T3 | Epinephrine | 12.0 | mobile | 200 | ca. 60 |
| | [±1%] | | phase | | |
| | Lactose[±1%] | 148.0 | | | |

T1: Equivalent to the contents of 8 capsules of 0.75 mg epinephnne/20 mg intranasal powder formulation
T2: Equivalent to the contents of 8 capsules of 1.5 mg epinephrine/20 mg intranasal powder formulation
T3: Equivalent to the contents of 8 capsules of 1.5 mg epinephrine/20 mg intranasal powder formulation (lactose carrier).

1M solution
1. The component of t4 and t5 was accurately weighed, and transferred to a volumetric flask.
2. The diluent was adjusted to about 50% of volumetric flask and hydrochloric acid to adjust pH 2.2-5.0.
3. Sonicated for 10 minutes before diluting with the remaining diluent to target volume.

| Interim | | | | | |
|---|---|---|---|---|---|
| Sample | Component | Target | | Final | Concen- |
| Solution | [acceptable | Weight | | Volume | tration |
| No | range] | (mg) | Diluent | (mL) | (mg/mL) |
| t4 | Epinephrine [±1%] | 7.5 | Water for | 15 | 0.5 |
| | Sodium chloride | 90.0 | injection | | |
| | [±1%] | | | | |
| | Sodium | 25.0 | | | |
| | metabisulfite [±1%] | | | | |
| t5 | Epinephrine [±1%] | 15 | | 15 | 1.0 |
| | Sodium chloride | 90.0 | | | |
| | [±1%] | | | | |
| | Sodium | 25.0 | | | |
| | metabisulfite [±1%] | | | | |

4. In accordance with the following table, interim sample solution was diluted with the diluent. (T4-1, T4-2, T4-3, T5-1, T5-2 and T5-3)

| Sample | Sampled | Amount | | Final volume |
|---|---|---|---|---|
| Solution No. | from | (mL) | Diluent | (mL) |
| T4 | t4 | 3.0 | Water for | 25 |
| T5 | t5 | 3.0 | injection | 50 |

5. An aliquot was filtered through a syringe filter for analysis.

Analysis Method

HPLC Conditions
HPLC system: Shimadzu 20A
Detector: Ultraviolet absorption photometer (Shimadzu SPD-20A) Wavelength: 280 nm
Data management system: LCsolution
Analysis Column: Zorbax SB-C8 (4.6 mm i.d.×150 mm, 5 μm) Agilent Technologies
Column temperature: Set at 25° C.
Sample temperature: Set at 5° C.
Flow rate: 2.0 mL/min
Injection volume: 20 μL

System Repeatability

Five replicate injections of standard solution S-3 were performed at the beginning of analysis, one injection every 10 to 15 injections and one injection at the end of analysis. LCsolution was used to calculate the epinephrine peak areas and the relative standard deviation (RSD, %).

### Acceptance Criteria:

An RSD for the first five injections not exceeding 2.0%
An RSD for all injections not exceeding 3.0%

Validation Items

Specificity

Specificity solutions and standard solution S-3 each were injected once onto the HPLC.

Acceptance criterion:
No interfering peak at the retention times of epinephrine should be observed from the corresponding specificity solution.

Linearity

The standard solutions (S-1 to S-5) were each be injected (n=1) onto the HPLC. Using LCsolution, linear regression curves from S-1 to S-5 were constructed from the peak areas (Y) and epinephrine concentrations (X). The correlation coefficient (r), slope, Y-intercept and relative deviations from the regression line at each concentration were obtained.

### Acceptance Criteria:

Correlation coefficients (r) no less than 0.99
Relative deviations: From S-1 to S-5 within ±5.0%

Relative deviation (%)=[(Peak area Y−intercept)/slope−theoretical concentration]/theoretical concentration×100

Accuracy and Repeatability

The sample solution (n=1) was injected onto the HPLC. Using LCsolution, the peak areas were obtained from which the assay (%) was calculated as follows.

$$Assay ( \% ) = \frac{\begin{matrix}Epinephrine concentration in \\ sample solution ( μg \text{/} mL ) \end{matrix}}{Actual amount weighed ( mg )} \times \frac{\begin{matrix}Dilution \\ Factor \end{matrix}}{1000} \times 100$$

The mean and relative standard deviation (RSD, %) for each level was calculated.

RSD (%)=SD/Mean×100

### Acceptance Criteria:

Mean assay values within 100.0%±5.0% at each level (accuracy)
An RSD not exceeding 5.0% at each level (repeatability)

Solution Stability

Standard solutions for linearity (S-3) and sample solution (T1-1, T2-1, T3-1, T4-1 and T5-1) were analyzed in duplicate after system repeatability and after storage in the autosampler for 24 hours. The mean peak areas after the system reproducibility were regarded as the initial values.

### Acceptance Criterion:

Mean peak areas after storage within 100.0%±5.0% of the initial mean peak areas

Data Handling

The slope, correlation coefficient, sample solution concentrations and system reproducibility were obtained using the data management system. Subsequent calculations were performed using Microsoft Excel.
Peak areas: 1 decimal place
Slope: 5 significant figures
Correlation coefficient: 6 decimal places
Concentration: 5 significant figures
Values expressed as a percentage: 1 decimal place

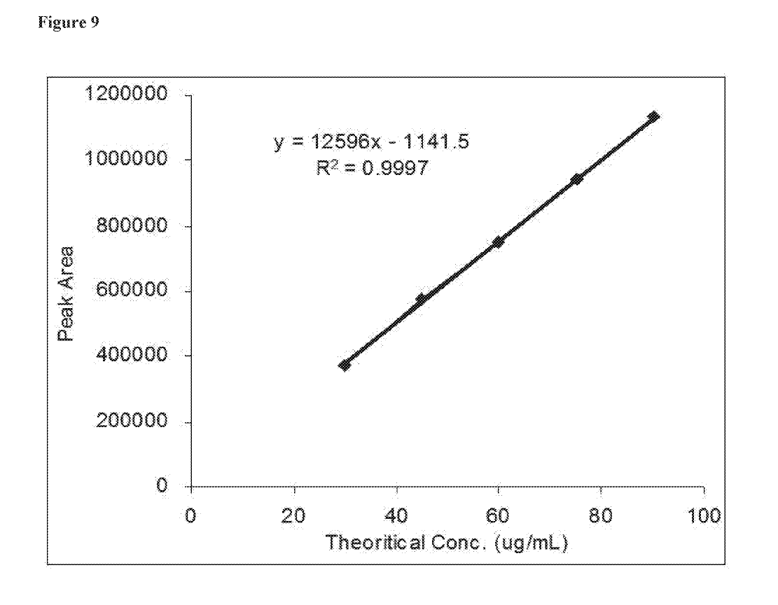

Results of Experiment I
1) Specificity (FIGS. 5A-5D): No interfering peaks at the retention time of the peak detected from the standard solution were observed from the corresponding specificity solution.
2) Linearity (FIG. 9, Table 7): A straight line within the concentration range 30 to 90 μg/mL confirmed linearity.
3) Accuracy and repeatability (Table 8): The mean assay values and relative standard deviation (RSD) values met the acceptance criteria confirming accuracy and repeatability.
4) Solution stability (Table 9): Analysis solutions were confirmed to be stable in the autosampler for 24 hours.

From the above, it was concluded that this reversed-phase HPLC-UV analytical method is suitable for assay and related substances determinations of Epinephrine in intranasal powder formulations.

TABLE 7
Linearity

| | Nominal | | Calculated | Relative |
|---|---|---|---|---|
| Sample | concentration | Peak | concentration | deviation |
| name | (μg/mL) | area | (μg/mL) | (%) |
| S-1 | 90 | 1133755.9 | 90.099 | −0.1 |
| S-2 | 75 | 9411224.3 | 74.806 | 0.3 |
| S-3 | 60 | 751375.6 | 59.742 | 0.4 |
| S-4 | 45 | 574560.5 | 45.705 | −1.6 |
| S-5 | 30 | 372325.5 | 29.649 | 1.2 |

| | |
|---|---|
| Correlation coefficient | 0.999674 |
| Slope | 12596 |
| Y-intercept | −1141.5 |

### Acceptance Criteria:

Correlation coefficients (r) no less than 0.99
Relative deviations: From S-1 to S-5 within ±5.0%

TABLE 8
Accuracy and repeatability

| | | Determined | Actual | | | Mean | | |
|---|---|---|---|---|---|---|---|---|
| Sample | Peak area | Epinephrine | amount | Dilution | Assay | assay | | |
| name | Mean | conc. (μg/mL) | (mg) | factor | (%) | (%) | SD | RSD (%) |
| T1-1 | 756132.0 | 60.119 | 6.02 | 100 | 99.9 | 100.3 | 0.5 | 0.5 |
| T1-2 | 754832.5 | 60.016 | 5.99 | | 100.2 | | | |
| T1-3 | 762967.5 | 60.662 | 6.02 | | 100.8 | | | |
| T2-1 | 759909.2 | 60.419 | 12.00 | 200 | 100.7 | 101.1 | 1.1 | 1.1 |
| T2-2 | 770749.6 | 61.280 | 11.98 | | 102.3 | | | |
| T2-3 | 757204.2 | 60.204 | 12.02 | | 100.2 | | | |
| T3-1 | 754099.0 | 59.958 | 11.97 | 200 | 100.2 | 100.4 | 0.4 | 0.4 |
| T3-2 | 754617.9 | 59.999 | 11.98 | | 100.2 | | | |
| T3-3 | 759885.2 | 60.417 | 11.98 | | 100.9 | | | |
| T4-1 | 772246.3 | 61.399 | 7.49 | 125 | 102.5 | 102.5 | 0.0 | 0.0 |
| T4-2 | 772319.7 | 61.405 | 7.49 | | 102.5 | | | |
| T4-3 | 772724.9 | 61.437 | 7.49 | | 102.5 | | | |
| T5-1 | 756152.0 | 60.121 | 14.99 | 250 | 100.3 | 100.5 | 0.9 | 0.9 |
| T5-2 | 751706.0 | 59.768 | 14.99 | | 99.7 | | | |
| T5-3 | 765050.1 | 60.827 | 14.99 | | 101.4 | | | |

### Acceptance Criteria:

Mean assay values within 100.0%±5.0% at each level (accuracy)
An RSD not exceeding 5.0% at each level (repeatability)

TABLE 9
Solution stability

| | | | | | |
|---|---|---|---|---|---|
| | | Peak | Mean | Stability | |
| Sample name | Storage | area | peak area | (%) | Judgment |

Standard solution (S-3)

| | | | | | |
|---|---|---|---|---|---|
| S3-1 | Initial | 747341.9 | 747334.1 | 100.0 | |
| S3-2 | | 747326.2 | | | |
| S3-1 | After 24 hours | 763699.7 | 763520.5 | 102.2 | Passed |
| S3-2 | (autosampler) | 763341.3 | | | |

Sample solution (T1-1)

| | | | | | |
|---|---|---|---|---|---|
| T1-1(1) | Initial | 750923.7 | 751479.3 | 100.0 | |
| T1-1(2) | | 752034.9 | | | |
| T1-1(1) | After 24 hours | 761685.5 | 761772.7 | 101.4 | Passed |
| T1-1(2) | (autosampler) | 761859.9 | | | |

Sample solution (T2-1)

| | | | | | |
|---|---|---|---|---|---|
| T2-1(1) | Initial | 760459.4 | 759795.6 | 100.0 | |
| T2-1(2) | | 759131.8 | | | |
| T2-1(1) | After 24 hours | 767090.9 | 767252.1 | 101.0 | Passed |
| T2-1(2) | (autosampler) | 767413.3 | | | |

Sample solution (T3-1)

| | | | | | |
|---|---|---|---|---|---|
| T3-1(1) | Initial | 749588.4 | 749554.6 | 100.0 | |
| T3-1(2) | | 749520.7 | | | |
| T3-1(1) | After 24 hours | 765792.2 | 766789.2 | 102.3 | Passed |
| T3-1(2) | (autosampler) | 767786.2 | | | |

Sample solution (T4-1)

| | | | | | |
|---|---|---|---|---|---|
| T4-1(1) | Initial | 762376.2 | 762734.5 | 100.0 | |
| T4-1(2) | | 763092.7 | | | |
| T4-1(1) | After 24 hours | 767786.2 | 767650.6 | 100.6 | Passed |
| T4-1(2) | (autosampler) | 767515.0 | | | |

Sample solution (T5-1)

| | | | | | |
|---|---|---|---|---|---|
| T5-1(1) | Initial | 748559.1 | 748363.6 | 100.0 | |
| T5-1(2) | | 748168.0 | | | |
| T5-1(1) | After 24 hours | 763825.3 | 763894 | 102.1 | Passed |
| T5-1(2) | (autosampler) | 763962.7 | | | |

### Acceptance Criteria:

Mean peak areas after storage within 100.0%±5.0% of the initial mean peak areas

Experiment II

Matrix Component

| Name | Grade |
|---|---|
| Excipient 1 | NF |
| Excipient 2 | NF |
| Excipient 3 | NF |

Materials

Reagents

| Name | Grade | Manufacturer |
|---|---|---|
| Monobasic sodium | Special grade | Wako Pure Chemical Industries, |
| phosphate | | Ltd. |
| Sodium 1- | Ion-pair | Wako Pure Chemical Industries, |
| octanesulfonate | chromatography | Ltd. |
| | grade | |
| Disodium edetate | JP | Wako Pure Chemical Industries, |
| hydrate | | Ltd. |
| Phosphoric acid | Special grade | Wako Pure Chemical Industries, Ltd |
| Methanol | HPLC grade | Wako Pure Chemical Industries, Ltd |

| | |
|---|---|
| Ultrapure water | Water purified using the Milli-Q system |

Other Materials

| Name | Model | Manufacturer |
|---|---|---|
| Syringe | DISMIC-3JP, DISMIC-13JP, | Toyo Roshi |
| filter | DISMIC-25JP (PTFE, 0.50 μm) | Kaisha, Ltd. |

Measurement Equipment

| Name | Model | Manufacturer |
|---|---|---|
| HPLC system | Shimadzu 20A | Shimadzu Corp. |
| UV detector | Shimadzu SPD-20A | Shimadzu Corp. |
| Data management system | LCsolution | Shimadzu Corp. |

Preparation of Mobile Phases and Reagents

Preparation of Mobile Phase

To 1 L of 0.05 M monobasic sodium phosphate was added about 519 mg of sodium 1-octanesulfonate and about 50 mg of disodium edetate hydrate, and the solution was mixed. The pH was adjusted by the dropwise addition of phosphoric acid to a pH of 3.8. 85 volumes of this solution were mixed with 15 volumes of methanol. The solution was degassed for 10 minutes under reduced pressure and then sonicated for 5 minutes.

Preparation of Diluent

### Mobile Phase.

Preparation of Specificity Solution

### Intranasal Powder Formulation

1. The component of BA was accurately weighed, and transferred to a volumetric flask.
2. The diluent was adjusted to about 80% of volumetric flask and sonicated for 10 minutes before diluting with the remaining diluent to target volume.
3. An aliquot was filtered through a syringe filter for analysis. Specificity solutions were prepared (n=1).

| | Matrix Component | Target | | Volumetric |
|---|---|---|---|---|
| Solution | [acceptable | Weight | | flask |
| No. | range] | (mg) | Diluent | (mL) |
| BA1 | Excipient 1 [±1%] | 82.8 | mobile phase | 100 |
| | Excipient 2 [±1%] | 16.0 | | |
| | Excipient 3 [±10%] | 2.0 | | |

BA1 = Equivalent to the total amount of carrier contained in 8 capsules of 7.5 mg epinephrine/20 mg intranasal powder formulation.

4. In accordance with the following table, interim sample solution was diluted with the diluent (n=1).

| Sample | Sampled | Amount | | Final |
|---|---|---|---|---|
| Solution No. | from | (mL) | Diluent | volume (mL) |
| BA1 | ba1 | 1.0 | mobile phase | 10 |

5. An aliquot was filtered through a syringe filter for analysis.

Preparation of Standard Solutions

Standard solutions were prepared just before analysis in volumetric flasks.
1. In order to first prepare an original standard solution (SSA), 15 mg of epinephrine was accurately weighed, and transferred to a 50 mL volumetric flask.
2. The diluent was added and sonicated for 10 minutes before diluting with the diluent to volume.
3. SSA was prepared standard solutions at 150%, 125%, 100%, 75% and 50% of the target concentration of 60 μg/mL.
4. In accordance with the following table, predetermined volume of SSA was mixed with the diluent to about 80% of the capacity of the flask.
5. Diluted with the diluent to the final volume. Mixed well.
6. An aliquot was filtered through a syringe filter for analysis.

| Standard | | | | Final | |
|---|---|---|---|---|---|
| Solution | Sampled | | | volume | Concentration |
| No. | from | Amount (mL) | Diluent | (mL) | (μg/mL) |
| SSA | — | About 15 mg | mobile phase | 50 | ca. 300 |
| S-5 | SSA | 3.0 | mobile phase | 10 | ca. 90 |
| S-4 | SSA | 2.5 | mobile phase | 10 | ca. 75 |
| S-3 | SSA | 2.0 | mobile phase | 10 | ca. 60 |
| S-2 | SSA | 3.0 | mobile phase | 20 | ca. 45 |
| S-1 | SSA | 1.0 | mobile phase | 10 | ca. 30 |

Preparation of Sample Solutions

### Intranasal Powder Formulation

1. The component of t1 was accurately weighed, and transferred to a volumetric flask.
2. The Diluent was adjusted to about 80% of volumetric flask and sonicated for 10 minutes before diluting with the remaining diluent to target volume.

| Sample | Component | Target | | Final | Concen- |
|---|---|---|---|---|---|
| Solution | [acceptable | Weight | | Volume | tration |
| No. | range] | (mg) | Diluent | (mL) | (μg/mL) |
| t1 | Epinephrine | 60.0 | mobile | 100 | ca. 600 |
| | [±1%] | | phase | | |
| | Excipient 1 [±1%] | 82.8 | | | |
| | Excipient 2 [±1%] | 16.0 | | | |
| | Excipient 3 | 2.0 | | | |
| | [±10%] | | | | |

t1: Equivalent to the contents of 8 capsules of 7.5 mg epinephrine/20 mg intranasal powder formulation

3. In accordance with the following table, interim sample solution was diluted with the diluent.

| Sample | Sampled | Amount | | Final |
|---|---|---|---|---|
| Solution No. | from | (mL) | Diluent | volume (mL) |
| T1 | t1 | 1.0 | mobile phase | 10 |

4. An aliquot was filtered through a syringe filter for analysis. Sample solutions were prepared (n=3) just before analysis (T1-1, T1-2 and T1-3)

Analysis Method

HPLC Conditions
HPLC system: Shimadzu 20A
Detector: Ultraviolet absorption photometer (Shimadzu SPD-20A) Wavelength: 280 nm
Data management system: LCsolution
Analysis Column: Zorbax SB-C8 (4.6 mm i.d.×150 mm, 5 μm) Agilent

Technologies
Column temperature: Set at 25° C.
Sample temperature: Set at 5° C.
Flow rate: 2.0 mL/min
Injection volume: 20 μL

System Repeatability

Five replicate injections of standard solution S-3 were performed at the beginning of analysis, one injection every 10 to 15 injections and one injection at the end of analysis. LCsolution was used to calculate the epinephrine peak areas and the relative standard deviation (RSD, %).

Acceptance criteria:
An RSD for the first five injections not exceeding 2.0%
An RSD for all injections not exceeding 3.0%

Validation Items

Specificity

Specificity solutions and standard solution S-3 were each injected once onto the HPLC. Acceptance criterion:
No interfering peak at the retention times of epinephrine should be observed from the corresponding specificity solution.

Linearity

The standard solutions (S-1 to S-5) were each injected (n=1) onto the HPLC. Using LCsolution, linear regression curves from S-1 to S-5 were constructed from the peak areas (Y) and epinephrine concentrations (X). The correlation coefficient (r), slope, Y-intercept and relative deviations from the regression line at each concentration were obtained.

### Acceptance Criteria:

Correlation coefficients (r) no less than 0.99
Relative deviations: From S-1 to S-5 within ±5.0%

Relative deviation (%)=[(Peak area−Y-intercept)/slope−theoretical concentration]/theoretical concentration×100

Accuracy and Repeatability

The sample solution (n=1) was injected onto the HPLC. Using LCsolution, the peak areas were obtained from which the assay (%) was calculated as follows.

$$Assay ( \% ) = \frac{\begin{matrix}Epinephrine concentration in \\ sample solution ( μg \text{/} mL ) \end{matrix}}{Actual amount weighed ( mg )} \times \frac{\begin{matrix}Dilution \\ Factor \end{matrix}}{1000} \times 100$$

The mean and relative standard deviation (RSD, %) for each level was calculated.

RSD (%)=SD/Mean×100

### Acceptance Criteria:

Mean assay values within 100.0%±5.0% at each level (accuracy)
An RSD not exceeding 5.0% at each level (repeatability)

Solution Stability

Standard solutions for linearity (S-3) and sample solution (T1-1) were analyzed in duplicate after system repeatability and after storage in the autosampler for 24 hours. The mean peak areas after the system reproducibility were regarded as the initial values.

### Acceptance Criterion:

Mean peak areas after storage within 100.0%±5.0% of the initial mean peak areas

Data Handling

The slope, correlation coefficient, sample solution concentrations and system reproducibility were obtained using the data management system. Subsequent calculations were performed using Microsoft Excel.
Peak areas: 1 decimal place
Slope: 5 significant figures
Correlation coefficient: 6 decimal places
Concentration: 5 significant figures
Values expressed as a percentage: 1 decimal place

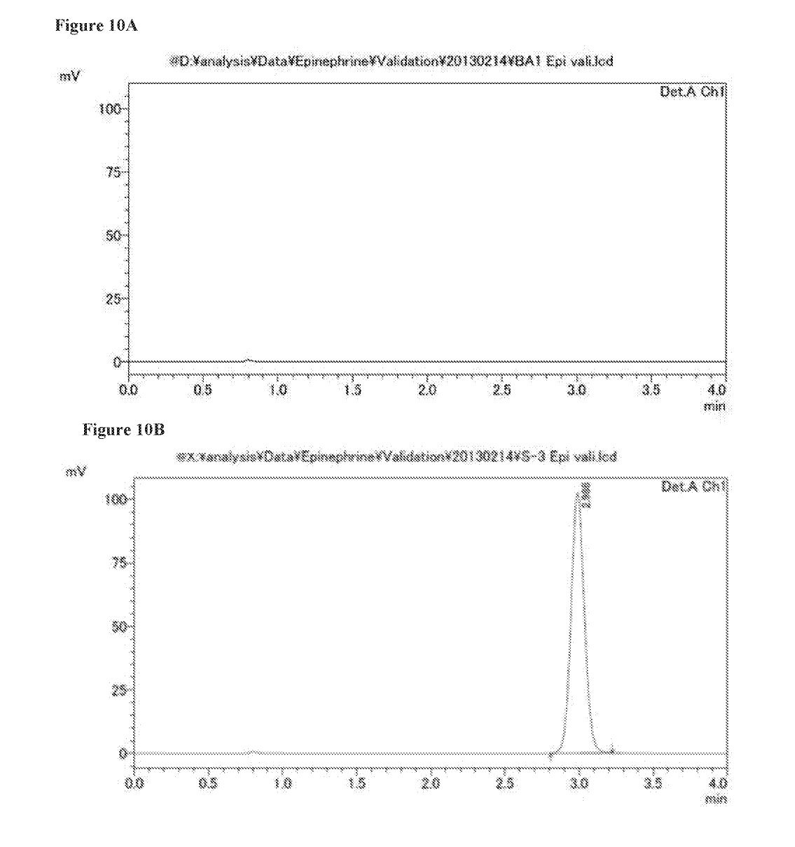

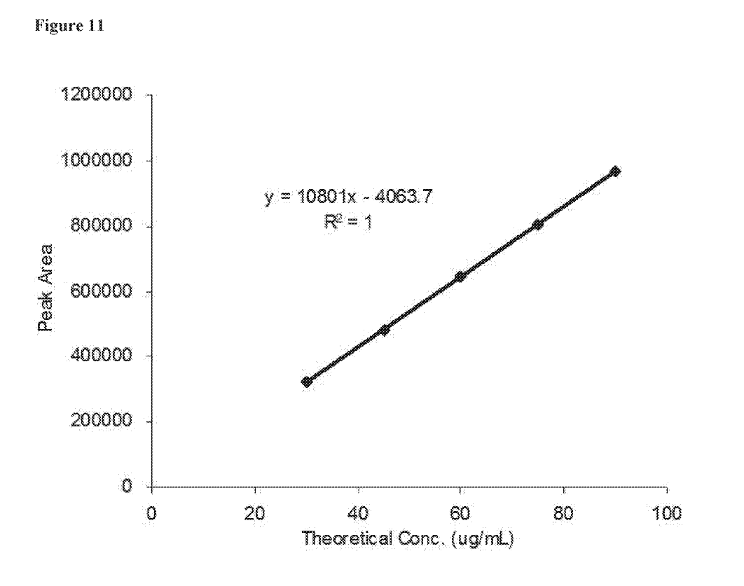

Results of Experiment II
1) Specificity (FIGS. 10A-10B): No interfering peaks at the retention time of the peak detected from the standard solution were observed from the corresponding specificity solution.
2) Linearity (FIG. 11, Table 10): A straight line within the concentration range 30 to 90 μg/mL confirmed linearity.
3) Accuracy and repeatability (Table 11): The mean assay values and relative standard deviation (RSD) values met the acceptance criteria confirming accuracy and repeatability.
4) Solution stability (Table 12): Analysis solutions were confirmed to be stable in the autosampler for 24 hours.

From the above, it was concluded that this reversed-phase HPLC-UV analytical method is suitable for assay determinations of epinephrine in intranasal powder formulations.

TABLE 10
Linearity

| | Nominal | | Calculated | Relative |
|---|---|---|---|---|
| Sample | concentration | Peak | concentration | deviation |
| name | (μg/mL) | area | (μg/mL) | (%) |
| S-1 | 30 | 320756.5 | 30.073 | −0.2 |
| S-2 | 45 | 482024.7 | 45.003 | 0.0 |
| S-3 | 60 | 642399.2 | 59.851 | 0.2 |
| S-4 | 75 | 806001.5 | 74.998 | 0.0 |
| S-5 | 90 | 968857.6 | 90.075 | −0.1 |

| | |
|---|---|
| Correlation coefficient | 0.999985 |
| Slope | 10801 |
| Y-intercept | −4063.7 |

### Acceptance Criteria:

Correlation coefficient (r): No less than 0.99
Relative deviations: From S-1 to S-5 within ±5.0%

TABLE 11
Accuracy and repeatability

| | | Determined | | | | | | |
|---|---|---|---|---|---|---|---|---|
| | | Epinephrine | Actual | | | Mean | | |
| Sample | Peak area | conc. | amount | Dilution | Assay | assay | | |
| name | Mean | (μg/mL) | (mg) | factor | (%) | (%) | SD | RSD (%) |
| T1-1 | 644957.7 | 60.088 | 59.97 | 1000 | 100.2 | 100.1 | 0.1 | 0.1 |
| T1-2 | 643692.8 | 59.971 | 60.00 | | 100.0 | | | |
| T1-3 | 644667.4 | 60.061 | 60.00 | | 100.1 | | | |

### Acceptance Criteria:

Mean assay value within 100.0%±5.0% at each level (accuracy)
An RSD not exceeding 5.0% at each level (repeatability)

TABLE 12
Solution stability

| | | | | | |
|---|---|---|---|---|---|
| Sample | | Peak | Mean | Stability | |
| name | Storage | area | peak area | (%) | Judgment |

Standard solution (S-3)

| | | | | | |
|---|---|---|---|---|---|
| S3-1 | Initial | 642128.7 | 642129.8 | 100.0 | |
| S3-2 | | 642130.8 | | | |
| S3-1 | After 24 hours | 616930.3 | 616696.1 | 96.0 | Passed |
| S3-2 | (autosampler) | 616461.9 | | | |

Sample solution (T1-1)

| | | | | | |
|---|---|---|---|---|---|
| T1-1(1) | Initial | 645597.3 | 644969.1 | 100.0 | |
| T1-1(2) | | 644340.8 | | | |
| T1-1(1) | After 24 hours | 620074.0 | 620178.6 | 96.2 | Passed |
| T1-1(2) | (autosampler) | 620283.1 | | | |

What is claimed is:

1. An intranasal dry powder composition comprising an anti-anaphylactic or anti-anaphylactoid agent.

2. The composition of claim 1, wherein the anti-anaphylactic or anti-anaphylactoid agent is epinephrine or a pharmaceutically acceptable salt thereof.

3. The composition of any one of claims 1-2, wherein the anti-anaphylactic or anti-anaphylactoid agent is about 0.25% to about 50% w/w of the weight of the composition.

4. The composition of claim 3, wherein the anti-anaphylactic or anti-anaphylactoid agent is about 4%, about 7.5%, or about 15% w/w of the weight of the composition.

5. The composition of any one of claims 1-4, wherein a single dose of the anti-anaphylactic or anti-anaphylactoid agent is about 0.01 mg to about 10 mg.

6. The composition of claim 5, wherein a single dose of the anti-anaphylactic or anti-anaphylactoid agent is about 0.75 mg, about 1.5 mg, or about 3.0 mg.

7. The composition of any one of claims 1-6, wherein the dry powder composition when administered to a patient, produces a maximal blood concentration (Cₘₐₓ) of the anti-anaphylactic or anti-anaphylactoid agent at least 2 fold more than the baseline level of the anti-anaphylactic or anti-anaphylactoid agent in the patient.

8. The composition of any one of claims 1-7, wherein the dry powder composition when administered to a patient, reaches a maximal blood concentration (Tₘₐₓ) of the anti-anaphylactic or anti-anaphylactoid agent in less than about 20 minutes after administration.

9. An intranasal dry powder composition comprising a vasoactive agent.

10. The composition of claim 9, wherein the vasoactive agent is epinephrine or a pharmaceutically acceptable salt thereof.

11. The composition of claim 9, wherein the vasoactive agent is vasopressin or a pharmaceutically acceptable salt thereof.

12. The composition of claim 9, wherein the vasoactive agent is atropine or a pharmaceutically acceptable salt thereof.

13. The composition of any one of claims 9-12, wherein the vasoactive agent is about 0.25% to about 50% w/w of the weight of the composition.

14. The composition of claim 13, wherein the vasoactive agent is about 4%, about 7.5%, or about 15% w/w of the weight of the composition.

15. The composition of any one of claims 9-14, wherein a single dose of the vasoactive agent is about 0.01 mg to about 10 mg.

16. The composition of claim 15, wherein a single dose of the vasoactive agent is about 0.75 mg, about 1.5 mg, or about 3.0 mg.

17. The composition of any one of claims 9-16, wherein the dry powder composition when administered to a patient, produces a maximal blood concentration (Cₘₐₓ) of the vasoactive agent at least 2 fold more than the baseline level of the vasoactive agent in the patient.

18. The composition of any one of claims 9-17, wherein the dry powder composition when administered to a patient, reaches a maximal blood concentration (Tₘₐₓ) of the vasoactive agent in less than about 20 minutes after administration.

19. The composition of any one of claims 1-18, wherein the dry powder composition further comprises a vasodilator.

20. The composition of claim 19, wherein the vasodilator is phentolamine or a pharmaceutically acceptable salt thereof.

21. The composition of any one of claims 19-20, wherein the amount of the vasodilator is about 0.005% to about 50% w/w of the weight of the composition.

22. The composition of claim 21, wherein the amount of the vasodilator is about 2.5% w/w of the weight of the composition.

23. The composition of any one of claims 19-22, wherein a single dose of the vasodilator is about 0.01 mg to about 10 mg.

24. The composition of claim 23, wherein a single dose of the vasodilator is about 0.5 mg or about 1.0 mg.

25. The composition of any one of claims 1-24 wherein the dry powder composition further comprises a pharmaceutically acceptable carrier.

26. The composition of claim 25, wherein the carrier further comprises of a first cellulose.

27. The composition of claim 26, wherein the carrier further comprises a second cellulose.

28. The composition of any one of claims 26-27, wherein the carrier further comprises a starch.

29. The composition of any one of claims 26-28, wherein the first cellulose has an average particle diameter of about 100 μm or less.

30. The composition of claim 29, wherein the first cellulose has an average particle diameter of about 30 μm or less.

31. The composition of any one of claims 27-30, wherein the second cellulose, the starch, or the second cellulose and starch each individually has an average particle diameter of about 30 to about 100 μm.

32. The composition of any one of claims 29-31, wherein average particle diameter is determined using a laser-diffraction particle size distribution analyzer.

33. The composition of any one of claims 29-31, wherein average particle diameter is determined using sieve sorting.

34. The composition of any one of claims 26-33, wherein the first cellulose is a crystalline cellulose.

35. The composition of claim 34, wherein the first cellulose is a microcrystalline cellulose.

36. The composition of any one of claims 25-35, wherein the carrier further comprises an excipient.

37. The composition of claim 36, wherein the excipient is about 0.5% to about 5% w/w of the weight of the composition.

38. The composition of any one of claims 25-37, wherein the carrier further comprises an anticaking agent.

39. The composition of claim 38, wherein the anticaking agent is a tribasic calcium phosphate.

40. The composition of any one of claims 1-39, wherein the dry powder composition further comprises a COMT inhibitor.

41. The composition of claim 40, wherein the COMT inhibitor further comprises a reversible COMT inhibitor.

42. The composition of claim 41, wherein the reversible COMT inhibitor is entacapone or a pharmaceutically acceptable salt thereof.

43. A kit comprising:
(a) a dose of the intranasal dry powder composition in any one of claims 1-42;
(b) instructions reciting when the dry powder composition in (a) is to be administered to a subject.

44. The kit of claim 43, further comprising an intranasal delivery apparatus for dispensing the dry powder composition.

45. The kit of claim 44, wherein when activated, the apparatus delivers a therapeutically acceptable amount of the dry powder composition.

46. The kit of claim 45, wherein the dry powder composition is delivered intranasally.

47. The kit of any one of claims 43-46, wherein the apparatus further comprises a reservoir that holds the dry powder composition.

48. The kit of any one of claims 44-47, wherein the apparatus is disposable.

49. The kit of any one of claims 44-47, wherein the apparatus is reusable or recyclable.

50. The kit of any one of claims 43-49, wherein the dry powder composition is a multi-unit package.

51. The kit of claim 50, wherein the package comprises multiple reservoirs, wherein each reservoir contains a single dose of the dry powder composition.

52. The kit of claim 50, wherein the package comprises one apparatus and multiple reservoirs, wherein each reservoir contains a single dose of the dry powder composition.

53. The kit of claim 50, wherein the package comprises multiple apparatuses, wherein each apparatus contains one reservoir which contains a single dose of the dry powder composition.

54. The kit of any one of claims 50-53, wherein the package allows for easy and quick visual verification of units used.

55. The kit of claim 54, wherein the package is labeled for easy and quick visual verification of units used.

56. The kit of claim 55, wherein the package is color labeled for easy and quick visual verification of units used.

57. The kit of any one of claims 43-56, further comprising an automated external defibrillator (AED) system.

58. The kit of claim 57, wherein the instructions further recite how to operate the automated external defibrillator (AED) system.

59. The kit of claim 58, wherein the instructions are pre-loaded on the automated external defibrillator (AED) system.

60. The kit of any one of claims 57-59, wherein the automated external defibrillator (AED) system contains a self-contained power source.

61. The kit of claim 60, wherein the self-contained power source is a battery.

62. The kit of claim 61, wherein the battery is rechargeable.

63. A method comprising treating a patient by intranasally administrating the dry powder composition in any one of claims 1-42.

64. A method comprising treating a patient by using the kit in any one of claims 43-62.

65. The method of claim 63 or 64, wherein the patient has minimal or no cardiac activity.

66. The method of claim 63 or 64, wherein the patient has low blood pressure.

67. The method of claim 63 or 64, wherein the patient has hypotension.

68. The method of claim 63 or 64, wherein the patient is experiencing hypotensive shock.

69. The method of claim 68, wherein the hypotensive shock is secondary to causes comprising trauma.

70. The method of claim 68, wherein the hypotensive shock is secondary to causes comprising hypovolemia.

71. The method of claim 68, wherein the hypotensive shock is secondary to causes comprising bradycardia.

72. The method of claim 68, wherein the hypotensive shock is secondary to causes comprising septic shock.

73. The method of any one of claims 63-72, wherein the intranasal dry powder composition is sufficient to increase the arterial pressure in the patient.

74. The method of claim 73, wherein the intranasal dry powder composition is sufficient to increase the arterial pressure in the patient within 10 minutes after administration.

75. The method of any one of claims 63-72, wherein the intranasal dry powder composition is sufficient to increase the mean arterial pressure in the patient.

76. The method of claim 75, wherein the intranasal dry powder composition is sufficient to increase the mean arterial pressure in the patient within 10 minutes after administration.

77. The method of any one of claims 63-72, wherein the intranasal dry powder composition is sufficient to increase coronary perfusion pressure in the patient.

78. The method of claim 77, wherein the intranasal dry powder composition is sufficient to increase coronary perfusion pressure in the patient within 10 minutes after administration.

79. The method of any one of claims 63-72, wherein the intranasal dry powder composition is sufficient to resume a spontaneous circulation in the patient.

80. The method of claim 79, wherein the intranasal dry powder composition is sufficient to resume a spontaneous circulation in the patient within 10 minutes after administration.

81. The method of any one of claims 63-80, further comprising at least one of (a), (b), or (a) and (b):
(a) initiating cardiopulmonary resuscitation (CPR);
(b) using an automated external defibrillator (AED).

82. The method of claim 81, wherein the intranasal dry powder composition is administered if (a) or (b) fails to increase the arterial pressure in the patient.

83. The method of claim 82, wherein the intranasal dry powder composition is administered if (a) or (b) fails to increase the arterial pressure in the patient within 10 minutes after administration.

84. The method of claim 81, wherein the intranasal dry powder composition is administered if (a) or (b) fails to increase the mean arterial pressure in the patient.

85. The method of claim 84, wherein the intranasal dry powder composition is administered if (a) or (b) fails to increase the mean arterial pressure in the patient within 10 minutes after administration.

86. The method of claim 81, wherein the intranasal dry powder composition is administered if (a) or (b) fails to increase coronary perfusion pressure in the patient.

87. The method of claim 86, wherein the intranasal dry powder composition is administered if (a) or (b) fails to increase coronary perfusion pressure in the patient within 10 minutes after administration.

88. The method of claim 81, wherein the intranasal dry powder composition is administered if (a) or (b) fails to resume a spontaneous circulation in the patient.

89. The method of claim 88, wherein the intranasal dry powder composition is administered if (a) or (b) fails to resume a spontaneous circulation in the patient within 10 minutes after administration.

90. The method of any one of claims 63-64, wherein the patient is experiencing an allergic reaction.

91. The method of claim 90, wherein the patient is experiencing anaphylaxis.

92. The method of any one of claims 90-91, wherein the intranasal dry powder composition is sufficient to relieve the allergic reaction.

93. The method of claim 92, wherein the intranasal dry powder composition is sufficient to relieve the allergic reaction within in 10 minutes after administration.

94. The method of any one of claims 63-93, wherein the patient is not in a hospital.

95. The method of any one of claims 63-93, wherein the patient is in a hospital.

96. The method of any one of claims 63-93, wherein the patient is in a combat setting.

97. The subject of any claim, that is human.

98. The compositions of claim 1, wherein the anti-anaphylactic or anti-anaphylactoid agent does not comprise cocaine or a derivative thereof.

99. The compositions of claim 9, wherein the vasoactive agent does not comprise cocaine or a derivative thereof.

100. The composition of any one of claims 1-4, in the form of a single dose, which contains about 0.01 mg to about 10 mg of the anti-anaphylactic or anti-anaphylactoid agent.

101. The composition of claim 100, in the form of a single dose, which contains about 0.75 mg, about 1.5 mg, or about 3.0 mg of the anti-anaphylactic or anti-anaphylactoid agent.

102. The composition of any one of claims 9-14, in the form of a single dose, which contains about 0.01 mg to about 10 mg of the vasoactive agent.

103. The composition of claim 102, in the form of a single dose, which contains about 0.75 mg, about 1.5 mg, or about 3.0 mg of the vasoactive agent.

104. The composition of any one of claims 19-22, in the form of a single dose, which contains about 0.01 mg to about 10 mg of the vasodilator.

105. The composition of claim 104, in the form of a single dose, which contains about 0.5 mg or about 1.0 mg of the vasodilator agent.

106. The kit of claim 50, wherein the package comprises one reservoir, wherein the reservoir contains multiple doses of the dry powder composition.

107. The kit of claim 106, wherein the package further comprises one intranasal delivery apparatus.

108. The composition of any one of claims 1-6, wherein the dry powder composition when administered to a patient, reaches a mean AUC_{(0-180 minutes)} of the anti-anaphylactic or anti-anaphylactoid agent which is at least 80% of the mean AUC_{(0-180 minutes)} of a 0.15 mg IV injected anti-anaphylactic or anti-anaphylactoid agent.

109. The composition of any one of claims 1-6, wherein the dry powder composition when administered to a patient, reaches a mean AUC_{(0-inf)} of the anti-anaphylactic or anti-anaphylactoid agent which is at least 80% of the mean AUC_{(0-inf)} of a 0.15 mg IV injected anti-anaphylactic or anti-anaphylactoid agent.

110. The composition of any one of claims 108-109, wherein the IV injected anti-anaphylactic or anti-anaphylactoid agent is epinephrine injected by EpiPen™ autoinjector.

111. The composition of any one of claims 1-6, wherein the dry powder composition when administered to a patient, reaches a mean AUC_{(0-180 minutes)} of the anti-anaphylactic or anti-anaphylactoid agent which is at least 400,000 pg·min/mL.

112. The composition of any one of claims 1-6, wherein the dry powder composition when administered to a patient, reaches a mean AUC_{(0-inf)} of the anti-anaphylactic or anti-anaphylactoid agent which is at least 700,000 pg·min/mL.

113. The composition of any one of claims 9-16, wherein the dry powder composition when administered to a patient, reaches a mean AUC_{(0-180 minutes)} of the vasoactive agent which is at least 80% of the mean AUC_{(0-180 minutes)} of a 0.15 mg IV injected vasoactive agent.

114. The composition of any one of claims 9-16, wherein the dry powder composition when administered to a patient, reaches a mean AUC_{(0-inf)} of the vasoactive agent which is at least 80% of the mean AUC_{(0-inf)} of a 0.15 mg IM injected vasoactive agent.

115. The composition of any one of claims 113-114, wherein the IV injected vasoactive agent is epinephrine injected by EpiPen™ autoinjector.

116. The composition of any one of claims 9-16, wherein the dry powder composition when administered to a patient, reaches a mean AUC_{(0-180 minutes)} of the vasoactive agent which is at least 400,000 pg·min/mL.

117. The composition of any one of claims 9-16, wherein the dry powder composition when administered to a patient, reaches a mean AUC_{(0-inf)} of the vasoactive agent which is at least 700,000 pg·min/mL.

118. A method of dilating a bronchus in a subject, comprising the intranasal administration of the dry powder composition in any one of claims 1-42.

119. The method of claim 118, wherein the dilation occurs without substantial pulmonary inhalation.

120. A method of delivering epinephrine in a subject at least to one of alpha adrenergic receptors, beta adrenergic receptors, or any combination thereof, comprising the intranasal administration of the dry powder composition in any one of claims 1-42.

121. The method of claim 120, wherein the alpha adrenergic receptors consist of the group including alpha-1 and alpha-2 adrenergic receptors.

122. The method of claim 120, wherein the beta adrenergic receptors consist of the group including beta-1, beta-2 and beta-3 adrenergic receptors.

123. The method of claim 120, wherein the delivering is localized.

124. The method of claim 120, wherein the delivering is systematic.

125. The method of claim 63 or 64, wherein the patient is in a civil emergency setting.

126. The method of claim 63 or 64, wherein the patient has a wound.

127. A method of treating a subject with asthma, comprising the intranasal administration of the dry powder composition in any one of claims 1-42.

128. A method of treating a subject with croup, comprising the intranasal administration of the dry powder composition in any one of claims 1-42.

129. A method of treating a condition in a subject by stimulating at least one of alpha adrenergic receptors, beta adrenergic receptors, or any combination thereof, comprising the intranasal administration of the dry powder composition in any one of claims 1-42.

130. A method of treating a subject by increasing the heart rate of the subject, comprising the intranasal administration of the dry powder composition in any one of claims 1-42.

131. A method of treating a subject by increasing the respiratory rate of the subject, comprising the intranasal administration of the dry powder composition in any one of claims 1-42.

132. A method of increasing the blood concentration of epinephrine in a subject, comprising the intranasal administration of the dry powder composition in any one of claims 1-42.

133. A method of treating urticaria in a subject, comprising the intranasal administration of the dry powder composition in any one of claims 1-42.

134. A method of treating pulmonary edema in a subject, comprising the intranasal administration of the dry powder composition in any one of claims 1-42.

135. A method of treating serum sickness in a subject, comprising the intranasal administration of the dry powder composition in any one of claims 1-42.

136. A method of treating a subject with anaphylaxis resulted from an insect bite, comprising the intranasal administration of the dry powder composition in any one of claims 1-42.

137. A method of treating a subject with anaphylaxis resulted from ingested food, comprising the intranasal administration of the dry powder composition in any one of claims 1-42.

138. A method of treating a subject with anaphylaxis resulted from a drug reaction, comprising the intranasal administration of the dry powder composition in any one of claims 1-42.

139. A method of treating itching in a subject, comprising the intranasal administration of the dry powder composition in any one of claims 1-42.

140. A method of counteracting bronchoconstriction effects in a subject following certain chemical exposures, comprising the intranasal administration of the dry powder composition in any one of claims 1-42.

141. The kit of any one of claims 43-62, wherein the dry powder composition has a weight of less than 20 lbs.
